(19) United States
(12) Patent Application Publication
(10) Pub. No.: US 2010/0129303 A1
(43) Pub. Date: May 27, 2010

(54) SUNSCREEN AND PERSONAL CARE COMPOSITIONS COMPRISING A RANDOM TERPOLYMER

(76) Inventors: Olga V. Dueva-Koganov, White Plains, NY (US); Bingham S. Jaynes, New City, NY (US); Joseph A. Lupia, Monroe, NY (US); Arjan T. Termaten, Assen (NL); Marcelles Van der Sluis, Groningen (NL); Marleen Suurmeijer, Joure (NL); David Normington, Leeds (GB); Marcel Schnyder, Birsfelden (CH); Julie Grumelard, Huningue (FR); Myriam Sohn, Saint-Louis (FR)

Correspondence Address: Ciba Corporation;Patent Department, 540 White Plains Road, P.O. Box 2005, Tarrytown, NY, 10591, US

(21) Appl. No.: 12/575,631

(22) Filed: Oct. 8, 2009

Related U.S. Application Data
(60) Provisional application No. 61/196,412, filed on Oct. 17, 2008.

(51) Int. Cl. A61K 8/72; A61Q 17/04

(52) U.S. Cl. 424/60; 424/59

(57) ABSTRACT

Topically applicable, water-resistant cosmetic or dermatological compositions well suited for the UV-photoprotection of human skin and/or hair comprising an effective UV-photoprotecting amount of: (a) at least one UV screening agent from various classes and particularly effect mixtures thereof; and, (b) at least one random terpolymer; and, optionally, (c) other cosmetically acceptable ingredients. The various classes of UV-protecting compounds or UV sunscreens of special interest are selected from the groups b₁ sparingly soluble micronized organic UV absorber, b₂ Bis-Ethylhexyloxyphenol Methoxyphenyl Triazine, b₃ at least one oil soluble organic UV absorber, b₄ at least one inorganic UV absorber and b₅ at least one water soluble UV absorber further defined herein.

The present invention also relates to personal care compositions comprising at least one random terpolymer and other cosmetically acceptable ingredients.

This application claims the benefit of U.S. provisional application No. 61/196,412, filed on Oct. 17, 2008.

### FIELD OF THE INVENTION

The invention is directed to topically applicable, water-resistant cosmetic or dermatological compositions which are well suited for the UV-photoprotection of human skin and/or hair comprising an effective UV-photoprotecting amount of: (a) at least one UV screening agent from various classes and particularly effect mixtures thereof; and, (b) at least one random terpolymer; and, optionally, (c) other cosmetically acceptable ingredients. The various classes of UV-protecting compounds or UV sunscreens of special interest are selected from the groups b₁ sparingly soluble micronized organic UV absorber, b₂ Bis-Ethylhexyloxyphenol Methoxyphenyl Triazine, b₃ at least one oil soluble organic UV absorber, b₄ at least one inorganic UV absorber and b₅ at least one water soluble UV absorber further defined herein.

### BACKGROUND OF THE INVENTION

Sunscreen compositions are applied to the skin to protect the skin from the sun's ultraviolet rays that can lead to erythema, a reddening of the skin also known as sunburn. Sunlight or ultraviolet radiation in the UV-B range has a wavelength of 290 nm to 320 nm and is known to be the primary cause of sunburn. Ultraviolet rays at a wavelength of 320 nm to 400 nm, known as UV-A radiation, produces tanning of the skin. However, in the process of doing so, the UV-A rays can damage or harm the skin.

Besides the immediate malady of sunburn, excessive sunlight exposure can lead to skin disorders. For instance, prolonged and constant exposure to the sun may lead to actinic keratoses and carcinomas. Another long-term effect is premature aging of the skin. This condition is characterized by skin that is wrinkled, cracked and has lost its elasticity.

As stated above, sunscreens are typically formulated with the goal of inhibiting skin damage from the sun's rays. The sunscreen composition filters or blocks the harmful UV-A and UV-B rays that can damage and harm the skin. It is believed that sunscreen agents accomplish this by absorbing the UV-A and/or UV-B rays.

Typically, the above-described oil soluble UV-B filters are combined with the above-described oil soluble UV-A filters in a solution with other lipophilic ingredients and solvents to form an oil phase. Alternative, the above described water soluble UV-B filters are combined with the above-described water-soluble UV-A filters in a solution with other hydrophilic ingredients and solvents to form a water phase. Specific solvents or liquid suncreens are used to solubilize the solid soluble sunscreen actives into the oil or water phase. Particulate UV filters such inorganic pigments or micronized organic UV filters are dispersed in the oil phase or in the water phase or in the finished emulsion depending on their nature and especially on their surface nature.

The final cosmetic compositions are generally manufactured by mixing an oil phase and a water phase, however they can be made without water or oil or composed by other ingredient-based phases (for example: cosmetic oil, clear alcoholic spray, silicone based emulsions). Typically, for oil in water formulation, the oil phase is dispersed into the water phase with the help of emulsifiers and stabilizers, to make an emulsion, which becomes the final sunscreen composition.

A wide variety of cosmetic compositions intended for the photoprotection (UV-A and/or UV-B) of the skin are also known to this art.

Copending U.S. Application Publication Nos. 2008/0247976 and 2008/0247975 herein incorporated entirely by reference, disclose sunscreens in combination with certain copolymers.

U.S. Pat. No. 5,204,090 discloses waterproof sunscreens comprising a water insoluble film forming polymer, which is incorporated herein by reference.

U.S. Pat. No. 5,653,965 discloses film forming polymers for a sunscreen spray, which is incorporated herein by reference.

U.S. Pat. No. 5,487,886 discloses acrylic polymers for sunscreen formulations, which is incorporated herein by reference.

U.S. Pat. No. 5,145,669 discloses water proof sunscreens containing crosslinked copolymer of maleic anhydride, which is incorporated herein by reference.

U.S. Pat. No. 4,663,157 discloses a copolymer of ethylene and acrylic acid for use in sunscreen compositions, which is incorporated herein by reference.

U.S. Publication Application No. 2006/0008427 discloses a photo-protective composition that contains a synergistic combination of a least one sunscreen agent and at least one carotenoid, which is incorporated herein by reference.

U.S. Pat. No. 7,108,860 discloses a cosmetic composition that contains at least two rheology modifying agents, which is incorporated herein by reference.

U.S. Pat. No. 7,014,842 discloses a sunscreen composition comprising one or more photoactive compounds and one or more optimization agents, which is incorporated herein by reference.

U.S. Pat. No. 6,409,998 discloses a UV-photoprotecting emulsion comprising micronized insoluble screening agents and associative polymers, which is incorporated herein by reference.

U.S. Application Publication No. 2004/0126339 discloses a sunscreen composition that includes a mixture of a skin bonding polymer and at least one sunscreen active ingredient, which is incorporated herein by reference.

U.S. Pat. No. 6,312,672 discloses waterproof sunscreen compositions which include polymers of isoprene, butadiene, and/or styrene, which is incorporated herein by reference.

U.S. Application Publication No. 2004/0091434 discloses a topically applicable photostable sunscreen composition containing at least one dibenzoylmethane UV-sunscreen and an effective amount of at least one amphiphilic block copolymer, which is incorporated herein by reference.

U.S. Application Publication No. 2003/0021847 discloses a composition for retaining active ingredients in personal care compositions based on one or more polymers having a network structure in an oil phase, which is incorporated herein by reference.

U.S. Application Publication No. 2002/0076390 discloses a composition for nails, skin and hair in the form of an aqueous emulsion or dispersion, which is incorporated herein by reference.

U.S. Pat. No. 5,688,858 discloses a polymer suitable as a dispersant, which is incorporated herein by reference.

U.S. Application Publication No. 2006/0104923 discloses a sunscreen composition containing fluorinated alkyl ethers, which is incorporated herein by reference.

These anti-sun or sunscreen compositions are quite often provided in the form of an emulsion, of the oil-in-water (0/W) type (namely, a cosmetically and/or dermatologically acceptable carrier comprising an aqueous dispersing continuous phase and a fatty dispersed discontinuous phase) or of the water-in-oil (W/0) type (dispersed aqueous phase in a continuous fatty phase), which contains, at various concentrations, one or more lipophilic conventional organic UV-screening agents and/or inorganic nanopigments of metal oxides, and/or micronized organic UV absorbers, which are suited for selectively absorbing/scattering/reflecting the harmful UV radiation. These screening agents (and the quantities thereof) are selected according to the desired sun protection factor (the sun protection factor (SPF) being mathematically expressed by the ratio of the irradiation time required to attain the erythematogenic threshold with the UV-screening agent to the time required to attain the erythematogenic threshold in the absence of UV-screening agent). In such emulsions, the hydrophilic screening agents are present in the aqueous phase and the lipophilic screening agents are present in the fatty phase.

The oil-in-water emulsions are, in general, more accepted by the consumer than the water-in-oil emulsions because, in particular, of their pleasant feel (similar to water) and their presentation in the form of a non-oily cream or milk; however, they also more readily lose their UV protection efficacy as soon as they come into contact with water. Indeed, the hydrophilic screening agents tend to disappear in water, upon washing in the sea or in a swimming pool, under the shower or when engaged in water sports; thus, anti-sun or sunscreen compositions containing same, whether alone or combined with lipophilic screening agents, no longer provide the desired initial protection as soon as the substrate (skin or hair) to which they have been applied is contacted with water.

Anti-sun (sunscreen) compositions exhibiting improved resistance to water are formulated as water-in-oil, oil-in-water emulsions and alcoholic sprays. Indeed, a hydrophilic screening agent is more stable to water in a water-in-oil emulsion than in an oil-in-water emulsion. However, as indicated above, such compositions are not yet completely satisfactory since they promote, after application, a fat-like impression which is particularly unpleasant for the user.

Thus, serious need continues to exist for anti-sun or sunscreen compositions which impart to the skin and/or the hair effective solar protection which is stable over time and resistant to water (stability to water) and the cosmetic performance of which presents features that would be comparable to those obtained with conventional oil/water emulsions.

### SUMMARY OF THE INVENTION

It is now surprisingly and unexpectedly determined that specific sunscreen compositions containing at least one UV-screening agent and at least one random terpolymer not only provide anti-sun compositions whose cosmetic performance features are comparable to those generally associated with a conventional sunscreen composition formulated as an oil/water emulsion, but also exhibit good stability, water-resistance as well as enhanced stability to water.

Therefore, a first aspect of the present invention is directed to a sunscreen composition comprising at least one sunscreen agent, at least one random terpolymer of formula (I), and other cosmetically acceptable ingredients.

An important aspect of the present invention is the discovery that certain combinations of UV absorbers with formula (I) or select random terpolymers (Ia) give better performance in regard to water-resistance of films formed on skin or hair as well as improved sunscreening efficacy.

Therefore the invention is directed to certain select terpolymers and UV absorbers selected from specific classes of UV absorbers. Thus the invention embodies:

A) at least one random terpolymer of formula (Ia)

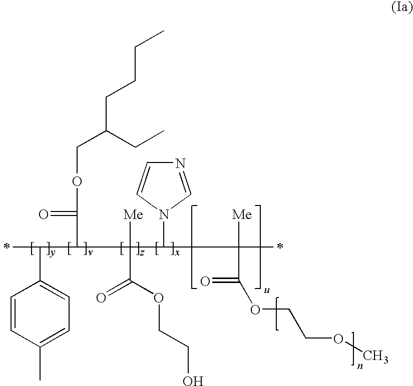

wherein
y, v, u, z and x represent the percentage by weight that each repeating unit or derived monomer is contained within the terpolymer;
y, v, u, z and x preferably add up to total 100 weight percent relative to the total weight of the terpolymer;
y is from about 1 to about 30%, preferably about 5 to about 20 and most preferably about 6 to about 10% by weight of the terpolymer;
v is from about 5% to about 75% by weight of the terpolymer, preferably about 5 to about 50, most preferably about 8 to about 20% by weight of the terpolymer;
u is from about 20% to about 80%, preferably about 30% to about 75% and most preferably about 40 to about 75% by weight of the terpolymer;
z is from about 1% to about 40%, preferably about 2% to about 15% and most preferably about 3% to about 10% by weight of the terpolymer;
x is from about 5% to about 25%, preferably about 6% to about 20%, most preferably about 8% to about 15% by weight of the terpolymer;
* is a terminal group, for example, a catalyst residue;
n is 2 to 20, preferably, 4 to 18 and most preferably 6 to 12;
wherein formula (I) is characterized by a weight average molecular weight of from about 5,000 to about 50,000, preferably about 10,000 to about 40,000 and most preferably about 15,000 to about 30,000 Daltons
and
B) a sunscreen selected from group consisting of
b₁ at least one sparingly soluble micronized organic UV absorber,
b₂ Bis-Ethylhexyloxyphenol Methoxyphenyl Triazine,
b₃ at least one oil soluble organic UV absorber,
b₄ at least one inorganic UV absorber,
b₅ at least one water soluble UV absorber,
and
mixtures thereof.

The sunscreens are defined selectively as:

b₁) the at least one sparingly soluble micronized UV absorber is selected from the group consisting of:
Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- and mixtures thereof. The b₁ UV absorbers are sparingly soluble and are micronized or unmicronized, but preferably micronized.

Sparingly soluble in regard to b₁ means that the UV absorber is not appreciably soluble in either water or oil.

b₂) is Bis-Ethylhexyloxyphenol Methoxyphenyl Triazine;
b₃) the at least one oil soluble organic UV absorber is selected from the group consisting of:
Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, Diethylamino Hydroxy Benzoyl Hexyl Benzoate (DHHB) and mixtures thereof;

Soluble in regard to b₃ means that the UV absorber is at least partially soluble in oil or organic solvents.

b₄) the at least one inorganic UV absorber is selected from the group consisting of: titanium oxide, zinc oxide and mixtures thereof;
and
b₅) the at least one water soluble UV absorber is Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, PEG-25 PABA and mixtures thereof.

Select combinations of UV absorbers with formula (I), defined below or the select terpolymer of formula (Ia) defined above are also embodied by the present invention.

Especially important are select UV absorber combinations with the select terpolymer as these combinations have been discovered to give heightened SPF values and high water-resistance when applied to skin and hair.

For example improved sunscreening effects are observed

when component B comprises both b₁ and b₂ with the select terpolymer;
when component B) comprises (b₃) titanium dioxide or zinc oxide with the proviso that the sunscreen composition contains substantially no organic UV absorbers.

Further select of combination are for example combinations of component B with the formula (I), defined below or select terpolymer of formula (Ia) above and comprise the UV absorber combination selected from the group of UV absorber combinations consisting of

Methylene Bis-Benzotriazolyl Tetramethylbutylphenol/Tris-Biphenyl Triazine/Methanone, 1,1′-(1,4-piperazinediyl)bis[1-[2-[4-(diethylamino)-2-hydroxybenzoyl]phenyl].
and
Methylene Bis-Benzotriazolyl Tetramethylbutylphenol/Tris-Biphenyl Triazine/Methanone, 1,1′-(1,4-piperazinediyl)bis[1-[2-[4-(diethylamino)-2-hydroxybenzoyl]phenyl]-/Titanium oxide,
with the proviso that the sunscreen composition contains substantially no soluble organic UV absorber
and
Methylene Bis-Benzotriazolyl Tetramethylbutylphenol, Tris-Biphenyl Triazine and Methanone, 1,1′-(1,4-piperazinediyl)bis[1-[2-[4-(diethylamino)-2-hydroxybenzoyl]phenyl]- are sparingly soluble micronized UV absorbers.

The proviso above stating “no soluble organic UV absorbers” means no oil or water soluble organic UV absorber are part of the sunscreen composition.

A further select combination of component B) with the terpolymer of formula (I), defined below or the select terpolymer of formula (Ia) may comprise a UV absorber combination comprising

i.) 0.1 to 20 wt. % based on the total weight of the sunscreen composition of a UV absorber selected from group consisting of
Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- and Bis-Ethylhexyloxyphenol Methoxyphenyl Triazine;
and
ii.) 0.1 to 20 wt. % based on the total weight of the sunscreen composition of a UV absorber selected from group consisting of
Diethylamino Hydroxy Benzoyl Hexyl Benzoate (DHHB), Diethylhexyl Butamido Triazone (DBT), Ethylhexyl Methoxycinnamate (EHMC), Ethylhexyl Salicylate (EHS), Ethylhexyl Triazone (EHT), Octocrylene (OCR), Titanium Dioxide and Phenylbenzimidazole Sulfonic Acid (PBSA).
and Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- are sparingly soluble micronized UV absorbers.

A further select combination of component B) with the terpolymer of formula (I) or the select formula (Ia) may comprise a UV absorber combination comprising

i.) 0.1 to 20 wt. % based on the total weight of the sunscreen composition of a UV absorber selected from group consisting of Methylene Bis-Benzotriazolyl Tetramethylbutylphenol, Tris-Biphenyl Triazine, Methanone, piperazinediyl)bis[1-[2-[4-(diethylamino)-2-hydroxybenzoyl]phenyl]- and Bis-Ethylhexyloxyphenol Methoxyphenyl Triazine;
wherein Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- are sparingly soluble micronized UV absorbers;
ii.) 0.1 to 20 wt. % based on the total weight of the sunscreen composition of the UV absorber Butyl Methoxydibenzoylmethane (BMBM);
iii.) 0.1 to 20 wt. % based on the total weight of the sunscreen composition of the UV absorber selected from group consisting of

### Ethylhexyl Triazone (EHT) and Octocrylene (OCR);

and
iv) 0 to 20 wt. % based on the total weight of the sunscreen composition of
Ethylhexyl Methoxycinnamate (EHMC),
Ethylhexyl Salicylate (EHS),
Diethylhexyl Butamido Triazone (DBT),
Phenylbenzimidazole Sulfonic Acid (PBSA)
or
Titanium Dioxide.

Specific combinations of UV absorber mixtures selected from groups b₁, b₂, b₃, b₄ and b₅ in combination with terpolymer of formula (I) or the selective formula (Ia) are for example:
Bis-Ethylhexyloxyphenol Methoxyphenyl Triazine/Methylene Bis-Benzotriazolyl Tetramethylbutylphenol/Butyl Methoxydibenzoylmethane (BMBM);
Bis-Ethylhexyloxyphenol Methoxyphenyl Triazine/Butyl Methoxydibenzoylmethane (BMBM);
Methylene Bis-Benzotriazolyl Tetramethylbutylphenol/Butyl Methoxydibenzoylmethane (BMBM);
Bis-Ethylhexyloxyphenol Methoxyphenyl Triazine/Methylene Bis-Benzotriazolyl Tetramethylbutylphenol/Ethylhexyl Methoxycinnamate (EHMC);
Bis-Ethylhexyloxyphenol Methoxyphenyl Triazine/Ethylhexyl Methoxycinnamate (EHMC);
Methylene Bis-Benzotriazolyl Tetramethylbutylphenol/Ethylhexyl Methoxycinnamate (EHMC);
Bis-Ethylhexyloxyphenol Methoxyphenyl Triazine/Methylene Bis-Benzotriazolyl Tetramethylbutylphenol/Octocrylene (OCR);
Bis-Ethylhexyloxyphenol Methoxyphenyl Triazine/Octocrylene (OCR);
Methylene Bis-Benzotriazolyl Tetramethylbutylphenol/Octocrylene (OCR);
Bis-Ethylhexyloxyphenol Methoxyphenyl Triazine/Methylene Bis-Benzotriazolyl Tetramethylbutylphenol/Phenylbenzimidazole Sulfonic Acid (PBSA);
Bis-Ethylhexyloxyphenol Methoxyphenyl Triazine/Phenylbenzimidazole Sulfonic Acid (PBSA);
Methylene Bis-Benzotriazolyl Tetramethylbutylphenol/Phenylbenzimidazole Sulfonic Acid (PBSA);
Bis-Ethylhexyloxyphenol Methoxyphenyl Triazine/Methylene Bis-Benzotriazolyl Tetramethylbutylphenol/Ethylhexyl Salicylate (EHS);
Bis-Ethylhexyloxyphenol Methoxyphenyl Triazine/Ethylhexyl Salicylate (EHS);
Methylene Bis-Benzotriazolyl Tetramethylbutylphenol/Ethylhexyl Salicylate (EHS);
Bis-Ethylhexyloxyphenol Methoxyphenyl Triazine/Methylene Bis-Benzotriazolyl Tetramethylbutylphenol/Diethylhexyl Butamido Triazone (DBT);
Bis-Ethylhexyloxyphenol Methoxyphenyl Triazine/Diethylhexyl Butamido Triazone (DBT);
Methylene Bis-Benzotriazolyl Tetramethylbutylphenol/Diethylhexyl Butamido Triazone (DBT);
Bis-Ethylhexyloxyphenol Methoxyphenyl Triazine/Methylene Bis-Benzotriazolyl Tetramethylbutylphenol/Titanium Dioxide;
Bis-Ethylhexyloxyphenol Methoxyphenyl Triazine/Titanium Dioxide;
   wherein, Benzotriazolyl Tetramethylbutylphenol is an sparingly soluble micronized UV absorber.

Alternative specific examples of b₁, b₂, b₃, b₄ and b₅ combinations with formula (I) or the selective random terpolymer (Ia) are:
Methylene Bis-Benzotriazolyl Tetramethylbutylphenol/Bis-Ethylhexyloxyphenol
Methoxyphenyl Triazine/Butyl Methoxydibenzoylmethane (BMBM)/Octocrylene;
Methylene Bis-Benzotriazolyl Tetramethylbutylphenol/Butyl Methoxydibenzoylmethane (BMBM)/Octocrylene;
Bis-Ethylhexyloxyphenol Methoxyphenyl Triazine/Butyl Methoxydibenzoylmethane (BMBM)/Octocrylene;
Methylene Bis-Benzotriazolyl Tetramethylbutylphenol/Bis-Ethylhexyloxyphenol Methoxyphenyl Triazine/Butyl Methoxydibenzoylmethane (BMBM)/Ethylhexyl Triazone (EHT);
Methylene Bis-Benzotriazolyl Tetramethylbutylphenol/Butyl Methoxydibenzoylmethane (BMBM)/Ethylhexyl Triazone (EHT);
Bis-Ethylhexyloxyphenol Methoxyphenyl Triazine/Butyl Methoxydibenzoylmethane (BMBM)/Ethylhexyl Triazone (EHT);
Methylene Bis-Benzotriazolyl Tetramethylbutylphenol/Bis-Ethylhexyloxyphenol Methoxyphenyl Triazine/Butyl Methoxydibenzoylmethane (BMBM)/Octocrylene/Ethylhexyl Methoxycinnamate (EHMC);
Methylene Bis-Benzotriazolyl Tetramethylbutylphenol/Butyl Methoxydibenzoylmethane (BMBM)/Octocrylene/Ethylhexyl Methoxycinnamate (EHMC);
Bis-Ethylhexyloxyphenol Methoxyphenyl Triazine/Butyl Methoxydibenzoylmethane (BMBM)/Octocrylene/Ethylhexyl Methoxycinnamate (EHMC);
Methylene Bis-Benzotriazolyl Tetramethylbutylphenol/Bis-Ethylhexyloxyphenol Methoxyphenyl Triazine/Butyl Methoxydibenzoylmethane (BMBM)/Ethylhexyl Triazone (EHT)/Ethylhexyl Methoxycinnamate (EHMC);
Methylene Bis-Benzotriazolyl Tetramethylbutylphenol/Butyl Methoxydibenzoylmethane (BMBM)/Ethylhexyl Triazone (EHT)/Ethylhexyl Methoxycinnamate (EHMC);
Bis-Ethylhexyloxyphenol Methoxyphenyl Triazine/Butyl Methoxydibenzoylmethane (BMBM)/Ethylhexyl Triazone (EHT)/Ethylhexyl Methoxycinnamate (EHMC);
Methylene Bis-Benzotriazolyl Tetramethylbutylphenol/Bis-Ethylhexyloxyphenol Methoxyphenyl Triazine/Butyl Methoxydibenzoylmethane (BMBM)/Octocrylene/Ethylhexyl Salicylate (EHS);
Methylene Bis-Benzotriazolyl Tetramethylbutylphenol/Butyl Methoxydibenzoylmethane (BMBM)/Octocrylene/Ethylhexyl Salicylate (EHS);
Bis-Ethylhexyloxyphenol Methoxyphenyl Triazine/Butyl Methoxydibenzoylmethane (BMBM)/Octocrylene/Ethylhexyl Salicylate (EHS);
Methylene Bis-Benzotriazolyl Tetramethylbutylphenol/Bis-Ethylhexyloxyphenol Methoxyphenyl Triazine/Butyl Methoxydibenzoylmethane (BMBM)/Ethylhexyl Triazone (EHT)/Ethylhexyl Salicylate (EHS);
Methylene Bis-Benzotriazolyl Tetramethylbutylphenol/Butyl Methoxydibenzoylmethane (BMBM)/Ethylhexyl Triazone (EHT)/Ethylhexyl Salicylate (EHS);
Bis-Ethylhexyloxyphenol Methoxyphenyl Triazine/Butyl Methoxydibenzoylmethane (BMBM)/Ethylhexyl Triazone (EHT)/Ethylhexyl Salicylate (EHS);
   wherein Methylene Bis-Benzotriazolyl Tetramethylbutylphenol is an sparingly soluble micronized UV absorber.

Specific combinations of UV absorber mixtures selected from groups b₁, b₂, b₃, b₄ and b₅ in combination with formula (I) or selective formula (Ia) are for example:
Methylene Bis-Benzotriazolyl Tetramethylbutylphenol/Bis-Ethylhexyloxyphenol Methoxyphenyl Triazine/Diethylamino Hydroxy Benzoyl Hexyl Benzoate (DHHB);
Methylene Bis-Benzotriazolyl Tetramethylbutylphenol/Diethylamino Hydroxy Benzoyl Hexyl Benzoate (DHHB);
Bis-Ethylhexyloxyphenol Methoxyphenyl Triazine/Diethylamino Hydroxy Benzoyl Hexyl Benzoate (DHHB);
Methylene Bis-Benzotriazolyl Tetramethylbutylphenol/Diethylamino Hydroxy Benzoyl Hexyl Benzoate (DHHB)/Ethylhexyl Methoxycinnamate (EHMC);
Bis-Ethylhexyloxyphenol Methoxyphenyl Triazine/Diethylamino Hydroxy Benzoyl Hexyl Benzoate (DHHB)/Ethylhexyl Methoxycinnamate (EHMC);
Methylene Bis-Benzotriazolyl Tetramethylbutylphenol/Bis-Ethylhexyloxyphenol Methoxyphenyl Triazine/Diethylamino Hydroxy Benzoyl Hexyl Benzoate (DHHB)/Ethylhexyl Methoxycinnamate (EHMC);
   wherein, Methylene Bis-Benzotriazolyl Tetramethylbutylphenol is an sparingly soluble micronized UV absorber.

A particular surprising discovery by the inventors also embodies substantially anhydrous sunscreen alcoholic formulations, which incorporates the terpolymer of formula (I) or selective random terpolymer (Ia) in combination with UV absorbers and combinations of UV absorbers.

The substantially anhydrous sunscreen alcoholic formulations work at very low concentrations of the random terpolymer of formula (I) and the select formula (Ia). For example, ranges from about 0.0001 to about 0.0500 weight percent of the substantially anyhydrous sunscreen alcoholic formulation show significant improvements in increasing SPF ratings on skin and hair.

The substantially anhydrous sunscreen alcoholic formulation which incorporates the terpolymer of formula (I) or selective terpolymer of formula (Ia) is especially efficient when the UV absorber is selected from at least one oil soluble organic UV absorber (b₃).

A second aspect of the present invention is directed to a method of preparing a sunscreen composition comprising mixing together at least one sunscreen agent, at least one random terpolymer of formula (Ia) as described above and, optionally, other cosmetically acceptable ingredients.

A third aspect of the present invention is directed to a method of increasing the sun protection factor of a sunscreen composition wherein said method comprises incorporating into said compositions above at an effective amount of at least one select random terpolymer according to formula (Ia) described above.

In particular a method of improving sunscreen efficacy on hair and skin is envisioned.

Thus the invention is directed to a method of improving sunscreen efficacy on hair and skin which method comprises

applying the compositions described above onto said hair or skin.

Further a method of improving the water-resistance of a sunscreen composition on skin or hair is envisioned.

Thus the invention is directed to a method of improving the water-resistance of a sunscreen composition on skin or hair which method comprises

applying to said skin or hair the compositions described above.

A fourth aspect of the present invention is directed to a method of improved UV protection of mammalian hair and/or skin from the damaging effects of UV radiation wherein said method comprises applying to said skin and/or said hair an effective amount of a sunscreen composition comprising at least one sunscreen agent or the combinations of sunscreen agents described above, at least the select random terpolymer of formula (Ia) and, optionally, other cosmetically acceptable ingredients.

A fifth aspect of the present invention is directed to a cosmetic or dermatological composition comprising a random terpolymer of formula (Ia) and other cosmetically acceptable ingredients.

### DETAILED DESCRIPTION OF THE INVENTION

### Definitions

Formula (Ia) refers to the select terpolymer described above under “Summary of the Invention”.

Formula (I) without the “a” refers to the more generic formula (I) defined below.

In describing the UV absorbers and combinations of UV absorbers used with either Formula (I) or the selective Formula (Ia), the inventors have chosen to group the UV absorbers into various categories.

These categories are:
b₁ at least one sparingly soluble micronized organic UV absorber,
b₂ Bis-Ethylhexyloxyphenol Methoxyphenyl Triazine,
b₃ at least one oil soluble organic UV absorber,
b₄ at least one inorganic UV absorber,
b₅ at least one water soluble UV absorber.

Sparingly soluble in regard to ID, means that the UV absorber is not appreciably soluble in either water or oil. However, b₁ may be dispersible in oil or water and therefore be described as water-dispersible, oil-dispersible or both oil and water-dispersible.

Oil soluble organic UV absorber in regard to b₃ means that b₃ has appreciable solubility in oil or organic solvents.

Oils for purposes of the invention means an oil or wax or mixtures thereof. These oils or waxes may comprise fatty acids, fatty alcohols and esters of fatty acids. The oils may be selected from among animal, vegetable, mineral or synthetic oils and, in particular, from among liquid paraffin, paraffin oil, silicone oils, volatile or otherwise, isoparaffins, polyolefins, fluorinated or perfluorinated oils. Likewise, the waxes may be animal, fossil, vegetable, mineral or synthetic waxes which are also known per se.

Organic solvents for purposes of the invention means solvents which are acceptable for use in cosmetics or personal care products and are well known to those familiar with the art. Exemplary organic solvents include the lower alcohols and polyols.

Water soluble in regard to b₅ is understood to mean the UV absorber has appreciable solubility in water.

“Monomer” means an ethylenically unsaturated compound before polymerization.

“Monomer unit” means the unit formed by the ethylenically unsaturated compound after polymerization.

Substantially anhydrous in regard to alcoholic sunscreens for purposes of the invention means no water is present or very low levels of water are present in the alcoholic sunscreen formulation. For example, no more than 0.1 wt. %, or 0.5 wt. % or 1 wt. % of water is present in the alcoholic sunscreen formulation.

### Generic Formula (I)

The present invention provides a sunscreen composition comprising:

(A) at least one random terpolymer of formula (I)

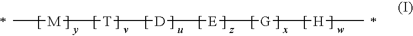

(B) at least one UV screening agent;
wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
x is from about 1% to about 50% by weight of the terpolymer;
w is from about 0% to about 50% by weight of the terpolymer;
* is a terminal group, for example, a catalyst residue;
M, T, D, E, G, and H are covalently bonded to each other;
M is derived from at least one monomer of formula (II)

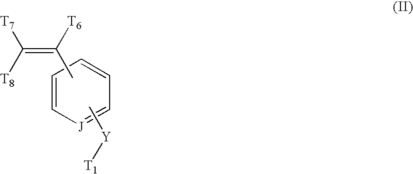

wherein T6, T7, and T8 are C1-C4 alkyl or hydrogen; Y is a direct bond, —O—, —S—, —N(H)—or —N(T1)-; T1 is hydrogen or C1-C4 alkyl; and J is a nitrogen or carbon atom;
T, D, and E are independently derived from at least one monomer of formula (III)

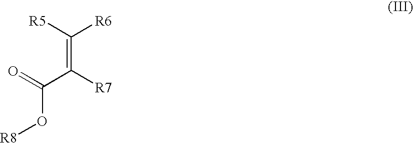

wherein R5, R6 and R7 may be the same or different and represent hydrogen or C1-C22 alkyl;
R8 is C1-C30 alkyl, C6-C15 cycloalkyl, or C6-C15 aryl; said substituted alkyl, said cycloalkyl or said aryl may also be substituted by one or more OH and/or NH2 groups; or said alkyl or said cycloalkyl may be interrupted by one or more —O— groups and/or —N(H)— groups;
G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, and acrylonitrile;
and
(c) other cosmetically acceptable ingredients,
with the proviso that T, D, and E are different from each other.

### Select Formula (Ia)

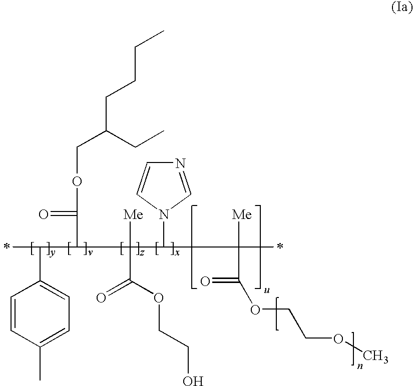

wherein
y, v, u, z and x represent the percentage by weight that each repeating unit or derived monomer is contained within the terpolymer;
y, v, u, z and x preferably add up to total 100 weight percent relative to the total weight of the terpolymer;
y is from about 1 to about 30%, preferably about 5 to about 20 and most preferably about 6 to about 10% by weight of the terpolymer;
v is from about 5% to about 75% by weight of the terpolymer, preferably about 5 to about 50, most preferably about 8 to about 20% by weight of the terpolymer;
u is from about 20% to about 80%, preferably about 30% to about 75% and most preferably about 40 to about 75% by weight of the terpolymer;
z is from about 1% to about 40%, preferably about 2% to about 15% and most preferably about 3% to about 10% by weight of the terpolymer;
x is from about 5% to about 25%, preferably about 6% to about 20%, most preferably about 8% to about 15% by weight of the terpolymer;
* is a terminal group, for example, a catalyst residue;
n is 2 to 20, preferably, 4 to 18 and most preferably 6 to 12;
wherein formula (I) is characterized by a weight average molecular weight of from about 5,000 to about 50,000, preferably about 10,000 to about 40,000 and most preferably about 15,000 to about 30,000 Daltons
and
B) a sunscreen selected from group consisting of
b₁ at least one sparingly soluble micronized organic UV absorber,
b₂ Bis-Ethylhexyloxyphenol Methoxyphenyl Triazine,
b₃ at least one oil soluble organic UV absorber,
b₄ at least one inorganic UV absorber,
b₅ at least one water soluble UV absorber,
and
mixtures thereof.

The above select formula (Ia) may include additional monomers. The monomer units making up the terpolymer of formula (Ia) may be formed after initial polymerization. For example, the monomer units “u” may be formed from transesterification of a methyl ester with a polyethylene monoglycol.

* represents a catalyst residue for example, but may also be virtually any endcapping group which terminates the polymer chain. These endcapping groups may for example be an —O-alkyl or O—C(O)-alkyl endcapping group. The alkyl for example may be branched or unbranched and range from C₁-C₂₀.

### UV Absorbers

In various embodiments the present invention may require for example at least one, two, three or more UV absorbers.

For example component (B) may comprise a single type of UV absorber or any combination of UV absorbers grouped below combined with the generic formula (I) or the more selective formula (Ia):
b₁ is at least one sparingly soluble micronized organic UV absorber,
b₂ Bis-Ethylhexyloxyphenol Methoxyphenyl Triazine,
b₃ at least one oil soluble organic UV absorber,
b₄ at least one inorganic UV absorber,
b₅ at least one water soluble UV absorber,
and
mixtures thereof.
   UV Absorber b₁

A preferred class of micronized UV filters (B) according to component (b₁) are triazine derivatives of formula

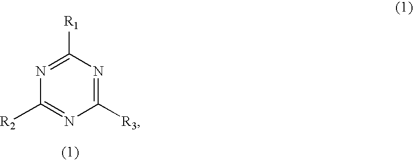

wherein
R₁, R₂ and R₃ independently from each other are a radical of formula

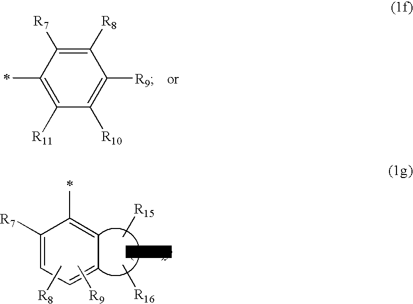

R₇ and R₁₁ independently from each other are hydrogen; C₁-C₁₅alkyl; or C₆-C₁₂aryl;
R₈, R₉ and R₁₀, independently from each other, are hydrogen; or a radical of formula

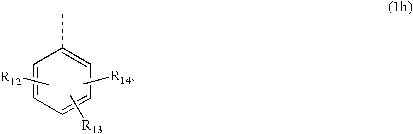

wherein, in formula (1f), at least one of the radicals R₈, R₉ and R₁₀ are a radical of formula (1h);
R₁₂, R₁₃, R₁₄, R₁₅ and R₁₆ independently from each other are hydrogen; hydroxy; halogen; C₁-C₁₈alkyl; C₁-C₁₈alkoxy; C₆-C₁₂aryl; biphenylyl; C₆-C₁₂aryloxy; C₁-C₁₈alkylthio; carboxy; —COOM; C₁-C₁₈-alkylcarboxyl; aminocarbonyl; or mono- or di-C₁-C₁₈alkylamino; C₁-C₁₀acylamino; —COON;
M is an alkali metal ion;
x is 1 or 2; and
y is a number from 2 to 10.

Most preferred triazine derivatives are compounds of formula

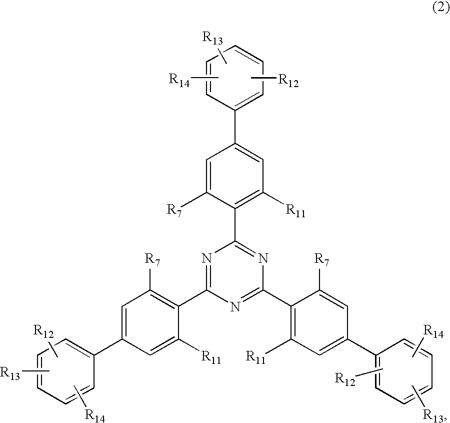

wherein
R₇, R₁₁, R₁₂, R₁₃ and R₁₄ are defined as in formula (1f), (1g) or (1h), and most preferably compound of formula (2), wherein
R_{7 and} R₁₁ are hydrogen.

Furthermore, triazine derivatives of formula

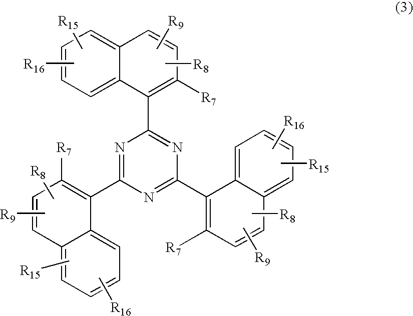

are preferred, wherein
R₇, R₈, R₉, R₁₅ and R₁₆ are defined as in formula (1g), and most preferably compounds of formula (3), wherein
R₇, R₈, R₉, R₁₅ and R₁₆ are hydrogen; or, independently from each other, C₁-C₁₈alkyl.

Most preferred as component (a) are triazine derivatives of formula

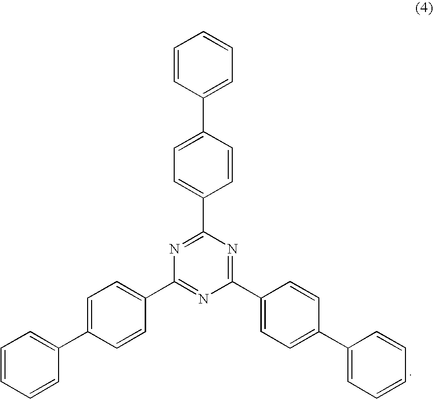

One preferred class of benzotriazole micronized organic UV absorbers is that having the formula

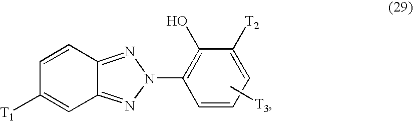

wherein
T₁ is C₁-C₃alkyl or, preferably, hydrogen; or a radical of formula

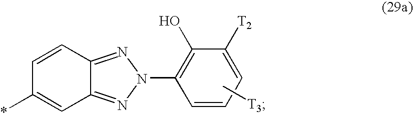

and
T₂ and T₃, independently from each other are C₁-C₁₂alkyl, preferably i-octyl; or C₁-C₄alkyl substituted by phenyl, preferably α,α-dimethylbenzyl.

A still further preferred class of benzotriazole micronized organic UV absorbers corresponds to the formula

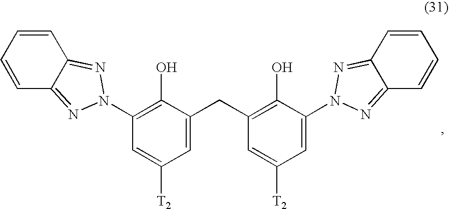

wherein
T₂ is hydrogen; C₁-C₁₂alkyl, preferably iso-octyl, or C₁-C₄alkyl substituted by phenyl, preferably α,α-dimethylbenzyl.

A preferred class of benzophenones is the amino substituted hydroxylphenyl benzophenone derivatives taught in PCT publication No. WO04052837. Such amino substituted hydroxylphenyl benzophenones are

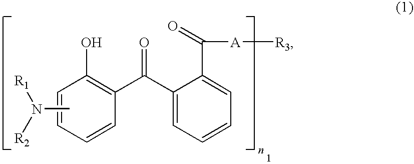

wherein
R₁ and R₂ independently from each other are; C₁-C₂₀alkyl; C₂-C₂₀alkenyl; C₃-C₁₀cycloalkyl; C₃-C₁₀cycloalkenyl; or R₁ and R₂ together with the linking nitrogen atom form a 5- or 6-membered heterocyclic ring;
n₁ is a number from 1 to 4;
   when n₁=1,
R₃ is a saturated or unsaturated heterocyclic radical; hydroxy-C₁-C₅alkyl; cyclohexyl optionally substituted with one or more C₁-C₅alkyl; phenyl optionally substituted with a heterocyclic radical, aminocarbonyl or C₁-C₅alkylcarboxy;
   when n₁ is 2,
   R₃ is an alkylene-, cycloalkylene, alkenylene or phenylene radical which is optionally substituted by a carbonyl- or carboxy group; a radical of formula .—CH₂—C≡C—CH₂—. or R₃ together with A forms a bivalent radical of the formula

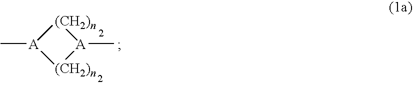

wherein
n₂ is a number from 1 to 3;
   when n₁ is 3,
R₃ is an alkantriyl radical;
   when n₁ is 4,
R₃ is an alkantetrayl radical;
A is —O—; or N(R₅)—; and
R₅ is hydrogen; C₁-C₅alkyl; or hydroxy-C₁-C₅alkyl.

C₁-C₂₀Alkyl denotes a linear or branched, unsubstituted or substituted alkyl group such as, for example, methyl, ethyl, propyl, isopropyl, n-butyl, n-hexyl, cyclohexyl, n-decyl, n-dodecyl, n-octadecyl, eicosyl, methoxyethyl, ethoxypropyl, 2-ethylhexyl, hydroxyethyl, chloropropyl, N,N-diethylaminopropyl, cyanoethyl, phenethyl, benzyl, p-tert-butylphenethyl, p-tert-octylphenoxyethyl, 3-(2,4-di-tert-amylphenoxy)-propyl, ethoxycarbonylmethyl-2-(2-hydroxyethoxy)ethyl or 2-furylethyl.

C₂-C₂₀alkenyl is for example allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, iso-dodecenyl, n-dodec-2-enyl or n-octadec-4-enyl.

C₃-C₁₀cycloalkyl is for example cyclopropyl, cyclobutyl, cyclopentyl, cycloheptyl, cyclooctyl, cyclononyl or cyclodecyl and preferably cyclohexyl. These radicals may besubstituted, for example by one or more oder equal or different C₁-C₄alkyl radicals, preferably by methyl, and/or hydroxy. If cycloalkyl radicals are substituted by one or more radicals, they are preferably substituted by one, two or four, preferably by one or two equal or radicals.

C₃-C₁₀cycloalkenyl is for example cyclopropenyl, cyclobutenyl, cyclopentenyl, cycloheptenyl, cycloocentyl, cyclononenyl or cyclodecenyl and preferably cyclohexenyl. These radicals may be substituted with one or more equal or different C₁-C₄alkyl radical, preferably with methyl, and/or hydroxy. If cycloalkenyl radicals are substituted with one or more radicals they are preferably substituted with one, two, three or four, preferably with one or two equal or different radicals.

Hydroxy substituted C₁-C₅alkyl groups are for example hydroxymethyl, hydroxyethyl, hydroxypropyl, hydroxybutyl or hydroxypentyl.

An alkylene radical is preferably a C₁-C₁₂alkylene radical, like for example methylene, ethylene, propylene, butylene, hexylene or octylene.

The alkylene radicals may optionally be substituted by one or more C₁-C₅alkyl radicals.

If R₁ and R₂ are heterocyclic radicals, these comprise one, two, three or four equal or different ring hetero atoms. Special preference is given to heterocycles which contain one, two or three, especially one or two, identical or different hetero atoms. The heterocycles may be mono- or poly-cyclic, for example mono-, bi- or tri-cyclic. They are preferably mono- or bi-cyclic, especially monocyclic. The rings preferably contain 5, 6 or 7 ring members. Examples of monocyclic and bicyclic heterocyclic systems from which radicals occurring in the compounds of formula (1) or (2) may be derived are, for example, pyrrole, furan, thiophene, imidazole, pyrazole, 1,2,3-triazole, 1,2,4-triazole, pyridine, pyridazine, pyrimidine, pyrazine, pyran, thiopyran, 1,4-dioxane, 1,2-oxazine, 1,3-oxazine, 1,4-oxazine, indole, benzothiophene, benzofuran, pyrrolidine, piperidine, piperazine, morpholine and thiomorpholine.

The preferred list of sparingly soluble micronized organic UV absorbers which may be combined

with the terpolymer of formula (I) or select terpolymer (Ia) are defined under the category b1 and are:
Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- and mixtures thereof.

Sparingly soluble organic compounds which are used in the present invention are present in the micronized state. They may be prepared by any known process suitable for the preparation of microparticles, for example:
wet-milling (low-viscosity micronisation process for pumpable dispersions), with a hard grinding medium, for example zirconium silicate balls in a ball mill, and a protective surfactant or a protective polymer in water or in a suitable organic solvent;
wet-kneading (high-viscosity micronisation process for non-pumpable pastes) using a continuous or discontinuous (batch) kneader. For a wet-kneading process, a solvent (water or cosmetically acceptable oils), a grinding aid (surfactant, emulsifier) and a polymeric grinding aid may be used.
spray-drying from a suitable solvent, for example aqueous suspensions or suspensions containing organic solvents, or true solutions in water, ethanol, dichloroethane, toluene or N-methylpyrrolidone etc.
by expansion according to the RESS process (Rapid Expansion of Supercritical Solutions) of supercritical fluids (e.g. CO₂) in which the UV filter or filters is/are dissolved, or the expansion of liquid carbon dioxide together with a solution of one or more UV filters in a suitable organic solvent;
by reprecipitation from suitable solvents, including supercritical fluids (GASR process=Gas Anti-Solvent Recrystallisation/PCA process=Precipitation with Compressed Anti-solvents).

As milling apparatus for the preparation of the sparingly soluble micronised organic compounds there may be used, for example, a jet mill, ball mill, vibratory mill or hammer mill, preferably a high-speed mixing mill. Even more preferable mills are modern ball mills; manufacturers of these types of mill are, for example, Netzsch (LMZ mill), Drais (DCP-Viscoflow or Cosmo), Bühler AG (centrifugal mills) or Bachhofer.

Examples of kneading apparatus for the preparation of the micronised organic UV absorbers are typical sigma-blade batch kneaders but also serial batch kneaders (IKA-Werke) or continuous kneaders (Continua from Werner and Pfleiderer).

The grinding of the sparingly soluble organic compounds used in the present invention is preferably carried out with a grinding aid.

The dispersing agent (b) may be used as a low molecular weight grinding aid for all the above micronisation processes.

Useful anionic, non-ionic or amphoteric surfactants are disclosed below in the sections entitled “specific dispersing agents”.

Preferred useful grinding aids for an aqueous dispersion are anionic surfactants with a HLB (Hydrophile-Lipophile Balance) value higher than 8, more preferably higher than 10.

Any conventionally usable anionic, non-ionic or amphoteric surfactants (component (b)) can be used as dispersing agents. Such surfactant systems may comprise for example: carboxylic acids and their salts: alkaline soap of sodium, potassium and ammonium, metallic soap of calcium or magnesium, organic basis soap such as Lauric, myristic, palmitic, stearic and oleic acid etc. . . . Alkyl phosphates or phosphoric acid esters, acid phosphate, diethanolamine phosphate, potassium cetyl phosphate. Ethoxylated carboxylic acids or polyethyleneglycol esters, PEG-n acylates. Fatty alcohol polyglycolether such as laureth-n, myreth-n, ceteareth-n, steareth-n, oleth-n. Fatty acid polyglycolether such as PEG-n stearate, PEG-n oleate, PEG-n cocoate. Monoglycerides and polyol esters. C12-C22 fatty acid mono- and di-esters of addition products of from 1 to 100 mol of ethylene oxide with polyols. Fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters, glycerol and saccharose esters such as sucro glycerides. Sorbitol and sorbitan, sorbitan mono- and di-esters of saturated and unsaturated fatty acids having from 6 to 22 carbon atoms and ethylene oxide addition products. Polysorbate-n series, sorbitan esters such as sesquiisostearate, sorbitan, PEG-(6)-isostearate sorbitan, PEG-(10)-sorbitan laurate, PEG-17-dioleate sorbitan. Glucose derivatives, C8-C22 alkyl-mono and oligo-glycosides and ethoxylated analogues with glucose being preferred as the sugar component. O/W emulsifiers such as methyl gluceth-20 sesquistearate, sorbitan stearate/sucrose cocoate, methyl glucose sesquistearate, cetearyl alcohol/cetearyl glucoside. W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated parafins, sulfonated tetraproplyne sulfonate, sodium lauryl sulfates, amonium and ethanolamine lauryl sulfates, lauyl ether sulfates, sodium laureth sulfates [Texapon N70] or sodium myreth sulfates [Texapon K14S], sulfosuccinates, aceyl isothionates, alkanolamide sulfates, taurines, methyl taurines, imidazole sulfates. Zwitterionic or amphoteric surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkylbetaine, N-alkylaminobetaines.

Examples of suitable mild surfactants as dispersing agents, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, α-olefin sulfonates, ethercarboxylic acids, alkyl oligoglucosides, fatty acid glucamides, alkylamidobetaines and/or protein fatty acid condensation products, the latter preferably being based on wheat proteins.

Non ionic surfactants such as PEG-6 beeswax (and) PEG-6 stearate (and) polyglyceryl-2-isostearate [Apifac], glyceryl stearate (and) PEG-100 stearate. [Arlacel 165], PEG-5 glyceryl stearate [arlatone 983 S], sorbitan oleate (and) polyglyceryl-3 ricinoleate. [Arlacel 1689], sorbitan stearate and sucrose cocoate [arlatone 2121], glyceryl stearate and laureth-23 [Cerasynth 945], cetearyl alcohol and ceteth-20 [Cetomacrogol Wax], cetearyl alcohol and colysorbate 60 and PEG-150 and stearate-20 [Polawax GP 200, Polawax NF], cetearyl alcohol and cetearyl polyglucoside [Emulgade PL 1618], cetearyl alcohol and ceteareth-20 [Emulgade 1000NI, Cosmowax], cetearyl alcohol and PEG-40 castor oil [Emulgade F Special], cetearyl alcohol and PEG-40 castor oil and sodium cetearyl sulfate [Emulgade F], stearyl alcohol and steareth-7 and steareth-10 [Emulgator E 2155], cetearyl alcohol and szeareth-7 and steareth-10 [Emulsifying wax U.S.N.F], glyceryl stearate and PEG-75 stearate [Gelot 64], propylene glycol ceteth-3 acetate [Hetester PCS], propylene glycol isoceth-3 acetate [Hetester PHA], cetearyl alcohol and ceteth-12 and oleth-12 [Lanbritol Wax N 21], PEG-6 stearate and PEG-32 stearate [Tefose 1500], PEG-6 stearate and ceteth-20 and steareth-20 [Tefose 2000], PEG-6 stearate and ceteth-20 and glyceryl stearate and steareth-20 [Tefose 2561], glyceryl stearate and ceteareth-20 [Teginacid H, C, X].

Anionic emulsifiers such as PEG-2 stearate SE, glyceryl stearate SE [Monelgine, Cutina KD], propylene glycol stearate [Tegin P], cetearyl Alcohol and Sodium cetearyl sulfate [Lanette N, Cutina LE, Crodacol GP], cetearyl alcohol and sodium lauryl sulfate [Lanette W], trilaneth-4 phopshate and glycol stearate and PEG-2 stearate [Sedefos 75], glyceryl stearate and sodium lauryl Sulfate [Teginacid Special]. Cationic acid bases such as cetearyl alcohol and cetrimonium bromide.

The specific dispersing agents may be used in an amount of, for example, from 1 to 30% by weight, especially from 2 to 20% by weight and preferably from 3 to 10% by weight, based on the total weight of the composition.

Useful solvents are water, brine, (poly-)ethylene glycol, glycerol or cosmetically acceptable oils. Other useful solvents are disclosed below in the sections entitled “Esters of fatty acids”, “Natural and synthetic triglycerides, including glyceryl esters and derivatives”, “Pearlescent waxes”, “Hydrocarbon oils” and “Silicones or siloxanes”.

The micronised sparingly soluble organic compounds so obtained usually have an average particle size from 0.02 to 2 micrometres, preferably from 0.03 to 1.5 micrometres and more especially from 0.05 to 1.0 micrometres.

Most preferred dispersing agents (b) are sodium alkyl sulfates or sodium alkyl ether sulfates, such as sodium laureth sulfate [Texapon N70 from Cognis] or sodium myreth sulfate [Texapon K14 S from Cognis].

The aqueous dispersion used in the present invention generally comprises

30-60, preferably 35 to 55 parts of the sparingly soluble organic micronized substance;
2-20, preferably 2 to 20 parts of the dispersing agent;
0.1-1 part, preferably 0.1 to 0.5 parts of a thickening agent (for example xanthan gum); and
20-68 parts of water;

The sunscreen composition according to the invention, that is ID, is for example obtained by grinding an insoluble organic UV absorber in the presence of an aid which aid is selected from the group consisting of decyl gluocoside, polyglyceryl-10 laurate, sodium myreth sulfate and sodium stearoyl glutamate.

Any known process suitable for the preparation of microparticles can be used for the preparation of the micronised UV absorbers, for example wet-milling, wet-kneading, spray-drying from a suitable solvent, by the expansion according to the RESS process (Rapid Expansion of Supercritical Solutions), by reprecipitation from suitable solvents, including supercritical fluids (GASR process=Gas Anti-Solvent Recrystallization/PCA process=Precipitation with Compressed Anti-solvents).

The micronized UV absorbers of component (B) or more specifically b₁ so obtained usually have an average particle size from 0.02 to 2, preferably from 0.03 to 1.5, and more especially from 0.05 to 1.0 micrometer.

The micronizable UV absorbers according to component (B) or more specifically ID, can also be used as dry substrates in powder form.

These non-micronized UV absorbers may be oil soluble such as the b₃ group defined above.

These non-micronized UV absorbers may be water soluble such as the b₅ group defined above.

UV Absorber b₂

The UV absorber b₂ group is presently defined as Bis-ethylhexyloxyphenol methoxyphenyl Triazine.

Bis-ethylhexyloxyphenol is oil soluble.

UV Absorber b₃

These organic UV absorber are substantially oil soluble. They are not micronized nor are they inorganic.

The b₃ oil soluble organic UV absorbers is selected from the group of UV absorbers consisting of Butyl Methoxydibenzoylmethane (BMBM), Oxybenzone, Sulisobenzone, Bis-Ethylhexyloxyphenol Methoxyphenyl Triazine (BEMT) Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, Diethylamino Hydroxy Benzoyl Hexyl Benzoate (DHHB) and mixtures thereof;

UV Absorber b₄

Representative inorganic sunscreens of component (B) or more specifically b₄ include pigments, or alternatively nanopigments (mean size of the primary particles: generally between 5 nm and 100 nm, preferably between 10 nm and 50 nm) formed from coated or uncoated metal oxides, such as, for example, titanium oxide (amorphous or crystalline in the rutile and/or anatase form), iron oxide, zinc oxide, zirconium oxide or cerium oxide nanopigments, which are all known in the art as UV sunscreens. Conventional coating agents are, furthermore, alumina and/or aluminium stearate. Such nanopigments formed from coated or uncoated metal oxides are disclosed in particular in EP 518 772 and EP 518 773.

UV Absorber b₅

Representative water-soluble organic UV absorbers are selected from the group consisting of

Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, PEG-25 PABA and mixtures thereof.

The UV screening agent of component (B) or more specifically the total weight of b₁, b₂, b₃, b₄ or b₅ in combination with formula (I) or selective formula (Ia) is present in the sunscreen composition in amounts from about 0.01 weight % to about 50 weight % based on the weight of the total sunscreen composition. Additionally, the UV screening agent of component (B) or more specifically the total weight of b₁, b₂, b₃, b₄ or b₅ in combination with formula (1) or selective formula (Ia) is present in the sunscreen composition in amounts from about 0.1 weight % to about 30 weight % based on the weight of the total sunscreen composition. Typically, UV screening agent of component (B) or more specifically b₁, b₂, b₃, b₄ or b₅ in combination with formula (1) or selective formula (Ia) is present in the sunscreen composition in amounts from about 1 weight % to about 20 weight % based on the weight of the total composition. Typically, UV screening agent of component (B) or more specifically b₁, b₂, b₃, b₄ or b₅ in combination with formula (I) or selective formula (Ia) is present in the sunscreen composition in amounts from about 1 weight % to about 5 weight % based on the weight of the total composition.

To clarify the total weight % of component B) or the sum of b1, b2, b3, b4 and b5 may be as much as about 0.01 to about 50 wt. %, about 0.1 to about 30 wt. %, about 1 to about 25 wt. % of the total sunscreen composition.

Typically, sunscreen formulations contain compositions of several UVA, UVB or broad-spectrum sunscreen actives: organic that are oil or water soluble, inorganic or organic particulates.

The term “effective amount” means for example the amount necessary to achieve the desired effect.

For the random copolymers of component A) formula (I) or selective formula (Ia), u+v+w+x+y+z=100 weight percent relative to the total weight of the terpolymer.

The random terpolymers of component (A) formula (I) according to the instant invention are derived from at least three different monomers. Another aspect of the instant invention is the random terpolymers of component (A) formula (I) are derived from at least four different monomers.

The random terpolymers of component (A) formula (I) or selective formula (Ia) can be used in conjunction with other polymers or copolymer in a sunscreen formulation; for example, the polymers listed in U.S. Pat. No. 6,409,998 and/or in US 2006/0104923.

Another embodiment of the instant invention for component A) formula (I) is that y is from about 0.1% to about 35% by weight based on the total weight of the terpolymer. Another embodiment of the instant invention for component A) formula (I) is that y is from about 1% to about 30% by weight based on the total weight of the terpolymer. Another embodiment of the instant invention for component A) formula (I) is that y is from about 5% to about 20% by weight based on the total weight of the terpolymer.

“y” for selective formula (Ia) is from about 1 to about 30%, preferably about 5 to about 20 and most preferably about 6 to about 10% by weight of the terpolymer.

Another embodiment of the instant invention for component A) formula (I) is that v is from about 5% to about 70% by weight based on the total weight of the terpolymer. Another embodiment of the instant invention for component A) formula (I) is that v is from about 5% to about 60% by weight based on the total weight of the terpolymer. Another embodiment of the instant invention for component A) formula (I) is that v is from about 10% to about 60% by weight based on the total weight of the terpolymer.

“v” for the selective formula (Ia) is from about 5% to about 75% by weight of the terpolymer, preferably about 5 to about 50, most preferably about 8 to about 20% by weight of the terpolymer.

Another embodiment of the instant invention for component A) formula (I) is that u is from about 5% to about 75% by weight based on the total weight of the terpolymer. Another embodiment of the instant invention for component A) formula (I) is that u is from about 5% to about 65% by weight based on the total weight of the terpolymer. Another embodiment of the instant invention for component A) formula (I) is that u is from about 5% to about 60% by weight based on the total weight of the terpolymer.

“u” for the selective formula (Ia) is from about 20% to about 80%, preferably about 30% to about 75% and most preferably about 40 to about 75% by weight of the terpolymer.

Another embodiment of the instant invention for component A) formula (I) is that z is from about 0.1% to about 50% by weight based on the total weight of the terpolymer. Another embodiment of the instant invention for component A) formula (I) is that z is from about 1% to about 50% by weight based on the total weight of the terpolymer. Another embodiment of the instant invention for component A) formula (I) is that z is from about 1% to about 40% by weight based on the total weight of the terpolymer.

“z” for the selective formula (Ia) is from about 1% to about 40%, preferably about 2% to about 15% and most preferably about 3% to about 10% by weight of the terpolymer.

Another embodiment of the instant invention for component A) formula (I) is that x is from about 1% to about 40% by weight based on the total weight of the terpolymer. Another embodiment of the instant invention for component A) formula (I) is that x is from about 1% to about 30% by weight based on the total weight of the terpolymer. Another embodiment of the instant invention for component A) formula (I) is that x is from about 5% to about 25% by weight based on the total weight of the terpolymer.

“x” for the selective formula (Ia) is from about 5% to about 25%, preferably about 6% to about 20%, most preferably about 8% to about 15% by weight of the terpolymer.

Another embodiment of the instant invention for component A) formula (I) is that w is from about 0.1% to about 45% by weight based on the total weight of the terpolymer. Another embodiment of the instant invention for component A) formula (I) is that w is from about 1% to about 40% by weight based on the total weight of the terpolymer.

Another embodiment of the instant invention for component A) formula (I) is that w is from about 5% to about 30% by weight based on the total weight of the terpolymer.

Another embodiment of the instant invention for component A) formula (I) is that M is derived from at least one monomer of formula (II)

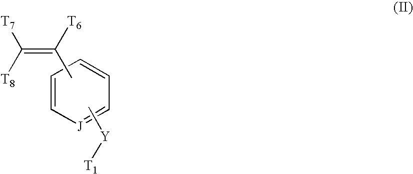

wherein T6, T7, and T8 are methyl, ethyl or hydrogen; Y is a direct bond; T1 is hydrogen or C1-C4 alkyl; and J is a carbon atom.

Another embodiment of the instant invention for component A) formula (I) is that M is derived from at least one monomer of formula (II)

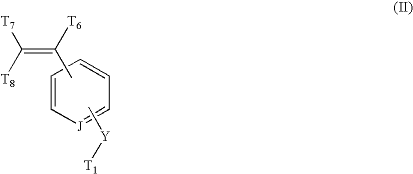

wherein T6, T7, and T8 are methyl or hydrogen; Y is a direct bond; T1 is hydrogen, methyl, or ethyl; and J is a carbon atom.

Another embodiment of the instant invention for component A) formula (I) is that M is derived from at least one monomer selected from the group consisting of styrene, alpha-methylstyrene, 2-vinyltoluene, 3-vinyltoluene, 4-vinyltoluene, ethylvinylbenzene and mixtures thereof.

Another embodiment of the instant invention for component A) formula (I) is T, D, and E are independently derived from at least one monomer of formula (III)

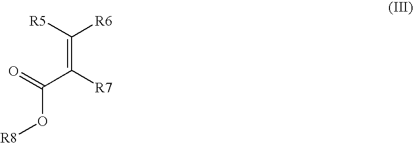

wherein R5, R6 and R7 may be the same or different and represent hydrogen or C1-C12 alkyl;

R8 is C1-C18 alkyl, or C6-C15 cycloalkyl; said substituted alkyl, or said cycloalkyl may also be substituted by one or more —OH and/or NH2 groups; said alkyl or said cycloalkyl may be interrupted by one or more —O— groups and/or —N(H)— groups.

Another embodiment of the instant invention for component A) formula (I) is T, D, and E are independently derived from at least one monomer selected from the group consisting of methyl (meth)acrylate, ethyl (meth)acrylate, propyl (meth)acrylate, butyl (meth)acrylate, isobutyl (meth)acrylate, hexyl (meth)acrylate, cyclohexyl (meth)acrylate, 2-ethylhexyl (meth)acrylate, octyl (meth)acrylate, decyl (meth)acrylate, dodecyl (meth)acrylate, dimethyl aminoethyl (meth)acrylate, isobornyl (meth)acrylate, stearyl (meth)acrylate, behenyl (meth)acrylate, polypropylene glycol mono(meth)acrylate, glycidyl (meth)acrylate, polyethylene glycol mono(meth)acrylate, EO-PO-mono(meth)acrylate and mixtures thereof. The parentheses indicate that the monomers of formula (III) are esters based on either methacrylic acid or acrylic acid.

Another embodiment of the instant invention is random terpolymers of component (A) formula (I) that consist of a polymer chain having attached thereto a monomer derived from G containing heterocyclic groups with basic nitrogen atoms. Such a chain can be obtained either by polymerizing-in compounds containing both a vinyl and such a heterocyclic group, or by later attaching a heterocyclic group to the polymer chain containing corresponding reactive groups.

Preferred are heterocyclic groups with basic nitrogen groups having a pKa value of 2 to 14, more in particular 5 to 14 and most preferably 5 to 12. These pKa values relate to the measurement thereof at 25C in a 0.01 molar concentration in water. These basic groups impart to the random terpolymers according to the invention a basicity. These basic groups allow the random terpolymers to form organic and/or inorganic salts too. The random terpolymers can therefore be used in the form of such salts.

These salts are obtained by neutralization of the polymer with organic acids, e.g., aromatic acids having not more than 25 carbon atoms or aliphatic and cycloaliphatic acids having not more than 22 carbon atoms. Preference is given to salts of the polymer with organic monocarboxylic acids. Inorganic acids are, for example, hydrochloric acid, hydrobromic acid, sulfurous acid, sulfuric acid, and the like.

Suitable compounds of component b formula (I) G to be polymerized-in are selected from the group consisting of vinylimidazole, 2-vinylpyridine, 4-vinylpyridine, 2-methyl-N-vinylimidazole, vinylpyrrolidone, vinylcarbazole and mixtures thereof.

Suitable compounds containing at least one basic nitrogen atom and capable of being attached to a polymer chain of formula (I) G are described in, among others, EP-A 154,678.

Suitable compounds containing at least one basic nitrogen atom and capable of being attached to a polymer chain of formula (I) G are selected from the group consisting of 1-(2-hydroxyethyl)-pyrrolidine, 2-(1-pyrrolidyl)-ethylamine, 2-(1-piperidyl)-ethylamine, 1-(2-hydroxyethyl)-piperidine, 1-(2-aminopropyl)-piperidine, N-(2-hydroxyethyl)-hexamethylenimine, 4-(2-hydroxyethyl)-morpholine, 2-(4-morpholinyl)-ethylamine, 4-(3-aminopropyl)-morpholine, 1-(2-hydroxyethyl)-piperazine, 1-(2-aminoethyl)-piperazine, 1-(2-hydroxyethyl)-2-alkylimidazoline, 1-(3-aminopropyl)-imidazole, (2-aminoethyl)-pyridine, (2-hydroxyethyl)-pyridine, (3-hydroxypropyl)-pyridine, (hydroxymethyl)-pyridine, N-methyl-2-hydroxy-methyl-piperidine, 1-(2-hydroxyethyl)-imidazole, 2-amino-6-methoxybenzothiazole, 4-aminomethyl-pyridine, 4-amino-2-methoxypyrimidine, 2-mercaptopyrimidine, 2-mercapto-benzimidazole, 3-mercapto-1,2,4-triazole, 3-amino-1,2,4-triazole, 2-isopropyl-imidazole, 2-ethyl-imidazole, 4-methyl-imidazole, 2-methyl-imidazole, 2-ethyl-4-methyl-imidazole, 2-phenyl-imidazole, 4-nitro-imidazole and mixtures thereof.

Another embodiment of the instant invention for component A) formula (I) H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bisphenylene diisocyanate, 4,4′-bis(2-methoxyisocyanatophenyl)methane, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, m-tetramethylxylylene diisocyanate and mixtures thereof.

Another embodiment of the instant invention for component A) formula (I) is that H is derived from at least one monomer selected from the group consisting of toluene diisocyanate, 4,4′-diphenylmethane diisocyanate, tolidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bisphenylene diisocyanate, 4,4′-bis(2-methoxyisocyanatophenyl)methane, 4,4′-diisocyanatodiphenyl ether, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, 4,4′-dicyclohexylmethane diisocyanate, isophorone diisocyanate, 3-isocyanatomethyl-3,5,5-trimethylcyclohexyl diisocyanate, lysine methyl ester diisocyanate, m-tetramethylxylylene diisocyanate and mixtures thereof.

The random terpolymers of formula (I) according to the instant invention may be crosslinked by multifunctional monomers. These multifunctional monomers are selected from the group consisting of divinyl benzene, trivinylbenzene, divinyltoluene, divinylpyridine, divinylnaphthalene divinylxylene, ethyleneglycol di(meth)acrylate, trimethylolpropane tri(meth)acrylate, diethyleneglycol divinyl ether, trivinylcyclohexane, allyl (meth)acrylate, diethyleneglycol di(meth)acrylate, propyleneglycol di(meth)acrylate, 2,2-dimethylpropane-1,3-di(meth)acrylate, 1,3-butylene glycol di(meth)acrylate, 1,4-butanediol di(meth)acrylate, 1,6-hexanediol di(meth)acrylate, tripropylene glycol di(meth)acrylate, triethylene glycol di(meth)acrylate, tetraethylene glycol di(meth)acrylate, polyethylene glycol di(meth)acrylates, polyethylene glycol 200 di(meth)acrylate, polyethylene glycol 600 di(meth)acrylate, ethoxylated bisphenol A di(meth)acrylate, poly(butanediol) di(meth)acrylate, pentaerythritol tri(meth)acrylate, trimethylolpropane triethoxy tri(meth)acrylate, glyceryl propoxy tri(meth)acrylate, pentaerythritol tetra(meth)acrylate, dipentaerythritol monohydroxypenta(meth)acrylate, divinyl silane, trivinyl silane, dimethyl divinyl silane, divinyl methyl silane, methyl trivinyl silane, diphenyl divinyl silane, divinyl phenyl silane, trivinyl phenyl silane, divinyl methyl phenyl silane, tetravinyl silane, dimethyl vinyl disiloxane, poly(methyl vinyl siloxane), poly(vinyl hydro siloxane), poly(phenyl vinyl siloxane), and mixtures thereof.

The weight-average molecular weight of the random terpolymer of component (A) formula (I) exhibits a weight-average molecular weight of about 500 Daltons to about 1,000,000 Daltons. In another aspect of the instant invention, the weight-average molecular weight of the random terpolymer of component (b) formula (I) exhibits a weight-average molecular weight of about 500 Daltons to about 500,000 Daltons. In yet another aspect of the instant invention, the weight-average molecular weight of the random terpolymer of component (b) formula (I) exhibits a weight-average molecular weight of about 500 Daltons to about 100,000 Daltons. In still another aspect of the instant invention, the weight-average molecular weight of the random terpolymer of component (b) formula (I) exhibits a weight-average molecular weight of about 1000 Daltons to about 75,000 Daltons.

The weight-average molecular weight of the selective terpolymer of formula (Ia) ranges for example from about 5,000 to about 50,000, preferably about 10,000 to about 40,000 and most preferably about 15,000 to about 30,000 Daltons.

The random terpolymer of component (A) formula (I) is present in the sunscreen composition in amounts from about 0.01 weight % to about 50 weight % based on the weight of the total composition. In another aspect of the instant invention, the random terpolymer of component (b) formula (I) is present in the sunscreen composition in amounts from about 0.1 weight % to about 25 weight % based on the weight of the total composition. In still another aspect of the instant invention, the random terpolymer of component (b) formula (I) is present in the sunscreen composition in amounts from about 0.1 weight % to about 10 weight % based on the weight of the total composition.

The selective formula (Ia) may be present for example in an amount from about 0.01 weight percent to about 10 weight percent based on the weight of the total composition, more preferably in an amount from about 0.1 weight percent to about 8 weight percent based on the weight of the total composition, and most preferably in an amount from about 0.1 weight percent to about 5 weight percent based on the weight of the total composition.

Another embodiment of the instant invention are random terpolymers of component (A) formula (I) or selective formula (Ia) that contain less than 250 ppm of residual monomers. Another embodiment of the instant invention are random terpolymers of component (A) formula (I) or selective formula (Ia) that contain less than 200 ppm of residual monomers. Another embodiment of the instant invention are random terpolymers of component (A) formula (I) or selective formula (Ia) that contain less than 100 ppm of residual monomers. Another embodiment of the instant invention are random terpolymers of component (A) formula (I) or selective formula (Ia) that contain less than 50 ppm of residual monomers. Another embodiment of the instant invention are random terpolymers of component (A) formula (I) or selective formula (Ia) that contain less than 5 ppm of residual monomers.

The random terpolymers of formula (I) or the selective formula (Ia) according to the instant invention are water-dispersible and can be distributed throughout the aqueous phase or the oil phase of the instant compositions or formulations.

The random terpolymers of component (b) formula (I) or selective formula (Ia) can be prepared in the conventional manner, e.g., by mass or solution polymerization. The polymerization in a solvent is preferred in view of the controllability of the polymerization and the viscosity of the final product. Suitable solvents are DMSO, THF, DMF, ethyl, propyl, butyl, acetate, benzene, toluene, xylene, N-butanol, isobutanol, isopropanol, MEK, MIRK, acetone, etc.

The monomers are preferably polymerized using a radical reaction, by addition of peroxides, optionally in the presence of redox systems.

The polymerization time of the random terpolymer of component (A) formula (I) or selective formula (Ia) depends on the temperature and the desired final product properties but is preferably within the range of from 0.5 to 10 hours at temperatures ranging from about 50C to about 190C. The polymerization can be carried out continuously, discontinuously or semicontinuously. If it is preferred to obtain a polymer chain having random distribution of monomers, all of the monomers together will be preferably added to the reaction mixture. This may be done in one portion or in the course of time.

On the basis of the reactivity of the monomers, which is known, a skilled artisan can control the polymerization so as to obtain the desired distribution.

The sunscreen compositions according to the invention both formula I and Ia may also contain agents for tanning and/or for artificial tanning of the skin (self-tanning agents), such as, for example, dihydroxyacetone (DHA).

The sunscreen compositions according to the invention may also contain agents for lightening or brightening of the skin, such as, for example, kojic acid, or arbutin.

The compositions of the invention may further comprise, cosmetically acceptable ingredients and adjuvants selected, in particular, from among fatty substances, organic solvents, thickeners, demulcents, opacifiers, colorants, effect pigments, stabilizers, emollients, antifoaming agents, moisturizing agents, antioxidants, vitamins, peptides, amino acids, botanical extracts, particulates, perfumes, preservatives, polymers, fillers, sequestrants, propellants, alkalinizing or acidifying agents or any other ingredient customarily formulated into cosmetics, in particular for the production of anti-sun/sunscreen compositions.

For example, sunscreen compositions comprising the component A) which may be formula (I) or select formula (Ia) and the various UV absorbers may further contain components selected from the group consisting of emollients, skin moisturisers, skin tanning accelerators, antioxidants, emulsion stabilisers, thickening agents, moisture retention agents, film formers, preservatives, perfumes, photostabilisers and colourants.

Especially preferred photostabilisers for example, may be selected from the group consisting of: Tris(Tetramethylhydroxypiperidinol) Citrate, Benzotriazolyl Dodecyl p-Cresol, Butyloctyl Salicylate, Diethylhexyl 2,6-Naphthalate and Polyester-8, Diethylhexyl Syringylidenemalonate.

The fatty substances may be an oil or a wax or mixtures thereof, and they also comprise fatty acids, fatty alcohols and esters of fatty acids. The oils may be selected from among animal, vegetable, mineral or synthetic oils and, in particular, from among liquid paraffin, paraffin oil, silicone oils, volatile or otherwise, isoparaffins, polyolefins, fluorinated or perfluorinated oils. Likewise, the waxes may be animal, fossil, vegetable, mineral or synthetic waxes which are also known per se.

Exemplary organic solvents include the lower alcohols and polyols.

Of course, one skilled in this art will take care to select this or these optional additional compounds and/or their quantities such that the advantageous properties, in particular the resistance to water, the stability, which are intrinsically associated with the sunscreen compositions in accordance with the invention are not, or not substantially, altered by the addition(s) envisaged.

The sunscreen compositions of the invention may be formulated according to techniques well known to this art, in particular those suited for the preparation of emulsions of the oil-in-water or water-in-oil type.

The subject sunscreen compositions may be provided, in particular, in the form of a simple or complex (O/W, W/O, O/W/O or W/O/W) emulsion such as a cream, a milk, a gel or a gel cream, of a powder, a lotion, an ointment, a solid stick and may optionally be packaged as an aerosol and provided in the form of a foam, mousse or spray.

The sunscreen composition may be formulated as an Oil-in-Water (O/W), Water-in-Oil (W/O), Oil-in-Water-Oil (O/W/O), Water-Oil-in-Water (W/O/W), PIT emulsions or micro-emulsions containing the formula (I) or the selective formula (Ia) along with the UV absorber or disclosed combinations of absorbers, b₁, b₂, b₃, b₄ or b₅.

The sunscreen composition may be formulated as gels, lotions, milks, sprays, alcoholic or aqueous/alcoholic lotions, aerosol, wax/fat compositions, stick preparations, powders, tablets, foams or ointments containing the formula (I) or the selective formula (Ia) along with the UV absorber or disclosed combinations of absorbers b₁, b₂, b₃, b₄ or b₅.

The sunscreen compositions may be for example a rinse-off or leave-on sunscreen composition containing the formula (I) or the selective formula (Ia) along with the UV absorber or disclosed combinations of absorbers b₁, b₂, b₃, b₄ or b₅.

When an emulsion is provided, the aqueous phase thereof may comprise a nonionic vesicular dispersion prepared according to known techniques (Bangham, Standish and Watkins, J. Mol. Biol., 13, 238 (1965), FR-2,315,991 and FR-2,416,008).

The sunscreen compositions according to the invention may be formulated for protecting the human epidermis or the hair against the damaging effects of ultraviolet radiation, as an anti-sun composition or as a makeup product.

When the sunscreen compositions according to the invention are formulated for protecting the human epidermis against UV rays, or as anti-sun/sunscreen compositions, same may be provided in the form of a suspension or a dispersion in solvents or fatty substances, in the form of a nonionic vesicular dispersion or, alternatively, in the form of an emulsion, preferably of the oil-in-water type, such as a cream or a milk, in the form of an ointment, a gel, a gel cream, a solid stick, a powder, a stick, an aerosol foam or a spray.

When the sunscreen compositions according to the invention are formulated for protecting the hair against UV rays, same may be provided in the form of a shampoo, a body wash, a lotion, a gel, an alcohol-based system, an emulsion, a nonionic vesicular dispersion and may constitute, for example, a rinse-off composition to be applied before or after shampooing, before or after dyeing or bleaching, before, during or after permanent-waving or hair straightening, a hair-styling or treatment lotion or gel, a lotion or gel for blow drying or hair setting, a composition for permanent waving or straightening, dyeing or bleaching the hair.

When the subject compositions are formulated as makeup products for the eyelashes, the eyebrows or the skin, such as a treatment cream for the epidermis, foundation, lipstick, eyeshadow, blusher, mascara or eyeliner, same may be provided in a solid or pasty, anhydrous or aqueous form, such as oil-in-water or water-in-oil emulsions, nonionic vesicular dispersions or alternatively suspensions.

For example, for the anti-sun formulations in accordance with the invention which have a carrier, vehicle or diluent of the oil-in-water emulsion type, the aqueous phase (comprising in particular the hydrophilic screening agents), generally constitutes from 50% to 95% by weight, preferably from 70% to 90% by weight, relative to the total weight of the formulation, the oily phase (comprising in particular the lipophilic screening agents), from 5% to 50% by weight, preferably from 10% to 30% by weight, relative to the total weight of the formulation, and the (co)emulsifier(s) from 0.5% to 20% by weight, preferably from 2% to 10% by weight, also relative to the total weight of the formulation.

As indicated above, the present invention thus features formulating the subject emulsions for the production of cosmetic compositions for protecting the skin and/or the hair against ultraviolet radiation, in particular solar radiation.

The sunscreen composition of the instant invention may further comprise a fragrance. The term “perfume” or “fragrance” as used herein refers to odoriferous materials which are able to provide a pleasing fragrance to fabrics, and encompasses conventional materials commonly used in cosmetic compositions to counteract a malodor in such compositions and/or provide a pleasing fragrance thereto. The perfumes are preferably in the liquid state at ambient temperature, although solid perfumes are also useful, particularly cyclodextrin/perfume inclusion complexes for controlled release. Included among the perfumes contemplated for use herein are materials such as aldehydes, ketones, esters and the like which are conventionally employed to impart a pleasing fragrance to liquid and solid personal care or cosmetic compositions. Naturally occurring plant and animal oils are also commonly used as components of perfumes. Accordingly, the perfumes useful for the present invention may have relatively simple compositions or may comprise complex mixtures of natural and synthetic chemical components, all of which are intended to provide a pleasant odor or fragrance when applied to fabrics. The perfumes used in personal care or cosmetic compositions are generally selected to meet the normal requirements of odor, stability, price and commercial availability. The term “fragrance” is often used herein to signify a perfume itself, rather than the aroma imparted by such perfume.

The present invention is directed to a method of increasing the sun protection factor of a sunscreen composition wherein said method comprises incorporating into said composition an effective amount of at least one random terpolymer according to formula (I) defined above or the selective terpolymer of formula (Ia) as defined above.

The present invention is directed to a method of improved UV protection of mammalian hair and/or skin from the damaging effects of UV radiation wherein said method comprises applying to said skin and/or said hair an effective amount of a sunscreen composition comprising

(A) at least one random terpolymer of formula (I) or the random terpolymer of selective formula (Ia);
(B) at least one UV screening agent;

and

(C) optionally, other cosmetically acceptable ingredients,

The present invention is directed to a cosmetic or dermatological composition comprising a random terpolymer of formula (I) or the selective random terpolymer of formula (Ia)

and
(C) other cosmetically acceptable ingredients.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

Some of the solvents used for the synthesis of the instant copolymers may not be suitable for human physiological conditions. Once the synthesis is completed, the solvents can be removed and/or replaced with solvents that are more cosmetically acceptable.

### Example 1

### Random Terpolymer

In a reaction flask with reflux condenser suitable for polymerization are dissolved in 9.86 g xylene and 4.93 g methoxypropyl acetate 2.84 g vinyl toluene, 4.55 g isobutyl methacrylate, 7.36 g 2-ethylhexyl acrylate, 5.20 g 2-hydroxyethyl methacrylate, 1.80 g polyethylene glycol monomethacrylate having a molecular weight of approximately 400 and 0.44 g ditertiary butyl peroxide. Polymerization is effected at the boiling point of the mixture while stirring and introducing an inert gas. At the end of the polymerization, 9.79 g isophorone diisocyanate are dissolved in 16.58 g isobutyl acetate and 16.58 g methoxypropyl acetate, and the remaining free NCO groups are then converted with 3.60 g polyethylene glycol monomethacrylate having a molecular weight of approximately 400 and 4.51 g 1-(3-aminopropyl)imidazole.

The solid content is then adjusted to 40% by weight with butylacetate.

According to formula (I), component M is vinyl toluene and y is 7.2 weight percent relative to the total weight of the terpolymer; component T is a mixture of isobutyl methacrylate and 2-ethylhexyl acrylate and v is 30.1 weight percent relative to the total weight of the terpolymer; component D is polyethylene glycol monomethacrylate and u is 13.6 weight percent relative to the total weight of the terpolymer; component E is 2-hydroxyethyl methacrylate and z is 13.1 weight percent relative to the total weight of the terpolymer; component G is 1-(3-aminopropyl)imidazole and x is 11.4 weight percent relative to the total weight of the terpolymer; and component H is isophorone diisocyanate and w is 24.7 weight percent relative to the total weight of the terpolymer.

### Example 2

### Random Terpolymer

In the manner comparable with Example 1, 3.54 g vinyl toluene, 5.69 g isobornyl methacrylate, 9.20 g 2-ethylhexyl methacrylate, 7.15 g hydroxy ethyl methacrylate, and 1.28 g ditertiary butylperoxide dissolved in 11.94 g xylene and 5.97 g methoxypropyl acetate are polymerized.

Subsequently, 12.23 g isophorone diisocyanate dissolved in 20.36 g butylacetate and 20.36 g methoxypropyl acetate are added. The remaining free NCO groups are then converted with 4.50 g polyethylene glycol monomethacrylate having a molecular weight of approximate 400 and 3.78 g 3-amino-1,2,4-triazole in 11.34 g N-methylpyrrolidone.

The solid content is then adjusted to 40% by weight with butylacetate.

According to formula (I), component M is vinyl toluene and y is 7.7 weight percent relative to the total weight of the terpolymer; component T is a mixture of isobornyl methacrylate and 2-ethylhexyl methacrylate and v is 32.3 weight percent relative to the total weight of the terpolymer; component D is polyethylene glycol monomethacrylate and u is 9.8 weight percent relative to the total weight of the terpolymer; component E is 2-ethylhexylmethacrylate and z is 15.5 weight percent relative to the total weight of the terpolymer; component G is 3-amino-1,2,4-triazole and x is 8.2 weight percent relative to the total weight of the terpolymer; and component H is isophorone diisocyanate and w is 26.5 weight percent relative to the total weight of the terpolymer.

### Example 3

### Random Terpolymer

In the manner described in Example 1, 6.66 g isobornyl methacrylate, 5.46 g cyclohexyl methacrylate, 6.40 g n-butylacrylate, and 7.85 g 2-hydroxyethyl methacrylate are polymerized with 1.28 g ditertiary butyl peroxide dissolved in 11.98 g xylene and 5.99 g methoxypropyl acetate. To this polymer containing hydroxyl groups, 12.23 g isophorone diisocyanate dissolved in 20.4 g butylacetate and 20.40 g methoxypropyl acetate are added. The free NCO groups are then converted with 4.50 g polyethylene glycol monomethacrylate and 5.54 g 2-(2-pyridyl)-ethanol.

The solid content is then adjusted to 40% by weight with xylene.

According to formula (I), component T is a mixture of isobornyl methacrylate and cyclohexyl methacrylate and v is 24.9 weight percent relative to the total weight of the terpolymer; component D is polyethylene glycol monomethacrylate and u is 9.3 weight percent relative to the total weight of the terpolymer; component E is a mixture of 2-hydroxyethyl methacrylate and butylacrylate and z is 29.3 weight percent relative to the total weight of the terpolymer; component G is 2-(2-pyridyl)-ethanol and x is 11.4 weight percent relative to the total weight of the terpolymer; and component H is isophorone diisocyanate and w is 25.1 weight percent relative to the total weight of the terpolymer.

### Example 4

### Random Terpolymer

In the manner described in Example 1, the polymerization of 3.78 g vinyl toluene, 5.69 g isobutyl methacrylate, 7.38 g 2-ethyl hexyl methacrylate, 7.97 g stearyl methacrylate, 4.55 g glycidyl methacrylate and 0.59 g ditertiary butylperoxide is effected in 14.98 g xylene and 4.99 g methoxypropyl acetate.

At the end of the polymerization, 24.97 g butylacetate and 4.01 g 1-(3-aminopropyl)imidazole are added to the polymer.

According to formula (I), component M is vinyl toluene and y is 11.3 weight percent relative to the total weight of the terpolymer; component T is a mixture of isobutyl methacrylate and 2-ethylhexyl methacrylate and v is 39.1 weight percent relative to the total weight of the terpolymer; component D is stearyl methacrylate and u is 23.9 weight percent relative to the total weight of the terpolymer; component E is glycidyl methacrylate and z is 13.6 weight percent relative to the total weight of the terpolymer; and component G is 1-(3-aminopropyl)imidazole and x is 12.0 weight percent relative to the total weight of the terpolymer.

### Example 5

### Random Terpolymer

In the manner described in Example 1, the polymerization of 6.66 g isobornyl methacrylate, 5.46 g cyclohexyl methacrylate, 9.96 g stearyl methacrylate, 9.22 g 2-ethyl hexyl methacrylate, 5.69 g glycidyl methacrylate and 0.74 g ditertiary butylperoxide is effected in 18.86 g xylene and 6.29 g methoxypropyl acetate.

At the end of the polymerization, 18.94 g butyl acetate and 4.05 g 3-mercapto-1,2,4-triazole dissolved in 16.20 g N-methylpyrrolidone are added to the polymer.

According to formula (I), component T is a mixture of isobornyl methacrylate and cyclohexyl methacrylate and v is 29.5 weight percent relative to the total weight of the terpolymer; component D is a mixture of 2-ethylhexyl methacrylate and stearyl methacrylate and u is 46.8 weight percent relative to the total weight of the terpolymer; component E is glycidyl methacrylate and z is 13.9 weight percent relative to the total weight of the terpolymer; and component G is 3-mercapto-1,2,4-triazole and x is 9.9 weight percent relative to the total weight of the terpolymer.

### Example 6

### Random Terpolymer

In the manner described in Example 1, the polymerization of 12.0 g methyl methacrylate, 32.76 g cyclohexyl methacrylate, 35.84 g butylacrylate, 18.82 g vinyl imidazole and 2.0 g tertiary butyl perbenzoate is effected in 50.71 g xylene and 16.91 g n-butanol.

The solid content is adjusted to 40% by weight with butyl acetate.

According to formula (I), component T is methyl methacrylate and v is 12.1 weight percent relative to the total weight of the terpolymer; component D is cyclohexyl methacrylate and u is 33.0 weight percent relative to the total weight of the terpolymer; component E is butyl acrylate and z is 36.0 weight percent relative to the total weight of the terpolymer; and component G is vinyl imidazole and x is 18.9 weight percent relative to the total weight of the terpolymer.

### Example 7

### Random Terpolymer

In 15.67 g secondary butanol and 47.0 g butyl acetate are polymerized, in the manner described in Example 1: 29.97 g isobornyl methacrylate, 9.36 g styrene, 38.71 g 2-ethyl hexyl acrylate, 14.12 g vinyl imidazole, 0.62 g tertiary butyl-per-2-ethyl hexoate and 1.23 g tertiary butyl perbenzoate.

At the end of the polymerization, the solid content is adjusted to 50% by weight with butyl acetate.

According to formula (I), component M is styrene and y is 10.2 weight percent relative to the total weight of the terpolymer; component T is isobornyl methacrylate and v is 32.5 weight percent relative to the total weight of the terpolymer; component D is 2-ethylhexyl acrylate and u is 42.0 weight percent relative to the total weight of the terpolymer; and component G is vinyl imidazole and x is 15.3 weight percent relative to the total weight of the terpolymer.

### Example 8

### Random Terpolymer

In 45.08 g xylene and 22.54 g n-butanol, 12.00 g methyl methacrylate, 32.76 g cyclohexyl methacrylate, 35.84 g butyl acrylate, 18.82 g vinyl imidazole and 2.0 g tertiary butyl perbenzoate are polymerized in the manner described in Example 1.

At the end of the polymerization, the solid content is adjusted to 50% by weight by adding 33.80 g xylene.

According to formula (I), component T is methyl methacrylate and v is 12.1 weight percent relative to the total weight of the terpolymer; component D is cyclohexyl methacrylate and u is 33.0 weight percent relative to the total weight of the terpolymer; component E is butyl methacrylate and z is 36.0 weight percent relative to the total weight of the terpolymer; and component G is vinyl imidazole and x is 18.9 weight percent relative to the total weight of the terpolymer.

### Example 9

### Random Terpolymer

In 47.00 g toluene and 15.67 g n-butanol, 29.97 g isobornyl methacrylate, 9.36 g styrene, 38.71 g 2-ethyl hexyl acrylate, 14.12 g vinyl imidazole and 11.85 g tertiary butyl perbenzoate are polymerized in the manner described in Example 1.

At the end of the polymerization, a polymer solution is obtained having a solid content of 60% by weight.

According to formula (I), component M is styrene and y is 10.2 weight percent relative to the total weight of the terpolymer; component T is isobornyl methacrylate and v is 32.5 weight percent relative to the total weight of the terpolymer; component D is 2-ethylhexyl acrylate and u is 42.0 weight percent relative to the total weight of the terpolymer; and component G is vinyl imidazole and x is 15.3 weight percent relative to the total weight of the terpolymer.

### Example 10

### Random Terpolymer

In 43.75 g xylene and 14.59 g n-butanol, 23.31 g isobornyl methacrylate, 31.35 g butyl acrylate, 10.92 g styrene, 3.71 g acrylonitrile, 16.47 g vinyl imidazole and 1.72 g tertiary butyl perbenzoate are polymerized.

At the end of the polymerization, the solid content of the polymer solution is adjusted to 50% by weight by adding xylene.

According to formula (I), component M is styrene and y is 12.7 weight percent relative to the total weight of the terpolymer; component T is isobornyl methacrylate and v is 27.2 weight percent relative to the total weight of the terpolymer; component D is butylacrylate and u is 36.6 weight percent relative to the total weight of the terpolymer; component G is vinyl imidazole and x is 19.2 weight percent relative to the total weight of the terpolymer; and H is acrylonitrile and w is 4.3 weight percent relative to the total weight of the terpolymer.

### Example 11

### Random Terpolymer

In the manner described in Example 1, 19.98 g isobornyl methacrylate, 10.62 g vinyl toluene, 30.42 g 2-ethylhexyl acrylate, 6.75 g polyethylene glycol monomethacrylate, 16.38 g cyclohexyl methacrylate, 15.53 g vinyl imidazole, 0.67 g tertiary butyl peroctoate and 1.34 g tertiary butyl perbenzoate are polymerized in 50.85 g butyl acetate and 16.95 g secondary butanol.

At the end of the polymerization, the solid content of the polymer solution is adjusted to 50% by weight with butyl acetate.

According to formula (I), component M is vinyl toluene and y is 10.7 weight percent relative to the total weight of the terpolymer; component T is a mixture of isobornyl methacrylate and 2-ethylhexyl acrylate and v is 50.5 weight percent relative to the total weight of the terpolymer; component D is polyethylene glycol monomethacrylate and u is 6.8 weight percent relative to the total weight of the terpolymer; component E is cyclohexyl methacrylate and z is 16.4 weight percent relative to the total weight of the terpolymer; and component G is vinyl imidazole and x is 15.6 weight percent relative to the total weight of the terpolymer.

### Example 12

### Random Terpolymer

In 98.67 g butyl acetate and 19.74 g n-butanol, the following substances are polymerized, in the manner described in Example 1: 19.98 g isobornyl methacrylate, 10.92 g cyclohexyl methacrylate, 10.62 g vinyl toluene, 15.0 g methyl methacrylate, 6.75 g polyethylene glycol monomethacrylate, 14.12 g vinyl imidazole and 1.56 g tertiary butyl perbenzoate.

At the end of the polymerization, the solid content of the solution is adjusted to 40% by weight by adding butyl acetate.

According to formula (I), component M is vinyl toluene and y is 13.7 weight percent relative to the total weight of the terpolymer; component T is a mixture of isobornyl methacrylate and cyclohexyl methacrylate and v is 39.9 weight percent relative to the total weight of the terpolymer; component D is polyethylene glycol monomethacrylate and u is 8.7 weight percent relative to the total weight of the terpolymer; component E is methyl methacrylate and z is 19.4 weight percent relative to the total weight of the terpolymer; and component G is vinyl imidazole and x is 18.2 weight percent relative to the total weight of the terpolymer.

### Example 13

### Random Terpolymer

The following substances are polymerized randomly, similar to the manner described in Example 11 except sec-butanol is used as the solvent: 9.0 g vinyltoluene, 6.6 g 2-hydroxyethyl methacrylate, 13.2 g vinyl imidazole, 14.1 g 2-ethylhexylacrylate, and 66.9 g monomethoxypolyethylene glycol monomethacrylate. After completion of the polymerization reaction, all solvents and volatiles are removed by vacuum distillation. A polymeric melt is obtained with a molecular weight of about 15,000-20,000 Dalton as determined by Gel Permeation Chromotography (GPC).

According to formula (I), component M is vinyl toluene and y is 8.2 weight percent relative to the total weight of the terpolymer; component T is 2-ethylhexyl methacrylate and v is 12.8 weight percent relative to the total weight of the terpolymer; component D is polyethylene glycol monomethacrylate and u is 60.9 weight percent relative to the total weight of the terpolymer; component E is 2-hydroxyethyl methacrylate and z is 6.0 weight percent relative to the total weight of the terpolymer; and component G is vinyl imidazole and x is 12.0 weight percent relative to the total weight of the terpolymer.

A 50% (w/w) solution of the random terpolymer is prepared by dissolving 109.8 g of the random terpolymer synthesized above in a 109.8 g of Water.

This solution is an easy-to-handle form of the random terpolymer that is suitable for intended applications.

pH of terpolymer at 50% concentration in water is 5.6-6.0

Average Molecular weight of terpolymer of example 13: ˜15,000 to 20,000 Daltons.

Tg of example 13 terpolymer is 2.85° C.

Minimum Film Forming Temperature of example 13 terpolymer is more than 90° C.

### Example 14

### Sunscreen Composition Preparation

| Part | Trade name | INCI name | Function | % W/W* |
|---|---|---|---|---|
| A | Arlacel 165 | Glyceryl Stearate (and) | Emulsifier, non-ionic | 3.50 |
| | | PEG-100 Stearate | | |
| | Lanette 16 | Cetyl Alcohol | Emulsion Stabilizer | 1.00 |
| | Cetiol B | Dibutyl Adipate | Emollient/solvent | 5.00 |
| | Cetiol CC | Dicaprylyl Carbonate | Emollient/solvent | 5.00 |
| | Tegosoft DEC | Diethylhexyl Carbonate | Emollient/solvent | 2.00 |
| | Neo Heliopan AV | Drug name: Octinoxate | Sunscreen Active | 7.50 |
| | | INCI name: Ethylhexyl | | |
| | | Methoxycinnamate | | |
| | Neo Heliopan OS | Drug name: Octisalate | Sunscreen Active | 2.00 |
| | | INCI name: Ethylhexyl | | |
| | | Salicylate | | |
| | Parsol 1789 | Drug name: Avobenzone | Sunscreen Active | 3.00 |
| | | INCI name: Butyl | | |
| | | Methoxydibenzoylmethane | | |
| | Tinosorb ® S | Drug name (proposed): | Sunscreen Active | 1.50 |
| | | Bemotrizinol; | | |
| | | INCI name: Bis- | | |
| | | Ethylhexyloxyphenol | | |
| | | Methoxyphenyl Triazine | | |
| | Orgasol 2002 EXD | Nylon-12 | Spherical Particulate to | 1.50 |
| | NAT COS | | improve skin feel | |
| B | Water | Water | Diluent/solvent | Qs to 100.00 |
| | Keltrol CG RD | Xanthan Gum | Rheology Modifier | 0.22 |
| | Glycerin | Glycerin | Humectant | 3.00 |
| | Dissolvine NA2 | Disodium EDTA | Chelating Agent | 0.20 |
| C | Dow Corning 345 | Cyclopentasiloxane | Emollient/Solvent | 2.00 |
| | Fluid | | | |
| | Tinovis ADM | Sodium Acrylates | Rheology Modifier | 0.83 |
| | | Copolymer (and) Mineral | | |
| | | Oil (and) PPG-1 Trideceth-6 | | |
| | Tinosorb ® M | Drug name (proposed): | Sunscreen Active | 3.00 (as is) or |
| | | Bisoctrizole; | | 1.50 (active |
| | | INCI name: Methylene Bis- | | level) |
| | | Benzotriazolyl | | |
| | | Tetramethylbutylphenol | | |
| | Phenonip | Phenoxyethanol (and) | Preservative | 1.00 |
| | | Methylparaben (and) | | |
| | | Ethylparaben (and) | | |
| | | Butylparaben (and) | | |
| | | Propylparaben (and) | | |
| | | Isobutylparaben | | |
| | Sodium Hydroxide | Sodium Hydroxide (15% | pH adjustor | Qs to pH ~5.3-6.1 |
| | | aqueous solution) | | |

*The components are added to the sunscreen composition on a % weight/weight of component (as active) based on the weight of the total composition.

Combine the ingredients of part A. Heat up part A to 80° C. with mixing. Mix until uniform, and add Nylon-12 with moderate agitation.

Prepare part B: first, disperse Xanthan Gum into the water and heat up to 80° C. When uniform, add the rest of part B one by one, mix until uniform.

Add part A into part B under stirring, and then homogenize with an Ultra Turrax pos 2 for 40 sec/100 g.

Cool down under stirring, to 40° C. and add the ingredients of part C one by one in the given order. Mix until uniform. If necessary, adjust pH with aqueous solution of sodium sydroxide to 5.3-6.1

### Example 15

### Testing Protocol

The test protocol described below is used to mimic the application of the sunscreen composition to human skin and test the initial SPF and the SPF after eighty minutes of water exposure of the instant compositions.

The following laboratory equipment is used:

VITRO-SKIN® N-19, Foam block, Hydration Chamber, Powder Free Rubber Finger Cots and Glassless slide mounts are obtained from IMS, Inc. (70 Robinson Blvd, Orange, Conn., USA);
Water bath (# 05-719-7F), Corning Hotplate Stirrer (#11-497-8A), Calfarno Compact Digital Stirrer (#14-500-7), Glycerol Aqueous Solution (#AC277366-0010) are obtained from Fisher Scientific Catalog; and
Optometrics SPF 290 is obtained from Optometrics LLC. (8 Nemco Way, Stony Brook Industrial Park, Ayer, Mass., USA).

An aqueous solution of glycerin (300 g of 14.7% by weight) is prepared and poured on the bottom of the hydration chamber. The shelves are placed in the chamber that is covered with a lid. VITRO-SKIN substrate is cut into 4.1 cm×4.1 cm pieces that are placed on the shelves in a hydration chamber and hydrated for 16-22 hours prior to the tests.

Optometrics SPF 290S is turned on followed by the manufacturer's directions for instrument calibration, blank and sample measurements.

A piece of substrate is placed in a slide mount and used as a reference for the in vitro SPF measurements. Another piece of substrate is placed on a plastic-covered foam block and product application is made to the “topography” side of the substrate (the rough side). The test composition (0.033 g) is applied evenly across a 4 cm×4 cm section of the substrate, which results in an application dose of 2 mg/sq. cm and rubbed into the substrate with a finger covered with finger cot. After this, the substrate is placed on a slide mount.

The in vitro SPF measurements are made both prior to and after sample immersion in water with stirring for 80 minutes at a water temperature of 37+/−0.5C. All initial measurements are made after the 15 minute dry-down period. After water exposure, the samples are removed, air-dried for about 30 minutes, placed back in the controlled humidity chamber for 120 minutes followed by the 15 minute dry-down period. The reference slides are immersed in the water bath for the same amount of time. An Optometrics SPF 290S is used to determine UV absorbance for each formulation in the 290-400 nm wavelength range. A minimum of three consecutive measurements on three separate areas of the slide are conducted. SPF, UVA/UVB and Critical Wavelength in vitro values for each sample—before and after water immersion are recorded. The % SPF remaining after eighty minute exposure to water is calculated by:

(a/b)×100=% SPF remaining

(a) is SPF value after 80 minutes of water exposure and (b) is initial SPF value.

### Example 16

### Sunscreen Composition Testing for Water-Resistant Properties

The base sunscreen composition of Instant Example 14 is formulated with the terpolymer of Instant Example 13 and compared with other commercially available polymers and copolymers. The composition of Instant Example 14 is prepared individually with the specified amount of each test polymer or copolymer. Commercially available polymers were added to the oil phase or water phase of the formulation, or post-added according to the recommendations described in the manufacturer's literature.

Each sunscreen formulation is evaluated according to the protocol of Instant Example 15. The experimental results are given below.

| Polymer Tested | wt/wt %* | % SPF remaining |
|---|---|---|
| None | 0 | 3.1 |
| Cosmedia DC | 3 | 4.5 |
| Polycrylene | 3 | 5.7 |
| DC FA 4001 CM | 3 | 6.0 |
| Silicone Acrylate | | |
| Ganex V-220 | 3 | 6.8 |
| DC FA 4002 ID | 3 | 7.4 |
| Silicone Acrylate | | |
| Phospholipon 90H | 3 | 35.0 |
| Dermacryl AQF | 3 | 40.5 |
| Ganex WP-660 | 3 | 53.0 |
| Stantiv OMA-2 | 3 | 53.3 |
| Dermacryl-79 | 3 | 55.9 |
| Allianz OPT | 3 | 81.1 |
| Avalure UR 450 | 3 | 83.5 |
| Instant Example 13 | 1 | 87.4 |

*The commercially available polymers are added to the sunscreen composition at a 3% weight/weight of component (as active) based on the weight of the total composition.

Instant Example 13 is added at a 1% weight/weight of component (as active) based on the weight of the total composition. The terpolymer of example 13 is post added after the emulsion is formed.

Cosmedia DC [INCI: Hydrogenated Dimer Dilinoleyl/Dimethylcarbonate Copolymer] is a hydrogenated dimer Dilinoleyl/Dimethylcarbonate Copolymer and is obtained from Cognis.

Polycrylene [INCI: Polyester-8] which is a copolymer of adipic acid (q.v.) and neopentyl glycol (q.v.) end-capped with either octyldodecanol (q.v.) or a cyanodiphenylpropenoyl group and is obtained from RTD Hall Star.

DC FA 4001 CM Silicone Acrylate or Dow Corning FA 4001 CM Silicone Acrylate [INCI: Cyclopentasiloxane (and) Acrylates/Acrylates/Polytrimethylsiloxymethacrylate Copolymer] is a copolymer of polytrimethylsiloxymethacrylate and one or more monomers consisting of acrylic acid, methacrylic acid, or one of their simple esters dissolved in cyclopentasiloxane and is obtained from Dow Corning.

Ganex V-220 [INCI: VP/Eicosene Copolymer] is a copolymer of vinylpyrrolidone and eicosene and is obtained from ISP.

DC FA 4002 ID Silicone Acrylate or Dow Corning FA 4002 ID Silicone Acrylate [INCI: Isododecane (and) Acrylates/Acrylates/Polytrimethylsiloxymethacrylate Copolymer] is a copolymer of polytrimethylsiloxymethacrylate and one or more monomers consisting of acrylic acid, methacrylic acid, or one of their simple esters dissolved in isododecane and is obtained from Dow Corning.

Phospholipon 90H [INCI: Hydrogenated Lecithin] is hydrogenated lecithin and is obtained from Phospholipid GmbH.

Dermacryl AQF [INCI: Acrylates Copolymer] is a copolymer of acrylates and is obtained from National Starch and Chemical Company.

Ganex WP-660 [INCI: Triacontanyl PVP] is a copolymer of vinyl pyrrolidone and 1-triacontane and is obtained from ISP.

Stantiv OMA-2 [INCI: Octadecene/MA Copolymer (and) Methyl Acetyl Ricinoleate (and) Di-Methylheptyl Adipate] is a linear copolymer of maleic anhydride and octadecene and is dissolved a mixture of methyl acetyl ricinoleate and dimethylheptyl adipate.

Dermacryl-79 [INCI: Acrylates/Octylacrylamide Copolymer] is a copolymer of octylacrylamide and one or more monomers consisting of acrylic acid, methacrylic acid or one of their simple esters and is obtained from National Starch and Chemical Company.

Allianz OPT [INCI: Acrylates/C12-22 Alkyl Methacrylate Copolymer] is a copolymer of: methacrylic acid, methyl methacrylate, butyl acrylate, and cetyl-eicosinyl methacrylate and is obtained from ISP.

Avalure UR 450 [INCI: PPG-17/IPDI/DMPA Copolymer] is a copolymer of PPG-17, isophorone diisocyanate and dimethylol propionic acid monomers and is obtained from Noveon.

The data demonstrate the instant terpolymer provide excellent water proofing properties in sunscreen compositions at one-third of the concentration when compared to other polymers and copolymers of the prior art and commerce.

### Example 17

### Sunscreen Composition Testing for Water-Resistant Properties

A commercial sunscreen formulation (Cetaphil SPF 15, Galderma) is obtained and is thoroughly mixed individually with the specified amount of each test polymer or copolymer. Each sunscreen formulation is evaluated according to the protocol of Instant Example 15. The experimental results are given below.

| Polymer Tested | wt/wt %* | % SPF remaining |
|---|---|---|
| None | 0 | 10.1 |
| Dermacryl AQF | 2 | 9.1 |
| Allianz OPT | 1 | 49.1 |
| Instant Example 13 | 1 | 62.9 |

*The polymers are added to the sunscreen composition on a % weight/weight of component (as active) based on the weight of the total composition.

Dermacryl AQF is a copolymer of acrylates and is obtained from National Starch and Chemical Company.

Allianz OPT is a copolymer of: methacrylic acid, methyl methacrylate, butyl acrylate, and cetyl-eicosinyl methacrylate and is obtained from ISP.

Cetaphil SPF 15 is a commercial sinscreen formulation that contains sunscreen actives: Avobenzone 3%; Octocrylene 10%; and

### Inactive Ingredients (Function):

Water (solvent),
Isopropyl adipate (emollient, solvent),
Cyclomethicone (emollient, solvent),
Glyceryl Stearate (and) PEG-100 Stearate (emulsifier, non-ionic),
Glycerin (humectant),
Polymethyl Metacrylate (spherical particulate to improve the skin feel,
Phenoxyethanol (preservative),
Benzyl Alcohol (preservative),
Acrylates/C10-30 Alkyl Acrylate Crosspolymer (polymeric emulsifier, rheology modifier),
Tocopheryl Acetate (antioxidant),
Carbomer (rheology modifier),
Disodium EDTA (chelating agent), and
Triethanolamine (pH adjustor).

The data demonstrate the instant terpolymer provide excellent water proofing properties in sunscreen compositions when compared to other polymers and copolymers of the prior art and commerce.

### Example 18

### Very Water-Resistant Properties of the Sunscreen Formulations

The water-resistant properties of the instant terpolymers are studied according to: the FDA Final Monograph “Evaluation of Sunscreen Efficacy—Sun Protection Factor (SPF) Assay and Very Water-resistant Assay” (in vivo). The instant terpolymers are studied at 1% w/w based on solids and the data obtained from the in vivo evaluation of the very water-resistant properties of the sunscreen formulations containing the instant terpolymers is given below.

| | | Final SPF | PerCent SPF |
|---|---|---|---|
| Sample | Initial SPF | After Exposure | After Exposure |
| Control* | 15.26 | 7.46 | 48 |
| Instant 13 | 15.0 | 11.14 | 74 |

*A commercial daily moisturizing sunscreen formulation (Cetaphil SPF 15, Lot 049957, Galderma) is obtained and is thoroughly mixed with the specified amount of each instant terpolymer.

It is found that, at concentration 1% w/w based on solids, the instant terpolymers provide a significant improvement of very water-resistant properties of a sunscreen formulation.

### Example 19

### Residual Monomer Level

The Instant Terpolymer of Example 13 is analyzed for the residual monomer vinyl imidazole and is found to contain 240 ppm by quantitative gas chromatographic analysis. Instant Example 13 is subjected to boiling water stripping (distillation) three different times. The amount of vinyl imidazole is determined to be 180 ppm, 154 ppm, and 114 ppm, respectively.

Gel Permeation Chromoatographic (GPC) analysis is performed for all samples after steam distillation. The purified samples have identical GPC spectra as compared to the original unpurified terpolymer sample, therefore the polymer backbone remains unchanged after purification.

### Example 20

### Residual Monomer Level

The purification procedure of Instant Example 19 is repeated. A residual monomer level of 50 ppm is obtained.

### Example 20a

### Residual Monomer Level

The purification procedure of Instant Example 19 is repeated. A residual monomer level of 5 ppm is obtained.

### Example 21

### Contact Angles of Water and Surface Properties

A test methodology that utilizes measurements of the contact angle of water to quantify the effects on the surface properties of a skin-substitute substrate is employed. This methodology is used as an effective tool for optimizing product development, differentiating among skin care products, competitive benchmarking, and screening of the polymers. It is described in the article entitled “Correlating Water Contact Angles and Moisturization/Sensory Claims” by Olga V. Dueva-Koganov, Scott Jaynes, Colleen Rocafort, Shaun Barker and Jianwen Mao—Cosmetics & Toiletries, January 2007, Vol. 122, No. 1, pp. 20-27. The data presented in the graph of this article shows that contact angle measurements can be used to quantify and compare the effects of skin care products on the surface properties of a skin-like substrate and is presented in tabular form below. Products that generate relatively low contact angles tend to make more sensory claims related to light and non-greasy feel, while products that produce relatively high contact angles tend to make more claims related to long-term moisturization.

| | % Products | % Products | % Products |
|---|---|---|---|
| Contact Angles | in A* | in B** | in C*** |
| 40-50 | 100 | 50 | 0 |
| 50-60 | 100 | 40 | 20 |
| 60-70 | 60 | 30 | 60 |
| 70-80 | 70 | 15 | 70 |
| 80-90 | 50 | 0 | 100 |

*A = Products that are light and/or nongreasy.
**B = Products that provide 8-12 hours moisturization.
***C = Products that provide 24 hours moisturization.

### Example 22

### Measurement of Contact Angles after Application of the Instant Terpolymers

Contact angles are measured instrumentally according to the static or sessile drop method and using deionized water as a probe solution and VITRO SKIN that mimics the surface properties of human skin as a substrate. A piece of hydrated substrate is mounted in a glassless slide and air-dried in a flat position with application side up for 15 minutes. It is used as a reference for untreated substrate during the contact angle measurements. Exactly 0.032 g of aqueous solutions or dispersions of test polymers are applied evenly across a 4 cm×4 cm section of the substrate (on the “skin topography” side). Immediately after product application, the product is rubbed into the substrate with a finger covered with fingercot. After that the substrate is placed in a slide mount and air-dried for 15 minutes. Before measurements, substrate is removed from the slide mount and cut to several small pieces, which are used for the measurements. The use of small size piece is necessary to assure its flat position on the sample table. Extra care is taken to ensure that the rough side is up and the film is flat. Contact angle measurements are conducted expeditiously—within approximately 1 minute. Controlled humidity conditions are utilized.

### Materials

DSA-10 Contact Angle Measuring System, Krüss Gmb.

VITRO SKIN (N-19), IMS Inc.,

Powder Free Rubber Finger Cots (# 11-392-9B) are available from the Fisher Scientific Catalog.

Instant terpolymers and competitive water-resistant polymers Allianz OPT (ISP) and Dermacryl AQF (National Starch) are evaluated according to the methodology described above.

Contact Angles after Application of the Polymers

| Polymer Tested | wt/wt %* | Contact Angle, Theta (M) deg. |
|---|---|---|
| Control (Vitro Skin) | None | 103.6 |
| Instant Example 13 | 5 | 60.8 |
| Instant Example 13 | 2.5 | 62.8 |
| Instant Example 13 | 1 | 62.7 |
| Instant Example 13 | 0.5 | 60.3 |
| Instant Example 13 | 0.25 | 71.3 |
| Allianz OPT | 5 | 109.8 |
| Allianz OPT | 2.5 | 108.6 |
| Allianz OPT | 1 | 111.9 |
| Allianz OPT | 0.5 | 99.7 |
| Allianz OPT | 0.25 | 104.8 |
| Dermacryl AQF | 5 | 116.1 |
| Dermacryl AQF | 2.5 | 117.3 |

*Indicates aqueous solutions of the test sample as percent polymer solids based on the total weight of the solution.

The instant terpolymers and competitive water-resistant polymers demonstrate strong differences in their effects on the surface properties of VITRO SKIN. The results presented in the table above indicate that the instant terpolymers can potentially contribute to light skin feel a desirable characteristic for water-resistant polymers. On the contrary—the competitive benchmarks (Allianz OPT and Dermacryl AQF) generate primarily a hydrophobic modification of the substrate and are less likely to produce light skin feel.

### Example 23

### Sensory Characteristics of Formulations Containing Instant Terpolymers

Formulations of Instant Example 14 are prepared and tested for sensory characteristics according to testing protocols published in: 1) ASTM, American Society for Testing and Materials; Annual Book of ASTM Standards, E 1490-92 (reapproved 1997), or 2) Meilgaard M, Civille G, Carr B (2007), Sensory Evaluation Techniques, CRC Press, 4th ed.].

The results are given below.

| Sensory Characteristics | Form. A | Form. B | Form. C |
|---|---|---|---|
| Spreadability | 2 | 2 | 2 |
| Playtime | 3 | 3 | 3 |
| Softness | 4 | 4 | 4 |
| Shiny | 3 | 3 | 3 |
| Greasy | 2 | 2 | 2 |
| Sticky | 2 | 2 | 2 |

Form. A is Instant Example 14 with no instant terpolymers added.
Form. B is Instant Example 14 with 1 percent by weight (solids) of Example 13.
Form. C is Instant Example 14 with 2 percent by weight (solids) of Example 13.

These data demonstrate that the terpolymers of the instant invention do not negatively impact the sensory parameters of the formulation.

### Example 24

### Alcoholic Sunscreen Spray Compositions

| Ingredients | % WT. | Trade Name | Manufacturer |
|---|---|---|---|
| USP Ethanol | qs to | Ethyl Alcohol | Pharmaco/Aaper |
| 200 proof | 100.00 | | |
| C12-15 Alkyl | 4.00 | Finsolv TN | Finetex |
| Benzoate | | | |
| Homosalate | 8.00 | Parsol HMS | DSM |
| Octisalate | 5.00 | Escalol 587 | ISP |
| Octocrylene | 2.35 | Neo-Heliopan 303 | H&R |
| Avobenzone | 3.00 | Parsol 1789 | DSM |
| Oxybenzone | 6.00 | Escalol 567 | ISP |
| Example 13 | 0.00-0.25 | | Ciba |
| Terpolymer | | | |
| (as supplied) | | | |

Manufacturing Procedure: Combine all ingredients at RT, mix until uniform.

Very Water-resistant (80 min of water exposure), In Vitro

% SPF Remaining

| Random Terpolymer, % | Average | StDev |
|---|---|---|
| 0.00000 (Base) | 66.02 | 13.173 |
| 0.25000 | 4.64 | 0.495 |
| 0.12500 | 5.45 | 0.183 |
| 0.10000 | 4.73 | 0.884 |
| 0.06250 | 16.83 | 6.102 |
| 0.03125 | 42.51 | 19.450 |
| 0.01563 | 76.765 | 7.867 |
| 0.00781 | 106.950 | 4.801 |

Conclusions: in anhydrous alcoholic sunscreen sprays the random terpolymer improves in vitro water-resistant properties of the formulation at concentrations lower than 0.03%. Same testing protocol as example 15 above.

### Example 25

### Sunscreen Gel Creams Composition

| Trade Name | INCI-Name/Chemical-Name | Supplier | % w/w |
|---|---|---|---|
| Part A | | | |
| Water | Water | | q.s.p 100% |
| Dissolvine NA-2-P | Disodium EDTA | Akzo Nobel | 0.20 |
| TINODERM ®SG-P | Aqua (and) Pentylene Glycol | Ciba | 2.00 |
| | (and) Sclerotium Gum | | |
| Terpolymer of | | | 1.00 |
| Example 13¹ | | | |
| Part B | | | |
| Eumulgin B 1 | Ceteareth-12 | Cognis | 1.00 |
| Cetiol B | Dibutyl Adipate | Cognis | 5.00 |
| Dermofeel TC-7 | Triheptanoin | Dr. Straetmans | 3.00 |
| Uvinul A Plus | Diethylamino Hydroxybenzoyl | BASF | 10.00 |
| | Hexyl Benzoate | | |
| Neo Heliopan E 1000 | Isoamyl p-Methoxycinnamate | Symrise | 7.50 |
| Uvinul T 150 | Ethylhexyl Triazone | BASF | 3.20 |
| TINOSORB ® S | Bis-Ethylhexyloxyphenol | Ciba | 1.50 |
| | Methoxyphenyl Triazine | | |
| TINOSORB ® M | Methylene Bis-Benzotriazoly | Ciba | 1.50 |
| | Tetramethylbutylphenol | | |
| | Tris-Biphenyl Triazine | Ciba | 2.00 |
| | Methanone, 1,1′-(1,4-piperazinediyl) | | 1.00 |
| | bis[1-[2-[4-(diethylamino)-2-hydroxybenzoyl]phenyl]- | | |
| Part C | | | |
| TINOVIS ® ADE | Sodium Acrylates Copolymer (and) | Ciba | 1.20 |
| | Hydrogenated Polydecene (and) | | |
| | PPG-1 Trideceth-6 | | |
| Part D | | | |
| Dow Corning 246 | Cyclohexasiloxane (and) | Dow Corning | 4.00 |
| Fluid | Cyclopentasiloxane | | |
| Part E | | | |
| Phenonip | Phenoxyethanol | Clariant | 1.00 |
| | (and) Methylparaben (and) | | |
| | Ethylparaben (and) Butylparaben | | |
| | (and) Propylparaben (and) | | |
| | Isobutylparaben | | |
| TINODERM ® E | Aqua (and) Tocopheryl Acetate | Ciba | 0.50 |
| | (and)Caprylic/Capric Triglyceride | | |
| | (and) Polysorbate 80 (and) Lecithin | | |

Manufacturing Instruction
Heat part A and B at 75° C.
Under Ultra Turrax mixer, add part B (75° C.) into part A (75° C.).
Add immediately part C, mix until homogeneous.
By 60° C., add part D and mix until homogeneous.
At room temperature, add part E and mix until homogeneous.
¹The terpolymer of the invention may be added in the water phase or post added after generation of the emulsion.

### Example 26

### Water-Resistance Testing for Terpolymer of Example 13

| | No terpolymer | 1% terpolymer | 2% terpolymer |
|---|---|---|---|
| Ingredient | added | from example 13 | from example 13 |
| Water | 57.75 | 55.75 | 53.75 |
| Disodium EDTA | 0.20 | 0.20 | 0.20 |
| Glycerine | 3.00 | 3.00 | 3.00 |
| Xanthan Gum | 0.22 | 0.22 | 0.22 |
| Bis-Ethylhexyloxyphenol | 1.50 | 1.50 | 1.50 |
| Methoxyphenyl Triazine | | | |
| Ethylhexyl | 7.50 | 7.50 | 7.50 |
| Methoxycinnamate | | | |
| Butyl | 3.00 | 3.00 | 3.00 |
| Methoxydibenzoylmethane | | | |
| Ethylhexyl Salicylate | 2.00 | 2.00 | 2.00 |
| Glyceryl Stearate and | 3.50 | 3.50 | 3.50 |
| PEG-100 Stearate | | | |
| Cetyl Alcohol | 1.00 | 1.00 | 1.00 |
| Dibutyl adipate | 5.00 | 5.00 | 5.00 |
| Dicaprylyl Carbonate | 5.00 | 5.00 | 5.00 |
| Diethylhexyl Carbonate | 2.00 | 2.00 | 2.00 |
| Sodium Acrylates | 0.83 | 0.83 | 0.83 |
| Copolmer and Mineral Oil | | | |
| and PPG-a Trideceth-6 | | | |
| Cyclopentasiloxane | 2.00 | 2.00 | 2.00 |
| Nylon-12 | 1.50 | 1.50 | 1.50 |
| Methylene Bis- | 3.00 | 3.00 | 3.00 |
| Benzotriazolyl | | | |
| Tetramethylbutylphenol | | | |
| Phenoxyethanol and | 1.00 | 1.00 | 1.00 |
| Methylparaben and | | | |
| Ethylparaben and | | | |
| Butylparaben and | | | |
| Propylparaben and | | | |
| Isobutylparaben | | | |
| Terpolymer of example 13 | 0.00 | 2.00 | 4.00 |
| (50% active) | | | |
| Total | 100.00 | 100.00 | 100.00 |
| pH | 5.31 | 5.57 | 5.69 |
| Viscosity | RV5/10: | RV5/10: | RV5/10: |
| | 12680 MPa/s | 219600 mPa/s | 2152-0 mPa/s |
| | −31.7% | −49% | −53.8% |

### Parameters for the In Vitro Efficacy of Sunscreen for the Formulation Example 26

| Test parameter | Water-resistant Testing Conditions in Vitro | |
|---|---|---|
| Substrate | Vitro Skin ™ | |
| Application dose | 2 | mg/cm² |
| Number of repetitions | 3 | |
| Water exposure | 80 | min. |
| Quantity of water | 17 | liters |
| Water bath Temperature | 30-31 | C. |
| Mixing | 300 | rpm |

| | |
|---|---|
| Sample drying | RT |

### Comparative Water-resistance Results Using Formulations of Example 26.

| Sample | Trial 1* | Trial 2 | Trial 3 | Av +/− St Dev |
|---|---|---|---|---|
| No Polymer | 31 | 36 | 37 | 35% +/− 4% |
| added | | | | |
| 1% terpolymer | 72 | 78 | 77 | 76% +/− 4% |
| from example | | | | |
| 13 | | | | |
| 2% terpolymer | 83 | 88 | 96 | 89% +/− 7% |
| from example | | | | |
| 13 | | | | |

*The numbers respresent calculated SPF after water rinsing as in the testing protocol carried out in example 15 above. Correct, Olga?

### Example 27

### This Application Example shows the Improved SPF Achieved Upon Addition of the Random Terpolymer of Example 13 to a Preformulated Sunscreen Cream

| | | | SPF after | Stdev | UVA/UVB | UVA/UVAB | |
|---|---|---|---|---|---|---|---|
| | SPF | StDev | water | after | ratio | ratio after | % SPF |
| Sample | initial | Initial | rinsing | rinsing | initial | rinsing | remaining |
| Neutrogena ¹ | 16.96 | 1.33 | 12.98 | 1.06 | 0.444 | 0.375 | 76.533 |
| with 1% | 21.77 | 0.59 | 17.88 | 1.40 | 0.476 | 0.411 | 82.131 |
| terpolymer | | | | | | | |
| from | | | | | | | |
| example 13 | | | | | | | |
| (active) | | | | | | | |
| Neutogena | 17.61 | 1.60 | 10.64 | 0.86 | 0.427 | 0.319 | 60.420 |
| with 2% | 16.82 | 0.37 | 9.92 | 0.70 | 0.418 | 0.317 | 58.977 |
| terpolymer | | | | | | | |
| from | | | | | | | |
| example 13 | | | | | | | |
| (active) | | | | | | | |
| Neutogena | 18.46 | 1.36 | 7.76 | 1.36 | 0.452 | 0.391 | 42.037 |
| Base only | 15.70 | 0.64 | 6.08 | 0.44 | 0.441 | 0.369 | 38.726 |

¹ Neutrogena Healthy Skin SPF-15
Protocol of example 15 is used.

The improved resulting average SPF % efficiency in the preformed formulations achieved by addition of the terpolymer of example 13 to the commercial formulation is clear.

| Sample | Average % Efficiency | StDev |
|---|---|---|
| Neutrogena ¹ with 1% | 79.33 | 3.96 |
| terpolymer from example | | |
| 13 (active) | | |
| Neutogena with 2% | 59.70 | 1.02 |
| terpolymer from example | | |
| 13 (active) | | |
| Neutogena Base only | 40.38 | 2.34 |

### Example 28

### Daily Protection Care

Smooth application, non greasy skin feel

| Composition | INCI-Name/Chemical- | | | | | |
|---|---|---|---|---|---|---|
| Trade Name | Name | Supplier | % w/w (as supplied) | | | |
| Part A | | | | | | |
| Arlacel 165 V | Glyceryl Stearate (and) | Uniqema | 1.80 | 1.80 | 1.80 | 1.80 |
| | PEG-100 Stearate | | | | | |
| Tegosoft TN | C12-15 Alkyl Benzoate | Degussa | 7.00 | 7.00 | 7.00 | 7.00 |
| Dermofeel BGC | Butylene Glycol | Dr. | 5.00 | 5.00 | 5.00 | 5.00 |
| | Dicaprylate/Dicaprate | Straetmans | | | | |
| Tegosoft P | Isopropyl Palmitate | Degussa | 2.00 | 2.00 | 2.00 | 2.00 |
| Lanette O | Cetearyl Alcohol | Cognis | 1.50 | 1.50 | 1.50 | 1.50 |
| Neo Heliopan 303 | Octocrylene | Symrise | 5.00 | 5.00 | 5.00 | 5.00 |
| Parsol 1789 | Butyl | DSM | 2.50 | 2.50 | 2.50 | 2.50 |
| | Methoxydibenzoylmethane | | | | | |
| TINOSORB ® S | Bis-Ethylhexyloxyphenol | Ciba | 1.70 | 1.70 | 1.70 | 1.70 |
| | Methoxyphenyl Triazine | | | | | |
| Part B | | | | | | |
| Water | Water | | qs to 100% | qs to 100% | qs to 100% | qs to 100% |
| Propylene Glycol | Propylene Glycol | Rita | 5.00 | 5.00 | 5.00 | 5.00 |
| | | Corporation | | | | |
| | Random Terpolymer | Ciba | 0.50 | 1.00 | 2.00 | 4.00 |
| | Example 13** | | | | | |
| Dissolvine NA-2-P | Disodium EDTA | Akzo Nobel | 0.20 | 0.20 | 0.20 | 0.20 |
| Amphisol K | Potassium Cetyl Phosphate | DSM | 2.50 | 2.50 | 2.50 | 2.50 |
| Part C | | | | | | |
| TINOCARE ®GL | Sclerotium Gum | Ciba | 3.00 | 3.00 | 3.00 | 3.00 |
| Part D | | | | | | |
| Eusolex 232 | Phenylbenzimidazole | Merck | 1.50 | 1.50 | 1.50 | 1.50 |
| | Sulfonic Acid | | | | | |
| Water | Water | | 3.75 | 3.75 | 3.75 | 3.75 |
| Tris Amino | Tromethamine | Angus | 0.66 | 0.66 | 0.66 | 0.66 |
| Part E | | | | | | |
| SALCARE ® SC91 | Sodium Acrylates | Ciba | 1.00 | 1.00 | 1.00 | 1.00 |
| | Copolymer (and) Mineral | | | | | |
| | Oil (and) PPG-1 | | | | | |
| | Trideceth-6 | | | | | |
| Part F | | | | | | |
| Dow Corning | Cyclomethicone | Dow | 3.00 | 3.00 | 3.00 | 3.00 |
| 345 Fluid | | Corning | | | | |
| Preservative | | | qs | | | |
| TINODERM ® A | Aqua (and) Retinyl | Ciba | 1.00 | 1.00 | 1.00 | 1.00 |
| | Palmitate (and) | | | | | |
| | Caprylic/Capric | | | | | |
| | Triglyceride (and) | | | | | |
| | Polysorbate 80 (and) | | | | | |
| | Lecithin (and) | | | | | |
| | Benzalkonium Chloride | | | | | |
| TINODERM ® E | Aqua (and) Tocopheryl | Ciba | 1.00 | 1.00 | 1.00 | 1.00 |
| | Acetate (and) | | | | | |
| | Caprylic/Capric | | | | | |
| | Triglyceride (and) | | | | | |
| | Polysorbate 80 (and) | | | | | |
| | Lecithin | | | | | |

Manufacturing Instruction
Heat up part A and part B (without Amphisol K) to 80° C., then add Amphisol K into part B and stir slowly during few minutes. Add part A into part B, homogenize with an Ultra Turrax. Add part C. Cool down under continuous stirring and add part D by around 60° C. Afterwards add part E by around 45° C., then part F below 40° C. Alternatively, Random Terpolymer can be post-added to the system after emulsion is formed.
**Addition of Random Terpolymer improves water-resistant properties of the formulation.

### Example 29

### Soft Suncare Cream

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Emulium 22 | Tribehenin PEG-20 Esters | Gattefosse | 5.00 | 5.00 | 5.00 | 5.00 |
| Cetiol B | Dibutyl Adipate | Cognis | 5.00 | 5.00 | 5.00 | 5.00 |
| Crodamol PMP | PPG-2 Myristyl Ether | Croda | 5.00 | 5.00 | 5.00 | 5.00 |
| | Propionate | | | | | |
| Neo Heliopan 303 | Octocrylene | Symrise | 6.00 | 6.00 | 6.00 | 6.00 |
| Parsol 1789 | Butyl | DSM | 2.50 | 2.50 | 2.50 | 2.50 |
| | Methoxydibenzoylmethane | | | | | |
| TINOSORB ® S | Bis-Ethylhexyloxyphenol | Ciba | 2.00 | 2.00 | 2.00 | 2.00 |
| | Methoxyphenyl Triazine | | | | | |
| Part B | | | | | | |
| Water | Water | qs to | 100% | 100% | 100% | 100% |
| Dissolvine NA-2-P | Disodium EDTA | Akzo Nobel | 0.20 | 0.20 | 0.20 | 0.20 |
| | Random Terpolymer | Ciba | 2.00 | 2.00 | 2.00 | 2.00 |
| | Example 13 ** | | | | | |
| TINOCARE ®GL | Sclerotium Gum | Ciba | 2.00 | 2.00 | 2.00 | 2.00 |
| Keltrol T | Xanthan Gum | Rahn | 0.50 | 0.50 | 0.50 | 0.50 |
| Part C | | | | | | |
| Dow Corning | Cyclohexasiloxane (and) | Dow | 4.00 | 4.00 | 4.00 | 4.00 |
| 246 Fluid | Cyclopentasiloxane | Corning | | | | |
| Dow Corning | Dimethicone/Vinyl | Dow | 3.00 | 3.00 | 3.00 | 3.00 |
| 9701 | dimethicone crosspolymer | Corning | | | | |
| | (and) Cosmetic Powder | | | | | |
| | silica | | | | | |
| Part D | | | | | | |
| Water | Water | | 5.00 | 5.00 | 5.00 | 5.00 |
| Eusolex 232 | Phenylbenzimidazole | Merck | 2.00 | 2.00 | 2.00 | 2.00 |
| | Sulfonic Acid | | | | | |
| Tris Amino | Ultra Pur Tromethamine | Angus | q.s. pH = 7 | q.s. pH = 7 | q.s. pH = 7 | q.s. pH = 7 |
| | | Chemie | | | | |
| Part E | | | | | | |
| TINOSORB ® M | Methylene Bis- | Ciba | 10.00 | 5.00 | 5.00 | 3.00 |
| | Benzotriazolyl | | | | | |
| | Tetramethylbutylphenol | | | | | |
| | (and) Aqua (and) Decyl | | | | | |
| | Glucoside (and) Propylene | | | | | |
| | Glycol (and) Xanthan Gum | | | | | |
| | Tris-Biphenyl Triazine, | Ciba | | 3.00 | | 3.00 |
| | 50% micronized active in | | | | | |
| | aqueous solution | | | | | |
| | Methanone, 1,1′-(1,4- | Ciba | | | 1.00 | 2.00 |
| | piperazinediyl) bis[1-[2- | | | | | |
| | [4-(diethylamino)-2- | | | | | |
| | hydroxybenzoyl]phenyl]- | | | | | |
| | (CAS No: 919803-06-8), | | | | | |
| | 50% micronized active in | | | | | |
| | aqueous solution | | | | | |
| Part F | | | | | | |
| TINODERM ®E | Aqua (and) Tocopheryl | Ciba | 0.50 | 0.50 | 0.50 | 0.50 |
| | Acetate (and) | | | | | |
| | Caprylic/Capric | | | | | |
| | Triglyceride (and) | | | | | |
| | Polysorbate 80 (and) | | | | | |
| | Lecithin | | | | | |
| | Preservative | qs | qs | qs | qs | qs |

Manufacturing Instruction
Heat part A at 75° C. until homogeneous. Heat part B without Keltrol T at 75° C. until homogeneous.
Add Keltrol T in part B under turrax and mix until homogeneous. Add part A at 75° C. into part B at 75° C. and mix until homogeneous, homogenize with an Ultra Turrax. At 60° C., add part C and mix until homogeneous.
Prepare part D at room temperature, then add part D into the emulsion by around 50° C. and mix until homogeneous. By
room temperature add part E and part F, mix until homogeneous. Alternatively, Random Terpolymer can be post-added
to the system after emulsion is formed.
** Addition of Random Terpolymer improves water-resistant properties of the formulation.

### Example 30

### Emulsifier Free Smooth Sun Gel

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Finsolv EB | Ethylhexyl Benzoate | Finetex | 5.00 | 5.00 | 5.00 | 5.00 |
| DUB VCI10 | Isodecyl Neopentanoate | Dubois | 3.00 | 3.00 | 3.00 | 3.00 |
| | Stearinerie | | | | | |
| Neo Heliopan 303 | Octocrylene | Symrise | 10.00 | 10.00 | 10.00 | 10.00 |
| Neo Heliopan, | Ethylhexyl Salicylate | Symrise | 5.00 | 5.00 | 5.00 | 5.00 |
| Type OS | | | | | | |
| Parsol 1789 | Butyl | DSM | 4.00 | 4.00 | 4.00 | 4.00 |
| | Methoxydibenzoylmethane | | | | | |
| Eusolex T-AVO | Titanium Dioxide (and) | Merck | 4.00 | 4.00 | 4.00 | 4.00 |
| | Silica | | | | | |
| TINOSORB ® S | Bis-Ethylhexyloxyphenol | Ciba | 2.00 | 2.00 | 2.00 | 2.00 |
| | Methoxyphenyl Triazine | | | | | |
| Part B | | | | | | |
| Water | Water | qs to 100% | qs to 100% | qs to 100% | qs to 100% | qs to 100% |
| Glycerin 85% | Glycerin | Fluka | 5.00 | 5.00 | 5.00 | 5.00 |
| | Random Terpolymer | Ciba | 0.50 | 1.50 | 2.50 | 3.50 |
| | Example 13 ** | | | | | |
| Keltrol RD | Xanthan Gum | Rahn | 0.20 | 0.20 | 0.20 | 0.20 |
| Dissolvine NA-2-P | Disodium EDTA | Akzo Nobel | 0.20 | 0.20 | 0.20 | 0.20 |
| TINODERM ®SG-P | Aqua (and) Pentylene | Ciba | 4.00 | 4.00 | 4.00 | 4.00 |
| | Glycol (and) Sclerotium | | | | | |
| | Gum | | | | | |
| TINOVIS ®GTC | Acrylates/Beheneth-25 | Ciba | 2.50 | 2.50 | 2.50 | 2.50 |
| | Methylacrylate Copolymer | | | | | |
| Part C | | | | | | |
| Sodium Hydroxide | Water (and) Sodium | Fluka | qs | qs | qs | qs |
| (30% solution) | Hydroxide | | | | | |
| Part D | | | | | | |
| TINODERM ® E | Aqua (and) Tocopheryl | Ciba | 3.00 | 3.00 | 3.00 | 3.00 |
| | Acetate (and) | | | | | |
| | Caprylic/Capric | | | | | |
| | Triglyceride (and) | | | | | |
| | Polysorbate 80 (and) | | | | | |
| | Lecithin | | | | | |
| DOW CORNING ® | Dimethicone (and) | Dow | 2.00 | 2.00 | 2.00 | 2.00 |
| 1503 FLUID | Dimethiconol | Corning | 2.00 | 2.00 | 2.00 | 2.00 |
| Orgasol 2002 D | Nylon-12 | Atofina | | | | |
| NAT COS 20 | | | | | | |
| Microns | | | | | | |
| | Preservative | | qs | qs | qs | qs |

Manufacturing Instruction
Mix the ingredients of part B, disperse Xanthan Gum
Heat up part A to melt the filters, when melted, add Titanium Dioxide and homogenize until homogeneous. Cool down to room temperature. Incorporate part A into part B with an Ultra Turrax type device. Neutralize with part C to pH > 6.
Finally add the ingredients of part D in the listed order. Alternatively, Random Terpolymer can be post-added to the system after gel is formed.
** Addition of Random Terpolymer improves the water-resistant properties of the formulation.

### Example 31

### Sun Protect Fluid

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Sensanov WR | C20-22 Alkyl Phosphate | Seppic | 4.00 | 4.00 | 4.00 | 4.00 |
| | (and) C20-C22 Alcohols | | | | | |
| Cetiol B | Dibutyl Adipate | Cognis | 5.00 | 5.00 | 5.00 | 5.00 |
| Neo Heliopan 303 | Octocrylene | Symrise | 10.00 | 10.00 | 10.00 | 10.00 |
| Parsol 1789 | Butyl | DSM | 3.00 | 3.00 | 3.00 | 3.00 |
| | Methoxydibenzoylmethane | | | | | |
| Uvasorb HEB | Diethylhexyl Butamino | 3VSigma | 2.00 | 2.00 | 2.00 | 2.00 |
| | Triazone | | | | | |
| TINOSORB ® S | Bis-Ethylhexyloxyphenol | Ciba | 2.00 | 2.00 | 2.00 | 2.00 |
| | Methoxyphenyl Triazine | | | | | |
| Parsol MCX | Ethylhexyl | DSM | 0.20 | 0.20 | 0.20 | 0.20 |
| | Methoxycinnamate | | | | | |
| Part B | | | | | | |
| Water | Water | | q.s. to 100% | q.s. to 100% | q.s. to 100% | q.s. to 100% |
| Dissolvine NA-2-P | Disodium EDTA | Akzo Nobel | 0.20 | 0.20 | 0.20 | 0.20 |
| | Random Terpolymer | Ciba | 1.00 | 1.00 | 1.00 | 1.00 |
| | Example 13** | | | | | |
| TINODERM ®SG-P | Pentylene Glycol (and) | Ciba | 2.00 | 2.00 | 2.00 | 2.00 |
| | Sclerotium Gum | | | | | |
| Tris Amino | Ultra Pur Tromethamine | Angus | 0.50 | 0.50 | 0.50 | 0.50 |
| | | Chemie | | | | |
| Part C | | | | | | |
| Dow Corning 246 | Cyclohexasiloxane (and) | Dow | 4.00 | 4.00 | 4.00 | 4.00 |
| | | Corning | | | | |
| Fluid | Cyclopentasiloxane | | | | | |
| Dry- Flo Pure | Aluminum Starch | National | 4.00 | 4.00 | 4.00 | 4.00 |
| | Octenylsuccinate | Starch & | | | | |
| | | Chemical | | | | |
| | | Company | | | | |
| Part D | | | | | | |
| Water | Water | | 6.00 | 6.00 | 6.00 | 6.00 |
| Eusolex 232 | Phenylbenzimidazole | Merck | 1.00 | 1.00 | 1.00 | 1.00 |
| | Sulfonic Acid | | | | | |
| Tris Amino | Ultra Pur Tromethamine | Angus | 0.65 | 0.65 | 0.65 | 0.65 |
| | | Chemie | | | | |
| Part E | | | | | | |
| TINOSORB ® M | Methylene Bis- | Ciba | 12.00 | | 8.00 | |
| | Benzotriazolyl | | | | | |
| | Tetramethylbutylphenol | | | | | |
| | (and) Aqua (and) Decyl | | | | | |
| | Glucoside (and) Propylene | | | | | |
| | Glycol (and) Xanthan Gum | | | | | |
| TINOSORB ® M-PGL | Methylene Bis- | Ciba | | 12.00 | | 8.00 |
| | Benzotriazolyl | | | | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | 0-2.00 | 2.00 | 2.00 |
| | 50% micronized active in | | | | | |
| | aqueous solution | | | | | |
| | Methanone, 1,1′-(1,4- | Ciba | | 0-1.00 | 1.00 | 1.00 |
| | piperazinediyl) bis[1-[2-[4- | | | | | |
| | (diethylamino)-2- | | | | | |
| | hydroxybenzoyl]phenyl]- | | | | | |
| | (CAS No: 919803-06-8), | | | | | |
| | 50% micronized active in | | | | | |
| | aqueous solution | | | | | |
| Part F | | | | | | |
| Phenonip | Phenoxyethanol (and) | Clariant | 1.00 | 1.00 | 1.00 | 1.00 |
| | Methylparaben (and) | | | | | |
| | Ethylparaben (and) | | | | | |
| | Butylparaben (and) | | | | | |
| | Propylparaben (and) | | | | | |
| | Isobutylparaben | | | | | |
| TINODERM ® E | Aqua (and) Tocopheryl | Ciba | 0.50 | 0.50 | 0.50 | 0.50 |
| | Acetate (and) | | | | | |
| | Caprylic/Capric | | | | | |
| | Triglyceride (and) | | | | | |
| | Polysorbate 80 (and) | | | | | |
| | Lecithin | | | | | |

Manufacturing Instruction
Heat part A and B to 75° C. Add A (75° C.) into part B (75° C.) and homogenize.
Cool down. At 60° C., add part C and mix until homogeneous. Then add part D, mix until homogeneous. At room temperature, add part E and mix until homogeneous.
Finally, add part F, mix until homogeneous. Alternatively, Random Terpolymer can be post-added to the system after system is formed.
**Addition of Random Terpolymer improves water-resistant properties of the formulation.

### Example 32

### Advanced UV Protection Sun Cream

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Apifil | PEG-8 Beeswax | Gattefossé | 5.00 | 5.00 | 5.00 | 5.00 |
| Emulcire ®61 | Cetyl Alcohol (and) | Gattefossé | 4.00 | 4.00 | 4.00 | 4.00 |
| WL2659 | Ceteth-20 (and) Steareth- | | | | | |
| | 20 | | | | | |
| Cetiol B | Dibutyl Adipate | Cognis | 5.00 | 5.00 | 5.00 | 5.00 |
| Neo Heliopan 303 | Octocrylene | Symrise | 10.00 | 10.00 | 10.00 | 10.00 |
| Neo Heliopan, | Ethylhexyl Salicylate | Symrise | 4.00 | 4.00 | 4.00 | 4.00 |
| Type OS | | | | | | |
| Parsol 1789 | Butyl | DSM | 3.00 | 3.00 | 3.00 | 3.00 |
| | Methoxydibenzoylmethane | | | | | |
| TINOSORB ® S | Bis-Ethylhexyloxyphenol | Ciba | 2.00 | 2.00 | 2.00 | 2.00 |
| | Methoxyphenyl Triazine | | | | | |
| Uvasorb HEB | Diethylhexyl Butamino | 3VSigma | 1.00 | 1.00 | 1.00 | 1.00 |
| | Triazone | | | | | |
| Parsol MCX | Ethylhexyl | DSM | 0.50 | 0.50 | 0.50 | 0.50 |
| | Methoxycinnamate | | | | | |
| Part B | | | | | | |
| Water | Water | | q.s. to 100% | q.s. to 100% | q.s. to 100% | q.s. to 100% |
| Dissolvine NA-2-P | Disodium EDTA | Akzo Nobel | 0.20 | 0.20 | 0.20 | 0.20 |
| | Random Terpolymer | Ciba | 2.00 | 2.00 | 4.00 | 0.50 |
| | Example 13** | | | | | |
| TINODERM ®SG-P | Pentylene Glycol (and) | Ciba | 2.00 | 2.00 | 2.00 | 2.00 |
| | Sclerotium Gum | | | | | |
| Keltrol T | Xanthan Gum | Rahn | 0.30 | 0.30 | 0.30 | 0.30 |
| Part C | | | | | | |
| Dow Corning 246 | Cyclohexasiloxane (and) | Dow | 5.00 | 5.00 | 5.00 | 5.00 |
| Fluid | Cyclopentasiloxane | Corning | | | | |
| Part D | | | | | | |
| TINOSORB ® M | Methylene Bis- | Ciba | 12.00 | 12.00 | | |
| | Benzotriazolyl | | | | | |
| | Tetramethylbutylphenol | | | | | |
| | (and) Aqua (and) Decyl | | | | | |
| | Glucoside (and) Propylene | | | | | |
| | Glycol (and) Xanthan | | | | | |
| | Gum | | | | | |
| TINOSORB ® M-PGL | Methylene Bis- | Ciba | | | 12.00 | 4.00 |
| | Benzotriazolyl | | | | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | | | 4.00 |
| | 50% micronized active in | | | | | |
| | aqueous solution | | | | | |
| | Methanone, 1,1′-(1,4- | Ciba | | | | 1.00 |
| | piperazinediyl) bis[1-[2- | | | | | |
| | [4-(diethylamino)-2- | | | | | |
| | hydroxybenzoyl]phenyl]- | | | | | |
| | (CAS No: 919803-06-8), | | | | | |
| | 50% micronized active in | | | | | |
| | aqueous solution | | | | | |
| Part E | | | | | | |
| Phenonip | Phenoxyethanol (and) | Clariant | 1.00 | 1.00 | 1.00 | 1.00 |
| | Methylparaben (and) | | | | | |
| | Ethylparaben (and) | | | | | |
| | Butylparaben (and) | | | | | |
| | Propylparaben (and) | | | | | |
| | Isobutylparaben | | | | | |
| TINODERM ® E | Aqua (and) Tocopheryl | Ciba | 0.50 | 0.50 | 0.50 | 0.50 |
| | Acetate (and) | | | | | |
| | Caprylic/Capric | | | | | |
| | Triglyceride (and) | | | | | |
| | Polysorbate 80 (and) | | | | | |
| | Lecithin | | | | | |

**Addition of Random Terpolymer improves water-resistant properties of the formulation.
Manufacturing Instruction
Heat part A at 75° C. until homogeneous. Heat part B without Keltrol T at 75° C. At 75° C. under quick Ultra Turrax
mixer, add Keltrol T to part B and mix until homogeneous. Add part A to part B at 75° C. and homogenize. By 60° C.,
add part C and mix until homogeneous. At room temperature, add part D and mix until homogeneous. Finally add part
E and mix until homogeneous. Alternatively, Random Terpolymer can be post-added to the system after system is
formed.

### Example 33

### Economical Sun Lotion

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Stearic Acid | Stearic Acid | J. T. Baker | 3.00 | 3.00 | 3.00 | 3.00 |
| Tegin M | Glyceryl Stearate | Evonik | 1.00 | 1.00 | 1.00 | 1.00 |
| Estol 3609 | Triethylhexanoin | Croda | 7.00 | 7.00 | 7.00 | 7.00 |
| Elefac I-205 | Octyldodecyl | Bernel | 7.00 | 7.00 | 7.00 | 7.00 |
| | Neopentanoate | (Rovi) | | | | |
| Stantiv OMA-2 | Octadecene/MA | Caschem | 2.00 | 2.00 | 2.00 | 2.00 |
| | Copolymer (and) various | | | | | |
| | esters | | | | | |
| Neo Heliopan 303 | Octocrylene | Symrise | 10.00 | 10.00 | 10.00 | 10.00 |
| Neo Heliopan, | Ethylhexyl Salicylate | Symrise | 5.00 | 5.00 | 5.00 | 5.00 |
| Type OS | | | | | | |
| Parsol 1789 | Butyl | DSM | 3.00 | 3.00 | 3.00 | 3.00 |
| | Methoxydibenzoylmethane | | | | | |
| Part B | | | | | | |
| Water | Water | | qs to 100% | qs to 100% | qs to 100% | qs to 100% |
| Glycerin | 85% Glycerin | Fluka | 5.00 | 5.00 | 5.00 | 5.00 |
| TINODERM ®SG-L | Lactic Acid (and) | Ciba | 2.00 | 2.00 | 2.00 | 2.00 |
| | Sclerotium Gum (and) | | | | | |
| | Sodium Lactate | | | | | |
| Dissolvine NA-2-P | Disodium EDTA | Akzo | 0.20 | 0.20 | 0.20 | 0.20 |
| | | Nobel | | | | |
| | Random Terpolymer | Ciba | 2.00 | 3.00 | 2.50 | 1.50 |
| | Example 13** | | | | | |
| Part C | | | | | | |
| TEA 99% T | Triethanolamine | Dow | 0.97 | 0.97 | 0.97 | 0.97 |
| | | Chemical | | | | |
| Part D | | | | | | |
| Dow Corning | Dimethicone | Dow | 1.00 | 1.00 | 1.00 | 1.00 |
| 200/350 cs Fluid | | Corning | | | | |
| Part E | | | | | | |
| Mackaderm Asto- | Aluminium Starch | Mc Intyre | 5.00 | 5.00 | 5.00 | 5.00 |
| Dry | Octenylsuccinate | | | | | |
| TINODERM ® E | Aqua (and) Tocopheryl | Ciba | 3.00 | 3.00 | 3.00 | 3.00 |
| | Acetate (and) | | | | | |
| | Caprylic/Capric | | | | | |
| | Triglyceride (and) | | | | | |
| | Polysorbate 80 (and) | | | | | |
| | Lecithin | | | | | |
| TINOSORB ® M | Methylene Bis- | Ciba | 3.00 | | 3.00 | |
| | Benzotriazolyl | | | | | |
| | Tetramethylbutylphenol | | | | | |
| | (and) Aqua (and) Decyl | | | | | |
| | Glucoside (and) Propylene | | | | | |
| | Glycol (and) Xanthan | | | | | |
| | Gum | | | | | |
| TINOSORB ® M-PGL | Methylene Bis- | Ciba | | 3.00 | | |
| | Benzotriazolyl | | | | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | | | 3.00 |
| | 50% micronized active in | | | | | |
| | aqueous solution | | | | | |
| | Methanone, 1,1′-(1,4- | Ciba | | | 1.00 | 1.00 |
| | piperazinediyl) bis[1-[2- | | | | | |
| | [4-(diethylamino)-2- | | | | | |
| | hydroxybenzoyl]phenyl]- | | | | | |
| | (CAS No: 919803-06-8), | | | | | |
| | 50% micronized active in | | | | | |
| | aqueous solution | | | | | |
| TINOSAN ® SDC | Citric Acid (and) Silver | Ciba | 0.30 | 0.30 | 0.30 | 0.30 |
| | Citrate | | | | | |
| TINOVIS ® ADE | Sodium Acrylates | Ciba | 0.10 | 0.10 | 0.10 | 0.10 |
| | Copolymer (and) | | | | | |
| | Hydrogenated Polydecene | | | | | |
| | (and) PPG-1 Trideceth-6 | | | | | |

Manufacturing Instruction
Heat up part A and B to 80° C. Adjust pH of B to around 6, then add A into B under high stirring speed (using an Ultra Turrax). Cool down under stirring, adjust pH to 7. Add part D under stirring. Homogenize again by around 50° C.
Below 40° C. add the ingredients of part E in the listed order. Adjust the final pH to around 7.0.
Alternatively, Random Terpolymer can be post-added to the system after it is formed.
**Addition of Random Terpolymer improves water-resistant properties of the formulation.

### Example 34

### Refreshing and Cooling Body Mist

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Dub DOA | Diethylhexyl Adipate | Stearinerie | 13.00 | 13.00 | 13.00 | 13.00 |
| | | Dubois Fils | | | | |
| Eusolex HMS | Homosalate | Merck | 15.00 | 15.00 | 15.00 | 15.00 |
| Neo Heliopan 303 | Octocrylene | Symrise | 10.00 | 10.00 | 10.00 | 10.00 |
| Neo Heliopan, | Ethylhexyl Salicylate | Symrise | 5.00 | 5.00 | 5.00 | 5.00 |
| Type OS | | | | | | |
| Parsol 1789 | Butyl | DSM | 3.00 | 3.00 | 3.00 | 3.00 |
| | Methoxydibenzoylmethane | | | | | |
| TINOSORB ® S | Bis-Ethylhexyloxyphenol | Ciba | 2.00 | 2.00 | 2.00 | 2.00 |
| | Methoxyphenyl Triazine | | | | | |
| Part B | | | | | | |
| Dow Corning | Cyclopentasiloxane (and) | Dow | 5.00 | 5.00 | 5.00 | 5.00 |
| 345 Fluid | Cyclohexasiloxane | Corning | | | | |
| Part C | | | | | | |
| Ethanol | 96% Alcohol | Berkel AHK | q.s. to 100% | q.s. to 100% | q.s. to 100% | q.s. to 100% |
| | Random Terpolymer | Ciba | 0.01 | 0.05 | 1.00 | 2.00 |
| | Example 13** | | | | | |

Manufacturing Instruction
Heat up part A to 80° C., when the ingredients are melted let stir until uniform..
Cool down, below 30° C., add part B and part C under stirring.
**Addition of Random Terpolymer improves water-resistant properties of the formulation.

### Example 35

### PEG and Silicon Free Sun Gel with 100% Particulate Filters Emulsifier free

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Emulfree CBG | Isostearyl Alcohol (and) | Gattefosse | 6 | 6 | 6 | 6 |
| | Butylene Glycol Cocoate | | | | | |
| | (and) Ethylcellulose | | | | | |
| Tegosoft CT | Caprylic/Capric | Evonik | 10 | 10 | 10 | 10 |
| | Triglyceride | | | | | |
| Cetiol V | Decyl Oleate | Cognis | 5 | 5 | 5 | 5 |
| Cocoate BG | Butylene Glycol Cocoate | Gattefossé | 3 | 3 | 3 | 3 |
| MT 150 EX | Titanium Dioxyde (and) | Tayca | 12 | 12 | 6 | 0 |
| | Stearic Acid (and) | | | | | |
| | Aluminium Hydroxyde | | | | | |
| | Zinc Oxide | | | | 6 | |
| Part B | | | | | | |
| Water | Water | | qs to 100% | qs to 100% | qs to 100% | qs to 100% |
| Glycerin 85% | Glycerin | Fluka | 5 | 5 | 5 | 5 |
| | Random Terpolymer | Ciba | 0.25 | 0.5 | 2.00 | 2.00 |
| | Example 13 ** | | | | | |
| Keltrol RD | Xanthan Gum | Rahn | 0.5 | 0.5 | 0.5 | 0.5 |
| Veegum Ultra | Magnesium Aluminium | R. T | 0.3 | 0.3 | 0.3 | 0.3 |
| | Silicate | Vanderbilt | | | | |
| TINODERM ®SG-P | Aqua (and) Pentylene | Ciba | 4 | 4 | 4 | 4 |
| | Glycol (and) Sclerotium | | | | | |
| | Gum | | | | | |
| Part C | | | | | | |
| TINOSORB ® M | Methylene Bis- | Ciba | 12 | | 4 | 8 |
| | Benzotriazolyl | | | | | |
| | Tetramethylbutylphenol | | | | | |
| | (and) Aqua (and) Decyl | | | | | |
| | Glucoside (and) | | | | | |
| | Propylene Glycol (and) | | | | | |
| | Xanthan Gum | | | | | |
| TINOSORB ® M-PGL | Methylene Bis- | Ciba | | 12 | | |
| | Benzotriazolyl | | | | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | | 2.00 | 3.00 |
| | 50% micronized active | | | | | |
| | in aqueous solution | | | | | |
| | Methanone, 1,1′-(1,4- | Ciba | | | 1.00 | 1.00 |
| | piperazinediyl) bis[1-[2- | | | | | |
| | [4-(diethylamino)-2- | | | | | |
| | hydroxybenzoyl]phenyl]- | | | | | |
| | (CAS No: 919803-06-8), | | | | | |
| | 50% micronized active | | | | | |
| | in aqueous solution | | | | | |
| Part D | | | | | | |
| Orgasol 2002 D | Nylon-12 | Atofina | 2 | 2 | 2 | 2 |
| NAT COS 20 | | | | | | |
| Microns | | | | | | |
| Techpolymer MB | Polymethyl Methacrylate | Sekisui | 2 | 2 | 2 | 2 |
| 8C | | | | | | |
| | Preservative | | Qs | qs | qs | qs |

Manufacturing Instructions
Heat part A without MT 150EX to 75° C. Add Titanium Dioxide and homogenize until homogeneous. Prepare part B: First Disperse Veegum Ultra into the water and heat up to 75° C., homogenize with an Ultra Turrax then add Xanthan Gum. When homogeneous, add the rest of part B. Add part A into part B under high speed homogeneization (Ultra Turrax). Cool down under stirring, finally add part C and the ingredients of D by 40° C. Alternatively, Random Terpolymer can be post-added to the system after gel is formed.
** Addition of Random Terpolymer improves water-resistant properties of the formulation.

### Example 36

### Gel Cream

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Water | Water | | q.s. to 100% | q.s. to 100% | q.s. to 100% | q.s. to 100% |
| Dissolvine NA-2-P | Disodium EDTA | Akzo Nobel | 0.20 | 0.20 | 0.20 | 0.20 |
| | Random | Ciba | 0.5 | 1.50 | 2.50 | 4.00 |
| | Terpolymer | | | | | |
| | Example 13** | | | | | |
| TINODERM ®SG-P | Aqua (and) Pentylene | Ciba | 2.00 | 2.00 | 2.00 | 2.00 |
| | Glycol (and) | | | | | |
| | Sclerotium Gum | | | | | |
| Part B | | | | | | |
| Eumulgin B 1 | Ceteareth-12 | Cognis | 1.00 | 1.00 | 1.00 | 1.00 |
| Cetiol B | Dibutyl Adipate | Cognis | 5.00 | 5.00 | 5.00 | 5.00 |
| Dermofeel TC-7 | Triheptanoin | Dr. | 3.00 | 3.00 | 3.00 | 3.00 |
| | | Straetmans | | | | |
| Uvinul A Plus | Diethylamino | BASF | 10.00 | 10.00 | 10.00 | 10.00 |
| | Hydroxybenzoyl | | | | | |
| | Hexyl Benzoate | | | | | |
| Neo Heliopan E | Isoamyl p- | Symrise | 7.50 | 7.50 | 7.50 | 7.50 |
| 1000 | Methoxycinnamate | | | | | |
| Uvinul T 150 | Ethylhexyl Triazone | BASF | 3.20 | 3.20 | 3.20 | 3.20 |
| TINOSORB ® S | Bis- | Ciba | 1.50 | 1.50 | 1.50 | 1.50 |
| | Ethylhexyloxyphenol | | | | | |
| | Methoxyphenyl | | | | | |
| | Triazine | | | | | |
| Part C | | | | | | |
| TINOVIS ® ADE | Sodium Acrylates | Ciba | 1.20 | 1.20 | 1.20 | 1.20 |
| | Copolymer (and) | | | | | |
| | Hydrogenated | | | | | |
| | Polydecene (and) | | | | | |
| | PPG-1 Trideceth-6 | | | | | |
| Part D | | | | | | |
| Dow Corning 246 | Cyclohexasiloxane | Dow Corning | 4.00 | 4.00 | 4.00 | 4.00 |
| Fluid | (and) | | | | | |
| | Cyclopentasiloxane | | | | | |
| Part E | | | | | | |
| Phenonip | Phenoxyethanol | Clariant | 1.00 | 1.00 | 1.00 | 1.00 |
| | (and) Methylparaben | | | | | |
| | (and) Ethylparaben | | | | | |
| | (and) Butylparaben | | | | | |
| | (and) Propylparaben | | | | | |
| | (and) | | | | | |
| | Isobutylparaben | | | | | |
| TINODERM ® E | Aqua (and) | Ciba | 0.50 | 0.50 | 0.50 | 0.50 |
| | Tocopheryl Acetate | | | | | |
| | (and) | | | | | |
| | Caprylic/Capric | | | | | |
| | Triglyceride (and) | | | | | |
| | Polysorbate 80 (and) | | | | | |
| | Lecithin | | | | | |

Manufacturing Instruction
Heat part A and B at 75° C. Under Ultra Turrax mixer, add part B (75° C.) into part A (75° C.).
Add immediately part C, mix until homogeneous. By 60° C., add part D and mix until homogeneous.
At room temperature, add part E and mix until homogeneous. Alternatively, Random Terpolymer can be post-added to the system after system is formed.
**Addition of Random Terpolymer improves water-resistant properties of the formulation.

### Example 37

### High UV Protection Sun Cream

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Cithrol 10MS | PEG-20 Stearate | Croda | 3.00 | 3.00 | 3.00 | 3.00 |
| Tegin M | Glyceryl Stearate | Evonik | 2.00 | 2.00 | 2.00 | 2.00 |
| Lanette 18 | Stearyl Alcohol | Cognis | 0.60 | 0.60 | 0.60 | 0.60 |
| Cetiol B | Dibutyl Adipate | Cognis | 5.00 | 5.00 | 5.00 | 5.00 |
| Uvinul A Plus | Diethylamino | BASF | 10.00 | 10.00 | 10.00 | 10.00 |
| | Hydroxybenzoyl | | | | | |
| | Hexyl Benzoate | | | | | |
| Neo Heliopan, | Octocrylene | Symrise | 9.00 | 9.00 | 9.00 | 9.00 |
| Type 303 | | | | | | |
| Parsol MCX | Ethylhexyl | DSM | 7.50 | 7.50 | 7.50 | 7.50 |
| | Methoxycinnamate | | | | | |
| TINOSORB ® S | Bis- | Ciba | 3.00 | 3.00 | 3.00 | 3.00 |
| | Ethylhexyloxyphenol | | | | | |
| | Methoxyphenyl | | | | | |
| | Triazine | | | | | |
| Part B | | | | | | |
| MT-100 Z T | Titanium Dioxide | Tayca | 4.00 | 4.00 | 4.00 | 4.00 |
| | (and) Aluminum | | | | | |
| | Hydroxide (and) | | | | | |
| | Stearic Acid | | | | | |
| Part C | | | | | | |
| Water | Water | | q.s.p 100% | q.s.p 100% | q.s.p 100% | q.s.p 100% |
| | Random Terpolymer | Ciba | 0.50 | 1.50 | 2.50 | 3.50 |
| | Example 13** | | | | | |
| Dissolvine NA-2-P | Disodium EDTA | Akzo Nobel | 0.20 | 0.20 | 0.20 | 0.20 |
| Sunsphere Powder | Styrene/Acrylates | ISP | 5.00 | 5.00 | 5.00 | 5.00 |
| | Copolymer | | | | | |
| Keltrol T | Xanthan Gum | Rahn | 0.50 | 0.50 | 0.50 | 0.50 |
| Amphisol K | Potassium Cetyl | DSM | 2.00 | 2.00 | 2.00 | 2.00 |
| | Phosphate | | | | | |
| Part D | | | | | | |
| Dow Corning 246 | Cyclohexasiloxane | Dow Corning | 3.00 | 3.00 | 3.00 | 3.00 |
| Fluid | (and) | | | | | |
| | Cyclopentasiloxane | | | | | |
| TINODERM ® E | Aqua (and) | Ciba | 0.50 | 0.50 | 0.50 | 0.50 |
| | Tocopheryl Acetate | | | | | |
| | (and) Caprylic/Capric | | | | | |
| | Triglyceride (and) | | | | | |
| | Polysorbate 80 (and) | | | | | |
| | Lecithin | | | | | |
| | Preservative | | q.s | q.s | q.s | q.s |

Manufacturing Instruction
Heat part A at 75° C. until homogeneous.
Add part B into part A and mix under quick Ultra Turrax type mixer until homogeneous.
Heat part C without Sunsphere Powder, Keltrol T and Amphisol K at 75° C.
At 75° C. under quick Ultra Turrax mixer, add Sunsphere Powder, Keltrol T and Amphisol K and mix until homogeneous.
Add A + B at 75° C. into C at 75° C. and homogenize.
Below 60° C., add the ingredients of part D and mix until homogeneous.
Cool down to room temperature under stirrer.
**Addition of Random Terpolymer improves water-resistant properties of the formulation.

### Example 38

### Dry Sensation Cream

Fast penetrating, light end skin feeling

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Sensanov WR | C20-22 Alkyl | Seppic | 4.00 | 4.00 | 4.00 | 4.00 |
| | Phosphate (and) C20- | | | | | |
| | C22 Alcohols | | | | | |
| Cetiol B | Dibutyl Adipate | Cognis | 4.00 | 4.00 | 4.00 | 4.00 |
| Uvinul A Plus | Diethylamino | BASF | 10.00 | 10.00 | 10.00 | 10.00 |
| | Hydroxybenzoyl | | | | | |
| | Hexyl Benzoate | | | | | |
| Parsol MCX | Ethylhexyl | DSM | 7.50 | 7.50 | 7.50 | 7.50 |
| | Methoxycinnamate | | | | | |
| TINOSORB ® S | Bis- | Ciba | 2.00 | 2.00 | 2.00 | 2.00 |
| | Ethylhexyloxyphenol | | | | | |
| | Methoxyphenyl | | | | | |
| | Triazine | | | | | |
| Uvinul T 150 | Ethylhexyl Triazone | BASF | 1.00 | 1.00 | 1.00 | 1.00 |
| Part B | | | | | | |
| Water | Water | | q.s.p 100% | q.s.p 100% | q.s.p 100% | q.s.p 100% |
| Dissolvine NA-2-P | Disodium EDTA | Akzo Nobel | 0.20 | 0.20 | 0.20 | 0.20 |
| TINODERM ®SG-P | Aqua (and) Pentylene | Ciba | 2.00 | 2.00 | 2.00 | 2.00 |
| | Glycol (and) | | | | | |
| | Sclerotium Gum | | | | | |
| Tris Amino Ultra | Tromethamine | Angus | 0.54 | 0.54 | 0.54 | 0.54 |
| Pur | | Chemie | | | | |
| | | GmbH | | | | |
| Part C | | | | | | |
| Dow Corning 246 | Cyclohexasiloxane | Dow | 5.00 | 5.00 | 5.00 | 5.00 |
| Fluid | (and) | Corning | | | | |
| | Cyclopentasiloxane | | | | | |
| Micropearl M305 | Methylmethacrylate | Seppic | 2.00 | 2.00 | 2.00 | 2.00 |
| | Crosspolymer | | | | | |
| Part D | | | | | | |
| TINOVIS ® ADE | Sodium Acrylates | Ciba | 0.40 | 0.40 | 0.40 | 0.40 |
| | Copolymer (and) | | | | | |
| | Hydrogenated | | | | | |
| | Polydecene (and) | | | | | |
| | PPG-1 Trideceth-6 | | | | | |
| | Random Terpolymer | Ciba | 0.75 | 1.75 | 2.75 | 3.75 |
| | Example 13** | | | | | |
| Part E | | | | | | |
| Phenonip | Phenoxyethanol (and) | Clariant | 1.00 | 1.00 | 1.00 | 1.00 |
| | Methylparaben (and) | | | | | |
| | Ethylparaben (and) | | | | | |
| | Butylparaben (and) | | | | | |
| | Propylparaben (and) | | | | | |
| | Isobutylparaben | | | | | |
| TINODERM ® E | Aqua (and) | Ciba | 0.50 | 0.50 | 0.50 | 0.50 |
| | Tocopheryl Acetate | | | | | |
| | (and) Caprylic/Capric | | | | | |
| | Triglyceride (and) | | | | | |
| | Polysorbate 80 (and) | | | | | |
| | Lecithin | | | | | |

Manufacturing Instruction
Heat up part A and B to 75° C. until homogeneous.
Add part A (75° C.) into part B (75° C.) and homogenize.
By 60° C., add part C and mix until homogeneous. Add part D and homogenize.
Cool down to room temperature, add part E and mix until homogeneous.
**Addition of Random Terpolymer improves water-resistant properties of the formulation.

### Example 39

### UV Defense Spray

Non greasy

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Lanette 16 | Cetyl Alcohol | Cognis | 0.30 | 0.30 | 0.30 | 0.30 |
| Tegosoft OS | Ethylhexyl Stearate | Evonik | 4.00 | 4.00 | 4.00 | 4.00 |
| Neo Heliopan, | Octocrylene Symrise | 10.00 | | | | |
| Type 303 | | | | | | |
| Neo Heliopan, | Ethylhexyl Salicylate | Symrise | 5.00 | 5.00 | 5.00 | 5.00 |
| Type OS | | | | | | |
| Eusolex T-AVO | Titanium Dioxide (and) | Merck | 3.30 | 3.30 | 3.30 | 3.30 |
| | Silica | | | | | |
| Parsol 1789 | Butyl | DSM | 2.90 | 2.90 | 2.90 | 2.90 |
| | Methoxydibenzoylmethane | | | | | |
| TINOSORB ® S | Bis-Ethylhexyloxyphenol | Ciba | 1.50 | 1.50 | 1.50 | 1.50 |
| | Methoxyphenyl Triazine | | | | | |
| Part B | | | | | | |
| Water | Water | | qs to 100% | qs to 100% | qs to 100% | qs to 100% |
| | Random Terpolymer | Ciba | 1.00 | 2.00 | 1.00 | 2.00 |
| | Example 13** | | | | | |
| Glycerin 85% | Glycerin | Fluka | 5.00 | 5.00 | 5.00 | 5.00 |
| Amphisol A | Cetyl Phosphate | DSM | 3.00 | 3.00 | 3.00 | 3.00 |
| Avicel PC611 | Microcrystalline Cellulose | FMC | 1.50 | 1.50 | 1.50 | 1.50 |
| | | Corporation | | | | |
| Dissolvine NA-2P | Disodium EDTA | Akzo Nobel | 0.20 | 0.20 | 0.20 | 0.20 |
| Part C | | | | | | |
| Triethanolamine | Triethanolamine | Riedel | 1.00 | 1.00 | 1.00 | 1.00 |
| Part D | | | | | | |
| TINOSORB ® M | Methylene Bis- | Ciba | 4.00 | | 2.00 | |
| | Benzotriazolyl | | | | | |
| | Tetramethylbutylphenol | | | | | |
| | (and) Aqua (and) Decyl | | | | | |
| | Glucoside (and) Propylene | | | | | |
| | Glycol (and) Xanthan Gum | | | | | |
| TINOSORB ® M-PGL | Methylene Bis- | Ciba | | 4.00 | | |
| | Benzotriazolyl | | | | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | | 2.00 | 3.00 |
| | 50% micronized active in | | | | | |
| | aqueous solution | | | | | |
| | Methanone, 1,1′-(1,4- | Ciba | | | 1.00 | 1.00 |
| | piperazinediyl) bis[1-[2-[4- | | | | | |
| | (diethylamino)-2- | | | | | |
| | hydroxybenzoyl]phenyl]- | | | | | |
| | (CAS No: 919803-06-8), | | | | | |
| | 50% micronized active in | | | | | |
| | aqueous solution | | | | | |
| TINODERM ® E | Aqua (and) Tocopheryl | Ciba | 3.00 | 3.00 | 3.00 | 3.00 |
| | Acetate (and) | | | | | |
| | Caprylic/Capric | | | | | |
| | Triglyceride (and) | | | | | |
| | Polysorbate 80 (and) | | | | | |
| | Lecithin | | | | | |
| Dow Corning 245 | Cyclopentasiloxane | Dow Corning | 2.00 | 2.00 | 2.00 | 2.00 |
| Fluid | | | | | | |
| | Preservative | | qs | qs | qs | qs |

Manufacturing Instruction
Heat part A to 75° C., add Titanium Dioxide, homogenize.
Prepare part B: first disperse Avicel PC611 into water, heat up to 75° C., stir.
When well dispersed, add the rest of the ingredients of part B without Amphisol A.
Then add Amphisol A, stir slowly 10 more minutes.
Add part A into part B (by 75° C.) under stirring, homogenize.
Adjust pH value with part C.
Cool down to room temperature, below 50° C. add the ingredients of part D in the listed order.
**Addition of Random Terpolymer improves water-resistant properties of the formulation.

### Example 40

### Global Sun Solution

| | | | % w/w | % w/w | % w/w | % w/w |
|---|---|---|---|---|---|---|
| Composition | INCI-Name/Chemical- | | (as | (as | (as | (as |
| Trade Name | Name | Supplier | supplied) | supplied) | supplied) | supplied) |
| Part A | | | | | | |
| Emulgade SE/PF | Glyceryl Stearate (and) | Cognis | 6.00 | 6.00 | 6.00 | 6.00 |
| | Ceteareth-20 (and) | | | | | |
| | Ceteareth-12 (and) | | | | | |
| | Cetearyl Alcohol (and) | | | | | |
| | Cetyl Palmitate | | | | | |
| Lanette 16 | Cetyl Alcohol | Cognis | 1.50 | 1.50 | 1.50 | 1.50 |
| Lanette 18 | Stearyl Alcohol | Cognis | 1.00 | 1.00 | 1.00 | 1.00 |
| Cetiol CC | Dicaprylyl Carbonate | Cognis | 3.00 | 3.00 | 3.00 | 3.00 |
| Neo Heliopan HMS | Homosalate | Symrise | 10.00 | 10.00 | 10.00 | 10.00 |
| Neo Heliopan, | Octocrylene | Symrise | 10.00 | 10.00 | 10.00 | 10.00 |
| Type 303 | | | | | | |
| Neo Heliopan, | Ethylhexyl Salicylate | Symrise | 5.00 | 5.00 | 5.00 | 5.00 |
| Type OS | | | | | | |
| Parsol 1789 | Butyl | DSM | 3.00 | 3.00 | 3.00 | 3.00 |
| | Methoxydibenzoylmethane | | | | | |
| TINOSORB ® S | Bis-Ethylhexyloxyphenol | Ciba | 1.80 | 1.80 | 1.80 | 1.80 |
| | Methoxyphenyl Triazine | | | | | |
| Part B | | | | | | |
| Water | Water | | qs to 100% | qs to 100% | qs to 100% | qs to 100% |
| Butylene Glycol | Butylene Glycol | Hoechst | 5.00 | 5.00 | 5.00 | 5.00 |
| | | Celanese | | | | |
| TINODERM ®SG-P | Pentylene Glycol (and) | Ciba | 2.00 | 2.00 | 2.00 | 2.00 |
| | Sclerotium Gum | | | | | |
| Keltrol T | Xanthan Gum | Rahn | 0.40 | 0.40 | 0.40 | 0.40 |
| Dissolvine NA-2 | Disodium EDTA | Akzo Nobel | 0.20 | 0.20 | 0.20 | 0.20 |
| Part C | | | | | | |
| Dow Corning 245 | Cyclopentasiloxane | Dow | 3.00 | 3.00 | 3.00 | 3.00 |
| Fluid | | Corning | | | | |
| Velvesil 125 | Cyclopentasiloxane (and) | Momentive | 1.00 | 1.00 | 1.00 | 1.00 |
| | C30-45 Alkyl Cetearyl | | | | | |
| | Dimethicone | | | | | |
| | Crosspolymer | | | | | |
| Part D | | | | | | |
| TINOSORB ® M | Methylene Bis- | Ciba | 4.00 | | 4.00 | 4.00 |
| | Benzotriazolyl | | | | | |
| | Tetramethylbutylphenol | | | | | |
| | (and) Aqua (and) Decyl | | | | | |
| | Glucoside (and) Propylene | | | | | |
| | Glycol (and) Xanthan Gum | | | | | |
| TINOSORB ® M-PGL | Methylene Bis-Benzotriazolyl | Ciba | | 4.00 | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | | 2.00 | 1.00 |
| | 50% micronized active in | | | | | |
| | aqueous solution | | | | | |
| | Methanone, 1,1′-(1,4- | Ciba | | 1.00 | 1.00 | |
| | piperazinediyl) bis[1-[2-[4- | | | | | |
| | (diethylamino)-2- | | | | | |
| | hydroxybenzoyl]phenyl]- | | | | | |
| | (CAS No: 919803-06-8), | | | | | |
| | 50% micronized active in | | | | | |
| | aqueous solution | | | | | |
| | Random Terpolymer | Ciba | 2.00 | 2.00 | 2.00 | 2.00 |
| | Example 13** | | | | | |
| Mackaderm Asto- | Aluminium Starch | Mc Intyre | 3.00 | 3.00 | 3.00 | 3.00 |
| Dry | Octenylsuccinate | | | | | |
| TINODERM ® E | Aqua (and) Tocopheryl | Ciba | 2.00 | 2.00 | 2.00 | 2.00 |
| | Acetate (and) | | | | | |
| | Caprylic/Capric | | | | | |
| | Triglyceride (and) | | | | | |
| | Polysorbate 80 (and) | | | | | |
| | Lecithin | | | | | |
| Nipaguard MPA | Benzyl Alcohol (and) | Clariant | 1.00 | 1.00 | 1.00 | 1.00 |
| | Methylparaben (and) | | | | | |
| | Propylparaben | | | | | |

Manufacturing instruction
Heat up part A and B to 75° C.
Add part A into part B, then homogenize with an Ultra Turrax.
Mix part C. Add part C by 60° C. under stirring, homogenize with an Ultra Turrax.
Below 50° C., add the ingredients of part D in the listed order.
Cool down to room temperature under continuous stirring.
**Addition of Random Terpolymer improves water-resistant properties of the formulation.

### Example 41

### Total Effect Lightening Face Gel Cream

Cold process
Emerging Market

| Composition | INCI-Name/Chemical- | | | | | |
|---|---|---|---|---|---|---|
| Trade Name | Name | Supplier | % w/w (as supplied) | | | |
| Part A | | | | | | |
| Tegosoft P | Isopropyl Palmitate | Evonik | 5.00 | 5.00 | 5.00 | 5.00 |
| Parsol MCX | Ethylhexyl | DSM | 7.00 | 7.00 | 7.00 | 7.00 |
| | Methoxycinnamate | | | | | |
| Part B | | | | | | |
| Water | Water | | qs to 100 | qs to 100 | qs to 100 | qs to 100 |
| Glycerin 85% | Glycerin | Fluka | 5.00 | 5.00 | 5.00 | 5.00 |
| Nicotinamide | Niacinamide | Merck | 0.50 | 0.50 | 0.50 | 0.50 |
| | | (Calbiochem) | | | | |
| Keltrol T | Xanthan Gum | Rahn | 0.20 | 0.20 | 0.20 | 0.20 |
| Dissolvine NA-2 | Disodium EDTA | Akzo Nobel | 0.10 | 0.10 | 0.10 | 0.10 |
| TINODERM ®SG-P | Pentylene Glycol (and) | Ciba | 2.00 | 2.00 | 2.00 | 2.00 |
| | Sclerotium Gum | | | | | |
| Part C | | | | | | |
| TINOVIS ® ADE | Sodium Acrylates | Ciba | 1.25 | 1.25 | 1.25 | 1.25 |
| | Copolymer (and) | | | | | |
| | Hydrogenated | | | | | |
| | Polydecene (and) PPG-1 | | | | | |
| | Trideceth-6 | | | | | |
| Part D | | | | | | |
| TINOSORB ® M | Methylene Bis- | Ciba | 3.00 | | | 2.00 |
| | Benzotriazolyl | | | | | |
| | Tetramethylbutylphenol | | | | | |
| | (and) Aqua (and) Decyl | | | | | |
| | Glucoside (and) | | | | | |
| | Propylene Glycol (and) | | | | | |
| | Xanthan Gum | | | | | |
| TINOSORB ® M-PGL | Methylene Bis- | Ciba | | 3.00 | | |
| | Benzotriazolyl | | | | | |
| | Tetramethylbutylphenol | | | | | |
| | (and) Aqua (and) | | | | | |
| | Polyglyceryl 10-Laurate | | | | | |
| | (and) Butylene Glycol | | | | | |
| | Tris-Biphenyl Triazine, | Ciba | | | 2.00 | |
| | 50% micronized active | | | | | |
| | in aqueous solution | | | | | |
| | Methanone, 1,1′-(1,4- | Ciba | | | 1.00 | 1.00 |
| | piperazinediyl) bis[1-[2- | | | | | |
| | [4-(diethylamino)-2- | | | | | |
| | hydroxybenzoyl]phenyl]- | | | | | |
| | (CAS No: 919803-06-8), | | | | | |
| | 50% micronized active | | | | | |
| | in aqueous solution | | | | | |
| | Random Terpolymer | Ciba | 1.00 | 1.00 | 1.00 | 1.00 |
| | Example 13** | | | | | |
| Dry- Flo PC | Aluminum Starch | National Starch | 1.00 | 1.00 | 1.00 | 1.00 |
| | Octenylsuccinate | | | | | |
| Phenonip | Phenoxyethanol (and) | Clariant | 1.00 | 1.00 | 1.00 | 1.00 |
| | Methylparaben (and) | | | | | |
| | Ethylparaben (and) | | | | | |
| | Butylparaben (and) | | | | | |
| | Propylparaben (and) | | | | | |
| | Isobutylparaben | | | | | |

Manufacturing Instruction
Add part A to part B under stirring. Homogenize for a short time with an Ultra Turrax.
Add part C under stirring.
When homogeneous add the ingredients of part D in the listed order.
**Addition of Random Terpolymer improves water-resistant properties of the formulation.

What is claimed:

1. A sunscreen composition comprising
A) at least one random terpolymer of formula (Ia) 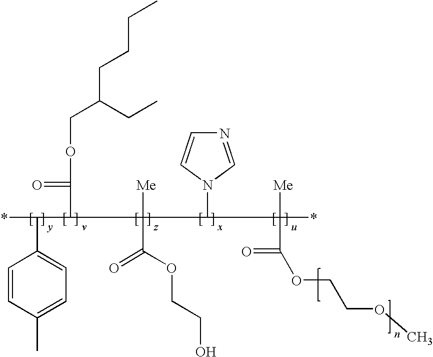
wherein
y, v, u, z and x represent the percentage by weight that each repeating unit or derived monomer is contained within the terpolymer;
y, v, u, z and x add up to total 100 weight percent relative to the total weight of the terpolymer;
y is from about 1 to about 30% by weight of the terpolymer;
v is from about 5% to about 75% by weight of the terpolymer by weight of the terpolymer u is from about 20% to about 80% by weight of the terpolymer;
z is from about 1% to about 40% by weight of the terpolymer;
x is from about 5% to about 25% by weight of the terpolymer;
* is a terminal group;
n is 2 to 20;
wherein formula (I) is characterized by a weight average molecular weight of from about 5,000 to about 50,000 Daltons;
and
B) a sunscreen is selected from group consisting of
b₁ at least one sparingly soluble micronized organic UV absorber,
b₂ Bis-Ethylhexyloxyphenol Methoxyphenyl Triazine,
b₃ at least one oil soluble organic UV absorber,
b₄ at least one inorganic UV absorber,
b₅ at least one water soluble UV absorber,
and
mixtures thereof.

2. The sunscreen composition according to claim 1, wherein
b₁) the at least one sparingly soluble micronized UV absorber is selected from the group consisting of:
Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- and mixtures thereof;
b₂) is Bis-Ethylhexyloxyphenol Methoxyphenyl Triazine;
b₃) the at least one oil soluble organic UV absorber is selected from the group consisting of:
Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, Diethylamino Hydroxy Benzoyl Hexyl Benzoate (DHHB) and mixtures thereof;
b₄) the at least one inorganic UV absorber is selected from the group consisting of: titanium oxide, zinc oxide and mixtures thereof;
and
b₅) the at least one water soluble UV absorber is selected from the group consisting of Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, PEG-25 PABA and mixtures thereof.

3. The sunscreen composition according to claim 1, wherein component B) comprises both b₁ and b₂.

4. The sunscreen composition according to claim 1, wherein the component B) comprises (b₄) titanium dioxide or zinc oxide with the proviso that the sunscreen composition contains substantially no organic UV absorbers.

5. A sunscreen composition comprising
A) at least one random terpolymer of formula (I) 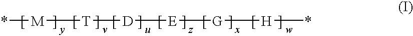
wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
x is from about 1% to about 50% by weight of the terpolymer;
w is from about 0% to about 50% by weight of the terpolymer;
is a terminal group, for example, a catalyst residue;
M, T, D, E, G, and H are covalently bonded to each other;
M is derived from at least one monomer of formula (II) 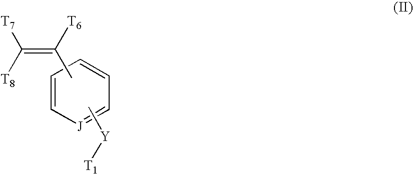
wherein T6, T7, and T8 are C1-C4 alkyl or hydrogen; Y is a direct bond, —O—, —S—, —N(H)— or —N(T1)-; T1 is hydrogen or C1-C4 alkyl; and J is a nitrogen or carbon atom;
T, D, and E are independently derived from at least one monomer of formula (III) 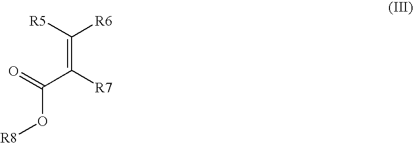
wherein R5, R6 and R7 may be the same or different and represent hydrogen or C1-C22 alkyl;
R8 is C1-C30 alkyl, C6-C15 cycloalkyl, or C6-C15 aryl; said substituted alkyl, said cycloalkyl or said aryl may also be substituted by one or more —OH and/or NH₂ groups; or said alkyl or said cycloalkyl may be interrupted by one or more —O— groups and/or N(H)— groups;
G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, and acrylonitrile;
(B) at least one UV screening agent;
and
(C) other cosmetically acceptable ingredients,
with the proviso that T, D, and E are different from each other,
wherein the component B comprises the UV absorber combination selected from the group of UV absorber combinations consisting of
Methylene Bis-Benzotriazolyl Tetramethylbutylphenol/Tris-Biphenyl Triazine/Methanone, 1,1′-(1,4-piperazinediyl)bis[1-[2-[4-(diethylamino)-2-hydroxybenzoyl]phenyl]-
and
Methylene Bis-Benzotriazolyl Tetramethylbutylphenol/Tris-Biphenyl Triazine/Methanone, 1,1′-(1,4-piperazinediyl)bis[1-[2-[4-(diethylamino)-2-hydroxybenzoyl]phenyl]-/Titanium oxide,
with the proviso that the sunscreen composition contains substantially no soluble organic UV absorber,
and
Methylene Bis-Benzotriazolyl Tetramethylbutylphenol, Tris-Biphenyl Triazine and Methanone, 1,1′-(1,4-piperazinediyl)bis[1-[2-[4-(diethylamino)-2-hydroxybenzoyl]phenyl]- are sparingly soluble micronized UV absorbers.

6. A sunscreen composition comprising
(A) at least one random terpolymer of formula (I) 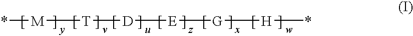
wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
x is from about 1% to about 50% by weight of the terpolymer;
w is from about 0% to about 50% by weight of the terpolymer;
* is a terminal group, for example, a catalyst residue;
M, T, D, E, G, and H are covalently bonded to each other;
M is derived from at least one monomer of formula (II) 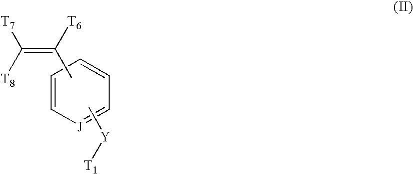
wherein T6, T7, and T8 are C1-C4 alkyl or hydrogen; Y is a direct bond, —O—, —S—, —N(H)— or —N(T1)-; T1 is hydrogen or C1-C4 alkyl; and J is a nitrogen or carbon atom;
T, D, and E are independently derived from at least one monomer of formula (III) 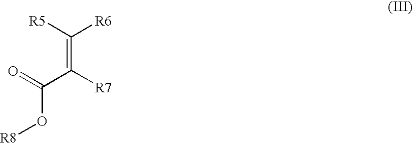
wherein R5, R6 and R7 may be the same or different and represent hydrogen or C1-C22 alkyl;
R8 is C1-C30 alkyl, C6-C15 cycloalkyl, or C6-C15 aryl; said substituted alkyl, said cycloalkyl or said aryl may also be substituted by one or more —OH and/or NH₂ groups;
or said alkyl or said cycloalkyl may be interrupted by one or more —O— groups and/or —N(H)— groups;
G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, and acrylonitrile;
and
(B) at least one UV screening agent;
(C) other cosmetically acceptable ingredients,
with the proviso that T, D, and E are different from each other,
wherein component B) comprises a UV absorber combination comprising
i.) 0.1 to 20 wt. % based on the total weight of the sunscreen composition selected from group consisting of
Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- and Bis-Ethylhexyloxyphenol Methoxyphenyl Triazine;
and
ii.) 0.1 to 20 wt. % based on the total weight of the sunscreen composition of a UV absorber selected from group consisting of Diethylamino Hydroxy Benzoyl Hexyl Benzoate (DHHB), Diethylhexyl Butamido Triazone (DBT), Ethylhexyl Methoxycinnamate (EHMC), Ethylhexyl Salicylate (EHS), Ethylhexyl Triazone (EHT), Octocrylene (OCR), Butyl Methoxydibenzoylmethane (BMBM), Titanium Dioxide and Phenylbenzimidazole Sulfonic Acid (PBSA).
wherein, Methylene Bis-Benzotriazolyl Tetramethylbutylphenol, Tris-Biphenyl Triazine, Methanone, 1,1-(1,4-piperazinediyl)bis[1-[2-[4-(diethylamino)-2-hydroxybenzoyl]phenyl]- are sparingly soluble micronized UV absorbers.

7. The sunscreen composition according to claim 6, wherein the
component B) comprises a UV absorber combination selected from the group of combinations comprising
Bis-Ethylhexyloxyphenol Methoxyphenyl Triazine/Methylene Bis-Benzotriazolyl Tetramethylbutylphenol/Butyl Methoxydibenzoylmethane (BMBM);
Bis-Ethylhexyloxyphenol Methoxyphenyl Triazine/Butyl Methoxydibenzoylmethane (BMBM);
Methylene Bis-Benzotriazolyl Tetramethylbutylphenol/Butyl Methoxydibenzoylmethane (BMBM);
Bis-Ethylhexyloxyphenol Methoxyphenyl Triazine/Methylene Bis-Benzotriazolyl Tetramethylbutylphenol/Ethylhexyl Methoxycinnamate (EHMC);
Bis-Ethylhexyloxyphenol Methoxyphenyl Triazine/Ethylhexyl Methoxycinnamate (EHMC);
Methylene Bis-Benzotriazolyl Tetramethylbutylphenol/Ethylhexyl Methoxycinnamate (EHMC);
Bis-Ethylhexyloxyphenol Methoxyphenyl Triazine/Methylene Bis-Benzotriazolyl Tetramethylbutylphenol/Octocrylene (OCR);
Bis-Ethylhexyloxyphenol Methoxyphenyl Triazine/Octocrylene (OCR);
Methylene Bis-Benzotriazolyl Tetramethylbutylphenol/Octocrylene (OCR);
Bis-Ethylhexyloxyphenol Methoxyphenyl Triazine/Methylene Bis-Benzotriazolyl Tetramethylbutylphenol/Phenylbenzimidazole Sulfonic Acid (PBSA);
Bis-Ethylhexyloxyphenol Methoxyphenyl Triazine/Phenylbenzimidazole Sulfonic Acid (PBSA);
Methylene Bis-Benzotriazolyl Tetramethylbutylphenol/Phenylbenzimidazole Sulfonic Acid (PBSA);
Bis-Ethylhexyloxyphenol Methoxyphenyl Triazine/Methylene Bis-Benzotriazolyl Tetramethylbutylphenol/Ethylhexyl Salicylate (EHS);
Bis-Ethylhexyloxyphenol Methoxyphenyl Triazine/Ethylhexyl Salicylate (EHS);
Methylene Bis-Benzotriazolyl Tetramethylbutylphenol/Ethylhexyl Salicylate (EHS);
Bis-Ethylhexyloxyphenol Methoxyphenyl Triazine/Methylene Bis-Benzotriazolyl Tetramethylbutylphenol/Diethylhexyl Butamido Triazone (DBT);
Bis-Ethylhexyloxyphenol Methoxyphenyl Triazine/Diethylhexyl Butamido Triazone (DBT);
Methylene Bis-Benzotriazolyl Tetramethylbutylphenol/Diethylhexyl Butamido Triazone (DBT);
Bis-Ethylhexyloxyphenol Methoxyphenyl Triazine/Methylene Bis-Benzotriazolyl Tetramethylbutylphenol/Titanium Dioxide;
Bis-Ethylhexyloxyphenol Methoxyphenyl Triazine/Titanium Dioxide;
and
Methylene Bis-Benzotriazolyl Tetramethylbutylphenol/Titanium Dioxide
wherein, Benzotriazolyl Tetramethylbutylphenol is a sparingly soluble micronized UV absorber.

8. The sunscreen composition according to anyone of claim 6, wherein the component B) comprises a UV absorber combination selected from the group of combinations comprising
Methylene Bis-Benzotriazolyl Tetramethylbutylphenol/Bis-Ethylhexyloxyphenol Methoxyphenyl Triazine/Diethylamino Hydroxy Benzoyl Hexyl Benzoate (DHHB);
Methylene Bis-Benzotriazolyl Tetramethylbutylphenol/Diethylamino Hydroxy Benzoyl Hexyl Benzoate (DHHB);
Bis-Ethylhexyloxyphenol Methoxyphenyl Triazine/Diethylamino Hydroxy Benzoyl Hexyl Benzoate (DHHB);
Methylene Bis-Benzotriazolyl Tetramethylbutylphenol/Diethylamino Hydroxy Benzoyl Hexyl Benzoate (DHHB)/Ethylhexyl Methoxycinnamate (EHMC);
Bis-Ethylhexyloxyphenol Methoxyphenyl Triazine/Diethylamino Hydroxy Benzoyl Hexyl Benzoate (DHHB)/Ethylhexyl Methoxycinnamate (EHMC);
Methylene Bis-Benzotriazolyl Tetramethylbutylphenol/Bis-Ethylhexyloxyphenol Methoxyphenyl Triazine/Diethylamino Hydroxy Benzoyl Hexyl Benzoate (DHHB)/Ethylhexyl Methoxycinnamate (EHMC);
wherein, Methylene Bis-Benzotriazolyl Tetramethylbutylphenol is a sparingly soluble micronized UV absorber.

9. The sunscreen composition according to claim 1, wherein
component B) comprises a UV absorber combination comprising
i.) 0.1 to 20 wt. % based on the total weight of the sunscreen composition of a UV absorber selected from group consisting of Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- and Bis-Ethylhexyloxyphenol Methoxyphenyl Triazine;
wherein Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phenyl]- are sparingly soluble micronized UV absorbers;
ii.) 0.1 to 20 wt. % based on the total weight of the sunscreen composition of the UV absorber Butyl Methoxydibenzoylmethane (BMBM);
iii.) 0.1 to 20 wt. % based on the total weight of the sunscreen composition of the UV absorber selected from group consisting of
Ethylhexyl Triazone (EHT) and Octocrylene (OCR);
and
iv) 0 to 20 wt. % based on the total weight of the sunscreen composition of
Ethylhexyl Methoxycinnamate (EHMC),
Ethylhexyl Salicylate (EHS),
Diethylhexyl Butamido Triazone (DBT),
Phenylbenzimidazole Sulfonic Acid (PBSA)
or
Titanium dioxide.

10. The sunscreen composition according to claim 9, wherein
the
component B) comprises a UV absorber combination selected from the group of combinations comprising
Methylene Bis-Benzotriazolyl Tetramethylbutylphenol/Bis-Ethylhexyloxyphenol Methoxyphenyl Triazine/Butyl Methoxydibenzoylmethane (BMBM)/Octocrylene;
Methylene Bis-Benzotriazolyl Tetramethylbutylphenol/Butyl Methoxydibenzoylmethane (BMBM)/Octocrylene;
Bis-Ethylhexyloxyphenol Methoxyphenyl Triazine/Butyl Methoxydibenzoylmethane (BMBM)/Octocrylene;
Methylene Bis-Benzotriazolyl Tetramethylbutylphenol/Bis-Ethylhexyloxyphenol Methoxyphenyl Triazine/Butyl Methoxydibenzoylmethane (BMBM)/Ethylhexyl Triazone (EHT);
Methylene Bis-Benzotriazolyl Tetramethylbutylphenol/Butyl Methoxydibenzoylmethane (BMBM)/Ethylhexyl Triazone (EHT);
Bis-Ethylhexyloxyphenol Methoxyphenyl Triazine/Butyl Methoxydibenzoylmethane (BMBM)/Ethylhexyl Triazone (EHT);
Methylene Bis-Benzotriazolyl Tetramethylbutylphenol/Bis-Ethylhexyloxyphenol Methoxyphenyl Triazine/Butyl Methoxydibenzoylmethane (BMBM)/Octocrylene/Ethylhexyl Methoxycinnamate (EHMC);
Methylene Bis-Benzotriazolyl Tetramethylbutylphenol/Butyl Methoxydibenzoylmethane (BMBM)/Octocrylene/Ethylhexyl Methoxycinnamate (EHMC);
Bis-Ethylhexyloxyphenol Methoxyphenyl Triazine/Butyl Methoxydibenzoylmethane (BMBM)/Octocrylene/Ethylhexyl Methoxycinnamate (EHMC);
Methylene Bis-Benzotriazolyl Tetramethylbutylphenol/Bis-Ethylhexyloxyphenol Methoxyphenyl Triazine/Butyl Methoxydibenzoylmethane (BMBM)/Ethylhexyl Triazone (EHT)/Ethylhexyl Methoxycinnamate (EHMC);
Methylene Bis-Benzotriazolyl Tetramethylbutylphenol/Butyl Methoxydibenzoylmethane (BMBM)/Ethylhexyl Triazone (EHT)/Ethylhexyl Methoxycinnamate (EHMC);
Bis-Ethylhexyloxyphenol Methoxyphenyl Triazine/Butyl Methoxydibenzoylmethane (BMBM)/Ethylhexyl Triazone (EHT)/Ethylhexyl Methoxycinnamate (EHMC);
Methylene Bis-Benzotriazolyl Tetramethylbutylphenol/Bis-Ethylhexyloxyphenol Methoxyphenyl Triazine/Butyl Methoxydibenzoylmethane (BMBM)/Octocrylene/Ethylhexyl Salicylate (EHS);
Methylene Bis-Benzotriazolyl Tetramethylbutylphenol/Butyl Methoxydibenzoylmethane (BMBM)/Octocrylene/Ethylhexyl Salicylate (EHS);
Bis-Ethylhexyloxyphenol Methoxyphenyl Triazine/Butyl Methoxydibenzoylmethane (BMBM)/Octocrylene/Ethylhexyl Salicylate (EHS);
Methylene Bis-Benzotriazolyl Tetramethylbutylphenol/Bis-Ethylhexyloxyphenol Methoxyphenyl Triazine/Butyl Methoxydibenzoylmethane (BMBM)/Ethylhexyl Triazone (EHT)/Ethylhexyl Salicylate (EHS);
Methylene Bis-Benzotriazolyl Tetramethylbutylphenol/Butyl Methoxydibenzoylmethane (BMBM)/Ethylhexyl Triazone (EHT)/Ethylhexyl Salicylate (EHS);
Bis-Ethylhexyloxyphenol Methoxyphenyl Triazine/Butyl Methoxydibenzoylmethane (BMBM)/Ethylhexyl Triazone (EHT)/Ethylhexyl Salicylate (EHS);
wherein Methylene Bis-Benzotriazolyl Tetramethylbutylphenol is an sparingly soluble micronized UV absorber.

11. The sunscreen composition according to claims 1, wherein
the sparingly soluble micronized particle has a mean diameter size of about 0.01 to about 2 μm.

12. The sunscreen composition according to claim 1, wherein the sparingly soluble micronized UV absorber is obtained by grinding an organic UV filter in the presence of an aid which aid is selected from the group consisting of Decyl Glucoside, Polyglyceryl-10 Laurate, Sodium Myreth Sulfate and Sodium Stearoyl Glutamate.

13. The sunscreen composition according to claim 1 which further includes one or more further components selected from the group consisting of emollients, skin moisturisers, skin tanning accelerators, antioxidants, emulsion stabilisers, thickening agents, moisture retention agents, film formers, preservatives, perfumes, photostabilisers and colourants.

14. The sunscreen compositions according to claim 13, where the photostabiliser is selected from the group consisting of: Tris(Tetramethylhydroxypiperidinol) Citrate, Benzotriazolyl Dodecyl p-Cresol, Butyloctyl Salicylate, Diethylhexyl 2,6-Naphthalate and Polyester-8, Diethylhexyl Syringylidenemalonate.

15. The sunscreen composition according to claim 1 wherein the concentration of component (A)
is present in an amount from about 0.01 weight percent to about 10 weight percent based on the weight of the total composition.

16. The sunscreen composition according to claim 1 which is formulated as an Oil-in-Water (O/W), Water-in-Oil (W/O), Oil-in-Water-Oil (O/W/O), Water-Oil-in-Water (W/O/W), PIT emulsions or micro-emulsions.

17. The sunscreen composition according to claim 1 which is formulated as gels, lotions, milks, sprays, alcoholic or aqueous/alcoholic lotions, aerosol, wax/fat compositions, stick preparations, powders, tablets, foams or ointments.

18. A rinse-off or leave-on sunscreen composition incorporating the composition according to claim 1.

19. A method of improving the water-resistance of a sunscreen composition on skin or hair which method comprises
applying to said skin or hair the composition according to claim 1.

20. A method of improving sunscreen efficacy of a sunscreen composition on skin or hair by which method comprises
applying the composition according to claim 1 onto said hair or skin.

21. A substantially anhydrous sunscreen alcoholic formulations, which incorporates the composition according to claim 1.

22. The substantially anhydrous sunscreen alcoholic formulation according to claim 21 wherein the component A ranges from about 0.0001 to about 0.0500 weight percent of the substantially anyhydrous sunscreen alcoholic formulation.

23. The substantially anhydrous sunscreen alcoholic formulation according to claim 21, wherein the UV absorber is selected from the group b₃ which is at least one oil soluble organic UV absorber.
